(19) United States
(12) Patent Application Publication
(10) Pub. No.: US 2023/0147257 A1
(43) Pub. Date: May 11, 2023

(54) PYRIDOPYRIMIDINONE DERIVATIVES AND THEIR USE AS ARYL HYDROCARBON RECEPTOR MODULATORS

(71) Applicant: DONG-A ST CO., LTD., Seoul (KR)

(72) Inventors: Minsoo SONG, Daegu (KR); Ga Young PARK, Daegu (KR); Jihee KANG, Daegu (KR); Eunhye LEE, Daegu (KR); Yoojin PARK, Daegu (KR); Yujeong CHOI, Daegu (KR); Soong-Hyun KIM, Daegu (KR); Eun Bi KO, Daegu (KR); Seri BAE, Daegu (KR); Jung-Sang PARK, Osan-si (KR); Daewon CHA, Suwon-si (KR); Wonhyung LEE, Seoul (KR); Min Sung JOO, Seongnam-si (KR); Taeyoung YOON, Seongnam-si (KR); Hyounmie DOH, Yongin-si (KR); Hyun Jung SUNG, Suwon-si (KR); Bo Ryeong LEE, Seoul (KR); Yoonjung KIM, Suwon-si (KR)

(21) Appl. No.: 17/906,745

(22) PCT Filed: Apr. 19, 2021
(86) PCT No.: PCT/KR2021/004904

§ 371 Date: Sep. 19, 2022

Related U.S. Application Data
(60) Provisional application No. 63/011,351, filed on Apr. 17, 2020.

(51) Int. Cl. C07D 471/04; A61P 35/00

(52) U.S. Cl. CPC C07D 471/04; A61P 35/00

(57) ABSTRACT

The present invention relates to novel compounds effective as modulators Aryl hydrocarbon receptor (AhR), pharmaceutical composition comprising the compounds for the modulation of AhR, or prevention or treatment of a disease, disorder, or condition associated with AhR activity, as an active ingredient, and thus, can be useful as a medication for the prevention or treatment of a disease, disorder, or condition associated with AhR activity, in particular, cancer, cancerous condition, tumor, fibrotic disease, condition with dysregulated immune responses, etc.

### TECHNICAL FIELD

The present invention relates to novel pyridopyrimidinone derivatives that can modulate the activities of aryl hydrocarbon receptor (AhR). The compounds of formula (I) of the present invention can also be used for inhibiting the growth of cancer cells, tumor cell metastasis and invasion and for the treatment of diseases related with dysregulated immune responses associated with AhR signaling (a sole agent or in combination with other active ingredients).

### BACKGROUND ART

Aryl hydrocarbon receptor (AhR) is a ligand-activated transcription factor and is well-known as an important intracellular chemosensor responsive to both natural and man-made environmental compounds. As is well known, the AhR is a member of the periodic circadian protein (PER)-AhR nuclear translocator (ARNT)-single-minded protein (SIM) superfamily of transcription factors in which the PER-ARNT-SIM(PAS) domain senses ligands. (Burbach et al, PNAS Sep. 1, 1992 89 (17) 8185-8189) The AhR, activated by several binding ligands translocates to the nucleus and dimerizes with its partner protein, the ARNT. This heterodimeric complex interacts with the xenobiotic response elements (XREs) and it control the expression of AhR related genes directly or indirectly. One of the endogenous ligands to be well-characterized is kynurenine, generated by TDO (Opitz et al, Nature, Nature. 2011 Oct. 5; 478(7368):197-203) or IDO (Mezrich, J Immunol. 2010 Sep. 15; 185(6):3190-8.). Recent studies found that high concentrations of kynurenine in the plasma of diverse cancer patients and a high serum Kyn/Trp ratio correlates with poor prognosis after PD-1 blockade in several cancer types, including lung cancer, melanoma, and renal cell carcinomas. (Haoxin Li et al, Nat Commun. 2019 Sep. 25; 10(1):4346)

It has been well-known lately that AhR regulates the functions of a plethora of cells of both the innate and adaptive immune system. Activated AhR attenuates the induction of cytokines that promote the polarization of pathogenic T cell subsets and reduces MHC class II expression. In addition, AhR activation by agonist or modulator, inhibits the differentiation of helper Th17 cell and stabilizes regulatory T cell. Invigorated AhR also induces the generation of its ligands via a positive feedforward loop involving indolamine 2,3-dioxygenase 1 (IDO1). (Nguyen et al., PNAS, 2010, 107(46):19961-19966, Mascanfroni, I. D. et al. Nat. Med., 2015, 21:638-646) As an immune escape mechanism, Tumor-repopulating cells (TRCs) drive PD-1 upregulation in CD8+ T cells through a Kyn-AhR pathway. (Yuying Liu et al, Cancer cell, 2018).

Moreover, several studies have shown that AhR signaling plays important roles in diverse disease such as autoimmunity, infection, and cancer. AhR signaling may be related to autoimmune diseases, including rheumatoid arthritis (RA), systemic lupus erythematosus (SLE), multiple sclerosis (MS). (Xiao-Song Wang et al, Inflammopharmacology, 2020 February; 28(1):63-81) Constitutive AhR activation reduces the type I IFN (IFN-I) antiviral response (Yamada et al, Nat immunol, 2016 June; 17(6):687-94). The AhR activation is induced by multiple viruses to evade the host immune response, a strategy exploited in mouse models to limit the replication of Zika virus, SARS-COV-2 infection. (Federico Giovannoni et al, Cell Research, 2021 December, 31:1-2) The AhR may affect the proliferation, tissue invasion, metastasis, and angiogenesis of cancer cells (Jae Eun Cheong et al, Trends in Pharmacological Sciences, 2018 March; 39(3):307-325). In addition, many cancer types can escape from immune recognition via an AhR pathway. Developing AhR-targeted therapeutics could be the potential opportunities to overcome immune related diseases.

### DISCLOSURE

### Technical Problem

Therefore, it is an object of the present invention to provide novel compounds, or an enantiomer, diastereomer, racemate, solvate, hydrate or pharmaceutically acceptable salt thereof as modulators of AhR.

It is an object of the present invention to provide a pharmaceutical composition for the modulation of AhR activity, comprising the compounds as modulators of AhR

It is an object of the present invention to provide a pharmaceutical composition for the prevention or treatment of disease, disorder, or condition associated with AhR activity such as a cancer or an autoimmune disease, comprising the compounds as modulators of AhR.

It is an object of the present invention to provide a method for modulating AhR activity by administering the compounds as modulators of AhR.

It is an object of the present invention to provide a method for preventing or treating prostaglandin related diseases by administering the compounds as modulators of AhR.

It is an object of the present invention to provide a use of the prostaglandin analog for the modulation of AhR activity, or the prevention or treatment of disease, disorder, or condition associated with AhR.

### Technical Solution

### SUMMARY OF THE INVENTION

The present invention provides novel compounds, and pharmaceutical acceptable compositions are effective as modulators or inhibitors of AhR. The compounds are represented by formula (I)

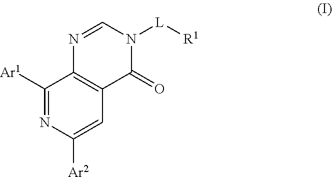

wherein:

Ar¹ and Ar² are independently selected from a group consisting of halo, substituted or unsubstituted mono- or bicyclic C₆₋₁₀ aryl, substituted or unsubstituted mono- or bicyclic C₅₋₁₀ heteroaryl and substituted or unsubstituted mono- or bicyclic C₃₋₁₀ heterocycloalkyl;

L is absent(direct bond), H, halo, cyano, hydroxy, amino, nitro, ether(-O—), thioether(-S—), sulfinyl(-SO—), sulfonyl(-SO₂—), sulfonylamido(-SO₂NR²—), aminosulfonyl(-NR²SO₂—), carbonyl(-(CO)—), amido(-(CO)NR²—), reverse amido(-NR²(CO)—), ester(-(CO)O—), substituted or unsubstituted C₁₋₅ alkyl, substituted or unsubstituted mono- or bicyclic C₃₋₁₀ cycloalkyl, substituted or unsubstituted mono- or bicyclic C₄₋₁₀ heterocycloalkyl, substituted or unsubstituted mono- or bicyclic C₆₋₁₀ aryl and substituted or unsubstituted mono- or bicyclic C₅₋₁₀ heteroaryl;

R¹ is absent(direct bond), H, halo, cyano, hydroxy, amino, NHR³, OR³, phosphate, substituted or unsubstituted C₁₋₃ alkyl phosphate, substituted or unsubstituted C₁₋₅ alkyl, sulfinic acid(-SO—H), sulfonic acid(-SO₂—H), sulfonylamide(-SO₂NR²₂), aminosulfonic acid(-NR²SO₂—H), carboxylic acid(-(CO)—H), carbonyl((-(CO)R²), amide(-(CO)NR²₂), reverse alkyl amide(—NH(CO)—R²), alkyl ester(-(CO)O—R²), sulfonate(-SO₂—R²), C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, substituted or unsubstituted mono- or bicyclic C₃₋₁₀ heterocycloalkyl and substituted or unsubstituted mono- or bicyclic C₅₋₁₀ heteroaryl;

R² is H, halo, hydroxy, amino, substituted or unsubstituted C₁₋₅ alkyl, substituted or unsubstituted C₁₋₅ alkoxy, substituted or unsubstituted C₃₋₈ cycloalkyl and substituted or unsubstituted C₁₋₅ alkyl carboxylic acid;

R³ is H, substituted or unsubstituted C₁₋₅ alkyl, C₁₋₅ alkylacetyl(alkyl-CO—), C₁₋₅ sulfonylalkyl(alkyl-SO₂—), C₁₋₅ sulfonylamidoalkyl(alkyl-SO₂NR²₂), C₁₋₅ amidoalkyl(alkyl-(CO)NR²₂), C₁₋₅ reverse amidoalkyl(alkyl-NR²(CO)—), substituted or unsubstituted C₁₋₅ alkoxy and substituted or unsubstituted C₁₋₅ alkyl carboxylic acid.

In some embodiments of these aspects and all such aspects described herein, the AhR modulator of Formula (I) is an AhR modulator or AhR antagonist.

In some aspects, described herein are methods of modulating AhR activity, more specifically constitutive AhR activity in a subject in need thereof. Such methods comprise administering to a subject having constitutive AhR activity a therapeutically effective amount of an AhR modulator, such as an AhR antagonist of Formula (I), described herein. In some embodiments of these aspects and all such aspects described herein, the methods further comprise the step of selecting the subject having constitutive AhR activity.

Compounds of formula (I) of the present invention demonstrate a valuable pharmacological spectrum of action, which could not have been predicted. Compounds of the present invention have surprisingly been found to effectively inhibit AhR and it is possible therefore that said compounds be used for the treatment or prophylaxis of a disease or condition mediated by aryl hydrocarbon receptor (AhR), preferably cancer, cancerous conditions, tumor, fibrotic disorders, or conditions with dysregulated immune responses or other disorders associated with aberrant AhR signaling, in humans and animals.

Examples of said diseases related with dysregulated immune response associated with AhR signaling are sepsis (SIRS), multiple organ failure (MODS, MOF), inflammatory disorders of the kidney, chronic intestinal inflammations (IBD, Crohn's disease, UC), pancreatitis, peritonitis, inflammatory skin disorders and inflammatory eye disorders, autoimmune diseases, such as rheumatoid diseases including rheumatoid arthritis (RA), systemic lupus erythematosus (SLE), multiple sclerosis (MS), etc.

Examples of said fibrotic disorders are fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

In other aspects, described herein are methods of treating a cancer or a cancerous condition by modulating AhR activity. Such methods comprise administering to a subject having a cancer or cancerous condition a therapeutically effective amount of any of the pharmaceutical compositions comprising an AhR modulator, such as an AhR antagonist of Formula (I), described herein.

In some aspects, described herein are methods of inhibiting tumor cell invasiveness in a subject having a cancer, a cancerous condition, or a tumor. Such methods comprise administering to a subject having a cancer or a tumor a therapeutically effective amount of any of the pharmaceutical compositions comprising an AhR modulator, such as an AhR antagonist of Formula (I), described herein.

In some embodiments of these aspects and all such aspects described herein, the methods further comprise the step of selecting the subject having a cancer, a cancerous condition, or a tumor.

Said cancer, cancerous condition, or tumor particularly suitable for treatment with an AHR inhibitor of the present invention are liquid and solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, triple negative breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, glioblastoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Examples of ovarian cancer include, but are not limited to serous tumour, endometrioid tumour, mucinous cystadenocarcinoma, granulosa cell tumour, Sertoli-Leydig cell tumour and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumours of the digestive tract include, but are not limited to, anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumours.

Tumours of the urinary tract include, but are not limited to, bladder, penile, kidney, renal pelvis, ureter, urethral and human papillary renal cancers.

Examples of kidney cancer include, but are not limited to renal cell carcinoma, urothelial cell carcinoma, juxtaglomerular cell tumour (reninoma), angiomyolipoma, renal oncocytoma, Bellini duct carcinoma, clear-cell sarcoma of the kidney, mesoblastic nephroma and Wilms' tumour.

Examples of bladder cancer include, but are not limited to transitional cell carcinoma, squamous cell carcinoma, adenocarcinoma, sarcoma and small cell carcinoma.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The term “treating” or “treatment” as stated throughout this document is used conventionally, for example the management or care of a subject for the purpose of combating, alleviating, reducing, relieving, improving the condition of a disease or disorder, such as a carcinoma.

The compounds or of the present invention can be used in particular in therapy and prevention, i.e. prophylaxis, of tumour growth and metastases, especially in solid tumours of all indications and stages with or without pre-treatment of the tumour growththe cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia. In some such embodiments, the cancer is a hepatocellular cancer.

Some embodiments of these methods can further comprise administration or treatment with one or more additional anti-cancer therapies. In some such embodiments, the additional anti-cancer therapy comprises surgery, radiation therapy, biotherapy, immunotherapy, chemotherapy, or any combination thereof.

Some embodiments of these methods can further comprise administration or treatment with one or more anti-cancer therapeutic agents. In some such embodiments, the anti-cancer therapeutic agent is a chemotherapeutic agent, a growth inhibitor agent, an anti-angiogenesis agent, a cytotoxic agent, an anti-hormonal agent, a prodrug, or a cytokine.

In a further embodiment of the present invention, the compounds of formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of formula (I) of the present invention prior to the treatment of the cell to cause or induce cell death. In one aspect, after the cell is treated with one or more compounds of formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e. after treating a cell with one or more compounds of formula (I) of the present invention to sensitize the cell to cell death, the cell is treated with at least one DNA damaging agent to kill the cell. DNA damaging agents useful in the present invention include, but are not limited to, chemotherapeutic agents (e.g. cisplatin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of formula (I) of the present invention is administered to a cell prior to the radiation or other induction of DNA damage in the cell. In another aspect of the invention, a compound of general formula (I) of the present invention is administered to a cell concomitantly with the radiation or other induction of DNA damage in the cell. In yet another aspect of the invention, a compound of formula (I) of the present invention is administered to a cell immediately after radiation or other induction of DNA damage in the cell has begun.

In another aspect, the cell is in vitro. In another embodiment, the cell is in vivo. The compounds of the present invention can be administered as the sole pharmaceutical agent or in combination with one or more other pharmaceutically active ingredients where the combination causes no unacceptable adverse effects.

The present invention also covers such pharmaceutical combinations. For example, the compounds of the present invention can be combined with: 131 1-chTNT, abarelix, abiraterone, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, aprepitant, arcitumomab, arglabin, arsenic trioxide, asparaginase, atezolizumab, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (1231), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumab, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, roniciclib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the invention can further be combined with other reagents targeting the immune system, such as immune checkpoint inhibitors, e.g. aPD-1/-L1 axis antagonists.

PD-1, along with its ligands PD-L1 and PD-L2, function as negative regulators of T cell activation. AHR suppresses immune cell function while increasing cancer cell proliferation and motility. PD-L1 is overexpressed in many cancers and overexpression of PD-1 often occurs concomitantly in tumor infiltrating T cells. Thus results in attenuation of T cell activation and evasion of immune surveillance, which contributes to impaired antitumor immune responses. (Keir M E et al. (2008) Annu. Rev. Immunol. 26:677).

Simultaneously targeting both the PD-1/-L1 axis and AHR enhances antitumor immune responses more than in an additive manner, leading to a reduction of tumor growth that is unexpected.

Thus, compositions comprising a PD-1/-L1 axis antagonist and an AHR antagonist are surprisingly effective in enhancing an immune response and in the treatment of cancer.

In addition, the inventive compounds can also be used as a therapeutic in a variety of other disorders wherein AHR is involved.

Examples of other disorders associated with aberrant AhR signaling inflammation are vaccination for infection & cancer, viral infections, obesity and diet-induced obesity, adiposity, metabolic disorders, hepatic steatosis and uterine fibroids (uterine leiomyoma or uterine myoma) in women, chronic renal disorders, acute and chronic renal insufficiency, diabetic, inflammatory or hypertensive nephropaties, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer, and Creutzfeld-Jakob.

Also provided herein, in other aspects, are pharmaceutical compositions comprising an AhR modulator, such as an AhR antagonist of Formula (I), and pharmaceutically acceptable excipients.

In some aspects, pharmaceutical compositions comprising an AhR modulator, such as an AhR antagonist of Formula (I), are provided for use in for modulating constitutive AhR activity in a subject in need thereof.

In some aspects, pharmaceutical compositions comprising an AhR modulator, such as an AhR antagonist of Formula (I), are provided for use in treating a cancer or a cancerous condition by modulating AhR activity.

In some aspects, pharmaceutical compositions comprising an AhR modulator, such as an AhR antagonist of Formula (I), are provided for use in inhibiting proliferation, tissue invasion, metastasis and angiogenesis of cancer cells in a subject having a cancer, a cancerous condition, or a tumor.

In some embodiments of these aspects and all such aspects described herein, the use further comprises the step of selecting the subject having a cancer, a cancerous condition, or a tumor. In some such embodiments, the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia. In some such embodiments, the cancer is a hepatocellular cancer.

In some embodiments of these aspects and all such aspects described herein, the use further comprises one or more additional anti-cancer therapies. In some such embodiments, the additional anti-cancer therapy comprises surgery, radiation therapy, biotherapy, immunotherapy, or chemotherapy.

In some embodiments of these aspects and all such aspects described herein, the use further comprises one or more anti-cancer therapeutic agents. In some such embodiments, the anti-cancer therapeutic agent is a chemotherapeutic agent, a growth inhibitor agent, an anti-angiogenesis agent, a cytotoxic agent, an anti-hormonal agent, a prodrug, or a cytokine.

### Advantageous Effects

The novel compounds of Formula (I) according to the present invention effectively modulate AhR activity, and therefore they are useful as a therapeutic or prophylactic drug for various disease, disorder, or condition associated with AhR activity such as cancer, cancerous condition, tumor, fibrotic disease, conditions with dysregulated immune responses including autoimmune disease such as rheumatoid arthiritis, systemic lupus erythematosus (SLE), multiple sclerosis (MS), or other disorders associated with aberrant AhR signaling etc.

### BEST MODE

Hereinafter, the present invention will be described in more detail.

Unless otherwise defined, all technical terms used herein have the same meaning as commonly understood by one of ordinary skill in the art to which this invention belongs. Also, although the invention has been described in conjunction with specific methods and samples, their analogs or equivalents should be within the scope of the present invention. Furthermore, the numerical values set forth herein are considered to include the meaning of “about” unless explicitly stated. All publications and other references mentioned herein are hereby incorporated by reference in their entirety.

The definition of residues used herein is described in detail. Unless otherwise indicated, each residue has the following definition and is used in the sense as commonly understood by one of ordinary skill in the art.

As used herein, the term “halo” “halogen”, “halide (s)” includes fluoro, chloro, bromo and iodo.

As used herein, the “alkyl” refers to an aliphatic hydrocarbon radical, and includes both linear and branched hydrocarbon radicals. For example, C₁₋₆ alkyl is an aliphatic hydrocarbon having 1 to 6 carbon atoms and includes methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl. Unless otherwise defined, the alkyl refers to C₁₋₆ alkyl, preferably C₁₋₄ alkyl, more preferably C₁₋₃ alkyl.

As used herein, the “alkenyl” refers to an aliphatic hydrocarbon radical comprising at least one carbon-carbon double bond, and includes both linear and branched hydrocarbon radicals. The unlimited example of the “alkenyl” is vinyl, allyl, but-1-enyl or but-2-enyl.

As used herein, the “alkynyl” refers to an aliphatic hydrocarbon radical comprising at least one carbon-carbon triple bond, and includes both linear and branched hydrocarbon radicals. The unlimited example of the “alkynyl” is ethynyl, propargyl, but-1-ynyl or but-2-ynyl.

As used herein, the “haloalkyl” refers to an alkyl group substituted with one or more halogen atom, and the alkyl group is defined as above. The “halo” refers to F, Cl, Br, or I, and the term is compatibly used with the term “halogen”. Unless otherwise defined, the haloalkyl refers tofluoromethyl, difluoromethyl, chloromethyl, trifluoromethyl or 2,2,2-trifluoromethyl.

As used herein, the term “alkoxy” refers to —O-alkyl or alkyl-O— group, and the alkyl group is defined as shown above. For example, it includes methoxy, ethoxy, n-propoxy, n-butoxy and t-butoxy.

As used herein, the “alkoxyalkyl” refers to alkyl-O-alkyl group, and the alkyl group is defined as above. The unlimited example is methoxymethyl, ethoxymethyl, methoxyethyl or isopropoxymethyl.

As used herein, the term “hydroxy” or “hydroxyl” alone or in combination with other terms means —OH.

As used herein, “cyano” refers to —CN, “cyanoalkyl” refers to alkyl substituted with —CN, wherein the alkyl group is as defined above.

As used herein, “amino” refers to —NH₂; and “nitro” refers to —NO₂.

As used herein, “carboxy” refers to —C(O)—OH group.

As used herein, “ester” refers to a group of —C(O)—OR, where R is alkyl may be C₁₋₁₀, preferably C₁₋₈, C₁₋₆ or C₁₋₄ alkyl. Such ester groups may or may not be substituted with one or more suitable substituents.

As used herein, the term “cycloalkyl” refers to a cyclic alkyl which may be substituted or unsubstituted, and for example, the C₃₋₂₀ cycloalkyl represents a monovalent saturated hydrocarbon ring system having 3 to 20 carbon atoms. Examples of the cycloalkyl include, but are not limited to, cyclopropyl, cyclobutyl, cyclopentyl, cyclohexyl, cycloheptyl, cyclooctyl and the like. Preferably, unless otherwise defined, the cycloalkyl may be C₃₋₈ cycloalkyl, or C₃₋₆ cycloalkyl.

As used herein, the term “aryl” refers to a monovalent aromatic hydrocarbon having, for example, 6 to 20 carbon atoms (C₆₋₂₀) that is derived by the removal of one hydrogen atom from a single carbon atom of a parent aromatic ring system. The aryl may include a bicyclic radical containing an aromatic ring fused to a saturated or partially unsaturated ring. Exemplary aryl groups may include radicals derived from benzene (phenyl), substituted phenyl, biphenyl, naphthyl, toluyl, naphthalenyl, anthracenyl, indenyl, indanyl, and the like. Unless otherwise defined, the aryl refers to C₆₋₁₂ aryl, preferably C₆₋₁₀ aryl.

As used herein, the “heteroaryl” refers to a monovalent or divalent substituent derived from a monoheterocyclic or polyheterocyclic aromatic hydrocarbon having 1 to 10 carbon ring members containing one or more, preferably one to three, heteroatoms selected among N, O, and S. Examples of the heteroaryl include, but are not limited to, thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl,1,2,4-oxadiazolyl,1,3,4-oxadiazolyl, 1,2,4-thiadiazolyl, 1,3,4-thiadiazolyl, triazolyl, tetrazolyl, triazinyl, indolyl, and the like. Examples of the bicyclic heteroaryl includeindolyl, benzothiophenyl, benzofuranyl, benzimidazolyl, benzoxazolyl, benzisoxazolyl, benzthiazolyl, benzthiadiazolyl, quinolinyl, isoquinolinyl, furinyl, furopyridinyl, octahydropyranopyridine, benzodioxolyl and similar groups thereof, but are not limited thereto. Unless otherwise defined, the heteroaryl is C₃₋₁₀ heteroaryl, preferably C₃₋₇ heteroaryl, more preferably C₃₋₅ heteroaryl.

As used herein, the “heterocycloalkyl” refers to monocyclic, bicyclic, tricyclic or higher cyclic alkyl having 3 to 10 carbon ring members containing one or more, for example, one to four, heteroatoms selected among N, O, and S. In addition, the heterocycle according to the present invention may also be a fused or bridged heterocycloalkyl. Examples of non-aromatic rings include azetidinyl, oxetanyl, tetrahydrothienyl, tetrahydrofuranyl, pyrrolinyl, pyrrolidinyl, imidazolinyl, imidazolidinyl, oxazolinyl, oxazolidinyl, oxapiperazinyl, oxapiperidinyl, pyrazolinyl, pyrazolidinyl, thiazolinyl, thiazolidinyl, tetrahydrofuranyl, tetrahydrofuryl, tetrahydroisothiazolyl, tetrahydrooxazolyl, tetrahydroisoxazolyl, piperidinyl, piperazinyl, tetrahydropyranyl, dihydropyranyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, indolinyl, indolinylmethyl, thiomorpholinyl, azepanyl, diazepanyl, N-oxide, azaadamantanyl, diazamantanyl, and the like, but are not limited thereto. Attachment of a heterocycloalkyl substituent can occur via a carbon atom or a heteroatom. A heterocycloalkyl group may be optionally substituted with one or more suitable groups via one or more aforementioned groups. Unless otherwise defined, heterocycloalkyl refers to heterocycloalkyl having 3 to 10 carbon ring members, preferably C₃₋₇ heterocycloalkyl, more preferably heterocycloalkyl having 3 to 5 carbon ring atoms.

Unless otherwise specified herein, the term “substituted” means that at least one hydrogen atom is substituted by one to three substituents selected from the group consisting of a halogen atom (e.g., F, Cl, Br, or I), a cyano group, a hydroxyl group, a thiol group, a nitro group, an amino group, an imino group, an azido group, an amidino group, a hydrazino group, a hydrazono group, an oxo group, a carbonyl group, a carbamyl group, an ester group, an ether group, a carboxyl group or a salt thereof, a sulfonic acid group or a salt thereof, phosphoric acid or a salt thereof, a C₁₋₆ alkyl group, a halo C₁₋₆ alkyl group, a C₂₋₆ alkenyl group, a halo C₂₋₆ alkenyl group, a C₂₋₆ alkynyl group, a halo C₂₋₆ alkynyl group, a C₁₋₆ alkoxy group, a halo C₁₋₆ alkoxy group, a C₁₋₂₀ alkylthio group, a C₃₋₂₀ carbocyclic group (e.g., a C₃₋₉ cycloalkyl group, a halo C₃₋₉ cycloalkyl group, a C₃₋₉ cycloalkenyl group, a halo C₃₋₉ cycloalkenyl group, a C₁₋₉ heterocycloalkyl group, a halo C₁₋₉ heterocycloalkyl group, a C₂₋₉ heterocycloalkenyl group, a halo C₂₋₉ heterocycloalkenyl group) and a C₁₋₂₀ heterocyclic group (e.g., a C₆₋₂₀ aryl group, a C₆₋₂₀ aryloxy group, a C₆₋₂₀ arylthio group, a C₂₋₂₀ heteroaryl group, a C₂₋₂₀ heteroaryloxy group, a C₂₋₂₀ heteroarylthio group).

Aryl Hydrocarbon Receptor

The Aryl Hydrocarbon Receptor (“AhR”) is a ligand-dependent member of the family of basic-helix-loop-helix transcription factors that has been found to be activated by numerous structurally diverse synthetic and naturally occurring compounds, such as polycyclic aromatic hydrocarbons, indoles, and flavonoids. In the absence of bound ligand, the AhR is present in a latent conformation in the cytoplasmic compartment of the cell associated with two molecules of the molecular chaperone heat shock protein 90 (“hsp90”), an immunophilin-like protein, XAP2, and the hsp90 interacting protein, p23.

The term “aryl hydrocarbon receptor” or “AhR” as used herein refers to the 848 amino acid polypeptide, as described by, e.g., NP_001612, together with any naturally occurring allelic, splice variants, and processed forms thereof. Typically, AhR refers to human AhR. The term AhR is also used to refer to truncated forms or fragments of the AhR polypeptide, comprising, for example, specific AhR domains. Reference to any such forms of the AhR can be identified in the application, e.g., by “AhR (122-224).”

AhR Modulators

The inventors of the present invention have discovered that the novel AhR modulator compounds described herein, such as the small molecules of Formula (I), modulate constitutive AhR activity, by functioning as AhR antagonists. Further, they have discovered that such AhR modulator compounds can inhibit cancer cell growth, as well as tumor invasion, metastasis and angiogenesis. Accordingly, described herein are novel modulators of the AhR and constitutive AhR signaling for use in therapeutic compositions for, and methods of, treating and inhibiting cancer growth and tumor cell invasion, and immune related diseases such as autoimmune diseases.

The AhR mediates a variety of functional responses, including, but not limited to de novo transcription of target genes or AhR battery genes having the DRE or XRE responsive element 5′-TNGCGTG-3′. Alternative pathways of AhR signaling have also been described, such as binding to retinoblastoma protein, estrogen receptor (ER), the transcription factor E2F1 and to the NFκB pathway subunits RelA and RelB. The AhR can also act as a ubiquitin ligase. Accordingly, signaling via the AhR comprises multiple pathways, including constitutive and non-constitutive AhR signaling pathways or signaling activity, as those terms are defined herein.

As used herein, “constitutive AhR signaling” refers to one or more signaling pathways mediated or regulated by the AhR that are activated or driven by one or more endogenous AhR ligands, or one or more environmental ligands, such as toxins or pollutants, that cause constitutive or long-term translocation of the AhR to the nucleus, and activation or modulation of one or more AhR battery genes involved in unregulated cell growth and proliferation, tumor cell invasiveness, or a combination thereof.

As used herein, “non-constitutive AhR signaling” refers to one or more signaling pathways mediated or induced by the AhR that does not cause constitutive or long-term translocation of the AhR to the nucleus, nor activation or modulation of one or more AhR battery genes involved in unregulated cell growth, tumor cell invasiveness, or a combination thereof. In some embodiments, non-constitutive AhR signaling does not cause upregulation of expression of CYP1A1, CYP1B1, or a combination thereof.

Accordingly, an “AhR modulator,” as the term is used herein refers to an agent, such as a compound of Formula (I), that modulates or causes or facilitates a qualitative or quantitative change, alteration, or modification in one or more processes, mechanisms, effects, responses, functions, activities or pathways mediated by the AhR receptor. Such changes mediated by an AhR modulator, such as an antagonist of the AhR described herein, can refer to a decrease in, inhibition of, or diversion of, constitutive activity of the AhR. The term “expression,” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “modulate” in reference to an Ahr modulator is used consistently with its use in the art, e.g., meaning to cause or facilitate a qualitative or quantitative change, alteration, or modification in one or more biological processes, mechanisms, effects, responses, functions, activities, pathways, or other phenomena of interest. Accordingly, as used herein, modulate refers to a qualitative or quantitative change, alteration, or modification in one or more processes, mechanisms, effects, responses, functions, activities or pathways mediated by the AhR receptor.

The term “agent” as used herein in reference to an AhR modulator means any compound or substance such as, but not limited to, a small molecule, nucleic acid, polypeptide, peptide, drug, ion, etc. An “agent” can be any chemical, entity, or moiety, including, without limitation, synthetic and naturally-occurring proteinaceous and non-proteinaceous entities. In some embodiments, an agent is a nucleic acid, a nucleic acid analogue, a protein, an antibody, a peptide, an aptamer, an oligomer of nucleic acids, an amino acid, or a carbohydrate, and includes, without limitation, proteins, oligonucleotides, ribozymes, DNAzymes, glycoproteins, siRNAs, lipoproteins, aptamers, and modifications and combinations thereof etc. In certain embodiments, as described herein, agents are small molecules having a chemical moiety. For example, chemical moieties include unsubstituted or substituted alkyl, aromatic, or heterocyclyl moieties. Compounds can be known to have a desired activity and/or property, e.g., modulate AhR activity, or can be selected from a library of diverse compounds, using, for example, the screening methods described herein.

In some embodiments, an AhR modulator selectively binds to the AhR. As used herein, “selectively binds” or “specifically binds” refers to the ability of an AhR antagonist, described herein to bind to a target, such as the AhR, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less. For example, if an antagonist described herein binds to the AhR with a K_{D} of 10⁻⁵ M or lower, but not to other molecules, or a related homologue, then the agent is said to specifically bind the AhR. Specific binding can be influenced by, for example, the affinity and avidity of the antagonist and the concentration of the antagonist used. The person of ordinary skill in the art can determine appropriate conditions under which the antagonists described herein selectively bind using any suitable methods, such as titration of an AhR antagonist in a suitable cell binding assay, such as those described herein.

In some aspects of the compositions and methods described herein, AhR modulators are AhR antagonists having the chemical structures of Formula (I), described herein.

As used herein, the AhR is an “AhR antagonist.” An AhR antagonist refers to an AhR inhibitor that does not provoke a biological response itself upon specifically binding to the AhR, but blocks or dampens agonist-mediated or ligand-mediated responses, i.e., an AhR antagonist can bind but does not activate the AhR, and the binding disrupts the interaction, displaces an AhR agonist, and/or inhibits the function of an AhR agonist. Thus, as used herein, an AhR antagonist does not function as an inducer of AhR activity when bound to the AhR, i.e., they function as pure AhR inhibitors. In some embodiments, an AhR antagonist selectively binds to the AhR.

In some embodiments of these aspects, the AhR antagonists described herein, such as the compounds of Formula (I) block constitutive AhR effector functions that mediate growth and progression of established tumors. In other embodiments, the small molecule AhR antagonists of Formula (I), described herein act as chemo preventatives by blocking AhR-mediated CYP1A1 induction and mutagen production on exposure to environmental ligands.

In some embodiments of these aspects, the AhR antagonists of Formula (I), described herein inhibit the early contributions of constitutively active AhR in driving malignant transformation. In some embodiments, the compounds of Formula (I) described herein inhibit constitutive AhR signaling-mediated cancer or tumor cell growth. In some embodiments, the compounds of Formula (I), described herein inhibit constitutive AhR signaling-mediated tumor invasion in driving malignant transformation.

Accordingly, provided for use in the various aspects described herein are AhR antagonist of Formula (I):

An aspect of the present invention relates to novel compounds that can modulate human aryl hydrocarbon receptor (AhR). These compounds bind specifically to AhR.

In some embodiments, the compound has the structure of formula (I), or an enantiomer, diastereomer, racemate, solvate, hydrate, or pharmaceutically acceptable salt thereof:

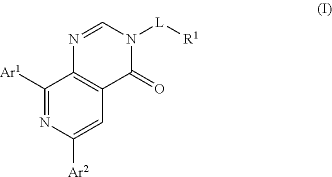

wherein:

Ar¹ and Ar² are independently selected from a group consisting of halo, substituted or unsubstituted mono- or bicyclic C₆₋₁₀ aryl, substituted or unsubstituted mono- or bicyclic C₅₋₁₀ heteroaryl and substituted or unsubstituted mono- or bicyclic C₃₋₁₀ heterocycloalkyl; L is absent(direct bond), H, halo, cyano, hydroxy, amino, nitro, ether(-O—), thioether(-S—), sulfinyl(-SO—), sulfonyl(-SO₂—), sulfonylamido(-SO₂NR²—), aminosulfonyl(-NR²SO₂—), carbonyl(-(CO)—), amido(-(CO)NR²—), reverse amido(-NR²(CO)—), ester(-(CO)O—), substituted or unsubstituted C₁₋₅ alkyl, substituted or unsubstituted mono- or bicyclic C₃₋₁₀ cycloalkyl, substituted or unsubstituted mono- or bicyclic C₄₋₁₀ heterocycloalkyl, substituted or unsubstituted mono- or bicyclic C₆₋₁₀ aryl and substituted or unsubstituted mono- or bicyclic C₅₋₁₀ heteroaryl;

R¹ is absent(direct bond), H, halo, cyano, hydroxy, amino, NHR³, OR³, phosphate, substituted or unsubstituted C₁₋₃ alkyl phosphate, substituted or unsubstituted C₁₋₅ alkyl, sulfinic acid(-SO—H), sulfonic acid(-SO₂—H), sulfonylamide(-SO₂NR²₂), aminosulfonic acid(-NR²SO₂—H), carboxylic acid(-(CO)—H), carbonyl((-(CO)R²), amide(-(CO)NR²₂), reverse alkyl amide(—NH(CO)—R²), alkyl ester(-(CO)O—R²), sulfonate(-SO₂—R²), C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, substituted or unsubstituted mono- or bicyclic C₃₋₁₀ heterocycloalkyl and substituted or unsubstituted mono- or bicyclic C₅₋₁₀ heteroaryl;

R² is H, halo, hydroxy, amino, substituted or unsubstituted C₁₋₅ alkyl, substituted or unsubstituted C₁₋₅ alkoxy, substituted or unsubstituted C₃₋₈ cycloalkyl and substituted or unsubstituted C₁₋₅ alkyl carboxylic acid;

R³ is H, substituted or unsubstituted C₁₋₅ alkyl, C₁₋₅ alkylacetyl(alkyl-CO—), C₁₋₅ sulfonylalkyl(alkyl-SO₂—), C₁₋₅ sulfonylamidoalkyl(alkyl-SO₂NR²₂), C₁₋₅ amidoalkyl(alkyl-(CO)NR²₂), C₁₋₅ reverse amidoalkyl(alkyl-NR²(CO)—), substituted or unsubstituted C₁₋₅ alkoxy and substituted or unsubstituted C₁₋₅ alkyl carboxylic acid.

In a preferred embodiment, the Ar¹ and the Ar² may be each independently halo, substituted or unsubstituted mono- or bicyclic C₆₋₁₀ aryl, substituted or unsubstituted monocyclic C₅₋₇ heteroaryl comprising one or more hetero atoms selected from the group consisting of N, O and S, or substituted or unsubstituted monocyclic C₅₋₇ heterocycloalkyl comprising one or more hetero atoms selected from the group consisting of N, O and S. More preferably, the Ar¹ and the Ar² may be each independently phenyl, monocyclic C₅₋₆ heteroaryl comprising one or two hetero atoms selected from the group consisting of N, O and S, or monocyclic C₅₋₆ heterocycloalkyl comprising one or two hetero atoms selected from the group consisting of N, O and S, which may be unsubstituted or substituted with halo, hydroxyl, amino, C₁₋₃ alkyl or C₁₋₃ alkoxy, where C₁₋₃ alkyl or C₁₋₃ alkoxy may be unsubstituted or substituted with one to three halo.

Far more preferably, the Ar¹ and the Ar² may be each independently phenyl, imidazole, pyridine, pyrimidine, piperidine or morpholine. Far more preferably, the Ar¹ and the Ar² may be unsubstituted or substituted with Cl, CH₃ or CF₃.

In a preferred embodiment, L is absent(direct bond), H, halo, cyano, hydroxy, amino, nitro, ether(-O—), thioether(-S—), sulfinyl(-SO—), sulfonyl(-SO₂—), sulfonylamido(-SO₂NR²—), aminosulfonyl(-NR²SO₂—), carbonyl(-(CO)—), amido(-(CO)NR²—), reverse amido(-NR²(CO)—), ester(-(CO)O—), substituted or unsubstituted mono- or bicyclic C₃₋₈ cycloalkyl, substituted or unsubstituted mono- or bicyclic C₃₋₈ heterocycloalkyl, substituted or unsubstituted mono- or bicyclic C₆₋₁₀ aryl and substituted or unsubstituted mono- or bicyclic C₅₋₈ heteroaryl, wherein the mono- or bicyclic C₃₋₈ heterocycloalkyl and mono- or bicyclic C₅₋₈ heteroaryl comprises one or more, preferably one or two heteroatoms selected from the group consisting of N, O and S.

More preferably, L is absent(direct bond), H, substituted or unsubstituted C₁₋₅ alkyl, 1,1-dioxydotetrahydrothiopyrane, piperidine, substituted or unsubstituted mono- or bicyclic C₃₋₆ cycloalkyl, where C₁₋₅ alkyl, substituted or unsubstituted mono- or bicyclic C₃₋₆ cycloalkyl may be substituted with one or more (preferably one to three) substituents selected from a group consisting of hydroxyl, halo, haloC₁₋₃ alkyl and C₁₋₃ alkyl.

In a preferred embodiment, R is absent, H, halo, cyano, hydroxy, amino, N(R³)₂, OR³, substituted or unsubstituted C₁₋₄ alkyl, carbonyl((-(CO)R²), C₃₋₈ cycloalkyl, C₁₋₄ alkylhydroxy, C₁₋₄ alkenylhydroxy, C₁₋₄ alkynylhydroxy, C₁₋₄ alkylamine, C₁₋₄ alkenylamine, C₁₋₄ alkynylamine, substituted or unsubstituted mono- or bicyclic C₃₋₈ heterocycloalkyl and substituted or unsubstituted mono- or bicyclic C₅₋₈ heteroaryl, wherein the mono- or bicyclic C₃₋₈ heterocycloalkyl and mono- or bicyclic C₅₋₈ heteroaryl comprises one or more, preferably one or two heteroatoms selected from the group consisting of N, O and S. More preferably, R¹ is absent, H, hydroxyl, —NH₂, —NH—C(O)CH₃, —NH—SO₂—CH₃, —C(O)OH, —SO2-CH3, —OC(O)—CH₃, —O—P(═O)(OCH₂CH₃)₂, —C(O)CH₃, or hydroxyl.

Further, in a more specific embodiment, the compound of the Formula I may be one selected from the group consisting of Compounds 1 to 96, as shown below:
1. 3-(3-hydroxycyclohexyl)-8-(1-methyl-1H-pyrazol-4-yl)-6-(4-(trifluoromethyl)phenyl)pyrido[3,4-d]pyrimidin-4(3H)-one;
2. 3-(3-hydroxycyclohexyl)-6,8-bis(1-methyl-1H-pyrazol-4-yl)pyrido[3,4-d]pyrimidin-4(3H)-one;
3. 3-(1-hydroxypropan-2-yl)-6,8-bis(1-methyl-1H-pyrazol-4-yl)pyrido[3,4-d]pyrimidin-4(3H)-one;
4. 3-(1-hydroxypropan-2-yl)-6-(1-methyl-1H-pyrazol-4-yl)-8-(4-(trifluoromethyl)phenyl)pyrido[3,4-d]pyrimidin-4(3H)-one;
5. 8-(4-chlorophenyl)-3-(1-hydroxypropan-2-yl)-6-(pyridin-3-yl)pyrido[3,4-d]pyrimidin-4(3H)-one;
6. 3-(1-hydroxypropan-2-yl)-6,8-bis(4-(trifluoromethyl)phenyl)pyrido[3,4-d]pyrimidin-4(3H)-one;
7. 2-(6-chloro-8-(4-chlorophenyl)-4-oxopyrido[3,4-d]pyrimidin-3(4H)-yl)propyl acetate;
8. 3-((1r,4r)-4-hydroxycyclohexyl)-8-(1-methyl-1H-pyrazol-4-yl)-6-(4-(trifluoromethyl)phenyl)pyrido[3,4-d]pyrimidin-4(3H)-one;
9. 3-((1r,4r)-4-hydroxycyclohexyl)-6,8-bis(1-methyl-1H-pyrazol-4-yl)pyrido[3,4-d]pyrimidin-4(3H)-one;
10. 6-(4-chlorophenyl)-3-((1r,4r)-4-hydroxycyclohexyl)-8-(1-methyl-1H-pyrazol-4-yl)pyrido[3,4-d]pyrimidin-4(3H)-one;
11. 3-(2-hydroxypropyl)-6,8-bis(1-methyl-1H-pyrazol-4-yl)pyrido[3,4-d]pyrimidin-4(3H)-one;
12. 3-(2-hydroxypropyl)-8-(1-methyl-1H-pyrazol-4-yl)-6-(4-(trifluoromethyl)phenyl)pyrido[3,4-d]pyrimidin-4(3H)-one;
13. 6-(4-chlorophenyl)-3-(2-hydroxypropyl)-8-(1-methyl-1H-pyrazol-4-yl)pyrido[3,4-d]pyrimidin-4(3H)-one;
14. 3-(2-hydroxypropyl)-8-(1-methyl-1H-pyrazol-4-yl)-6-(6-(trifluoromethyl)pyridin-3-yl)pyrido[3,4-d]pyrimidin-4(3H)-one;
15. 3-((1S,2R)-2-hydroxycyclohexyl)-8-(pyridin-3-yl)-6-(4-(trifluoromethyl)phenyl)pyrido[3,4-d]pyrimidin-4(3H)-one;
16. 3-((1R,2S)-2-hydroxycyclohexyl)-8-(pyridin-3-yl)-6-(4-(trifluoromethyl)phenyl)pyrido[3,4-d]pyrimidin-4(3H)-one;
17. 3-((1S,2R)-2-hydroxycyclohexyl)-8-(1-methyl-1H-pyrazol-4-yl)-6-(4-(trifluoromethyl)phenyl)pyrido[3,4-d]pyrimidin-4(3H)-one;
18. 3-((1R,2S)-2-hydroxycyclohexyl)-8-(1-methyl-1H-pyrazol-4-yl)-6-(4-(trifluoromethyl)phenyl)pyrido[3,4-d]pyrimidin-4(3H)-one;
19. 3-((1R,2S)-2-hydroxycyclohexyl)-8-(1-methyl-1H-pyrazol-4-yl)-6-(4-(trifluoromethyl)phenyl)pyrido[3,4-d]pyrimidin-4(3H)-one;
20. 6-(4-chlorophenyl)-3-((1S,2R)-2-hydroxycyclohexyl)-8-(1-methyl-1H-pyrazol-4-yl)pyrido[3,4-d]pyrimidin-4(3H)-one;
21. 6-(4-chlorophenyl)-3-((1S,2R)-2-hydroxycyclohexyl)-8-(pyridin-3-yl)pyrido[3,4-d]pyrimidin-4(3H)-one;
22. 8-(1-methyl-1H-pyrazol-4-yl)-3-(3,3,3-trifluoro-2-hydroxypropyl)-6-(4-(trifluoromethyl)phenyl)pyrido[3,4-d]pyrimidin-4(3H)-one;
23. 6-(4-chlorophenyl)-8-(1-methyl-1H-pyrazol-4-yl)-3-(3,3,3-trifluoro-2-hydroxypropyl)pyrido[3,4-d]pyrimidin-4(3H)-one;
24. 6-(4-chlorophenyl)-8-(pyridin-3-yl)-3-(3,3,3-trifluoro-2-hydroxypropyl)pyrido[3,4-d]pyrimidin-4(3H)-one;
25. 8-(pyridin-3-yl)-3-(3,3,3-trifluoro-2-hydroxypropyl)-6-(4-(trifluoromethyl)phenyl)pyrido[3,4-d]pyrimidin-4(3H)-one;
26. 6-(4-chlorophenyl)-3-(3-hydroxyphenyl)-8-(1-methyl-1H-pyrazol-4-yl)pyrido[3,4-d]pyrimidin-4(3H)-one;
27. 3-(3-hydroxyphenyl)-8-(1-methyl-1H-pyrazol-4-yl)-6-(4-(trifluoromethyl)phenyl)pyrido[3,4-d]pyrimidin-4(3H)-one;
28. 6-(4-chlorophenyl)-3-((1R,3S)-3-hydroxycyclopentyl)-8-(pyridin-3-yl)pyrido[3,4-d]pyrimidin-4(3H)-one;
29. 3-((1R,3S)-3-hydroxycyclopentyl)-8-(pyridin-3-yl)-6-(4-(trifluoromethyl)phenyl)pyrido[3,4-d]pyrimidin-4(3H)-one;
30. 6-(4-chlorophenyl)-3-((1R,3S)-3-hydroxycyclopentyl)-8-(1-methyl-1H-pyrazol-4-yl)pyrido[3,4-d]pyrimidin-4(3H)-one;
31. 3-((1R,3S)-3-hydroxycyclopentyl)-8-(1-methyl-1H-pyrazol-4-yl)-6-(4-(trifluoromethyl)phenyl)pyrido[3,4-d]pyrimidin-4(3H)-one;
32. 6-(4-chlorophenyl)-3-((1S,3R)-3-hydroxycyclopentyl)-8-(pyridin-3-yl)pyrido[3,4-d]pyrimidin-4(3H)-one;
33. 3-((1S,3R)-3-hydroxycyclopentyl)-8-(pyridin-3-yl)-6-(4-(trifluoromethyl)phenyl)pyrido[3,4-d]pyrimidin-4(3H)-one;
34. 6-(4-chlorophenyl)-3-((1S,3R)-3-hydroxycyclopentyl)-8-(1-methyl-1H-pyrazol-4-yl)pyrido[3,4-d]pyrimidin-4(3H)-one;
35. 3-((1S,3R)-3-hydroxycyclopentyl)-8-(1-methyl-1H-pyrazol-4-yl)-6-(4-(trifluoromethyl)phenyl)pyrido[3,4-d]pyrimidin-4(3H)-one;
36. 1-(6-(4-chlorophenyl)-4-oxo-8-(pyridin-3-yl)pyrido[3,4-d]pyrimidin-3(4H)-yl)-2-methylpropan-2-yl acetate;
37. 2-methyl-1-(4-oxo-8-(pyridin-3-yl)-6-(4-(trifluoromethyl)phenyl)pyrido[3,4-d]pyrimidin-3(4H)-yl)propan-2-yl acetate;
38. 6-(4-chlorophenyl)-3-(2-hydroxy-2-methylpropyl)-8-(pyridin-3-yl)pyrido[3,4-d]pyrimidin-4(3H)-one;
39. 3-(2-hydroxy-2-methylpropyl)-8-(pyridin-3-yl)-6-(4-(trifluoromethyl)phenyl)pyrido[3,4-d]pyrimidin-4(3H)-one;
40. 3-(2-hydroxy-2-methylpropyl)-8-(pyridin-3-yl)-6-(6-(trifluoromethyl)pyridin-3-yl)pyrido[3,4-d]pyrimidin-4(3H)-one;
41. 6-(4-chlorophenyl)-3-(1-hydroxy-3-methylbutan-2-yl)-8-(pyridin-3-yl)pyrido[3,4-d]pyrimidin-4(3H)-one;
42. 3-(1-hydroxy-3-methylbutan-2-yl)-8-(pyridin-3-yl)-6-(4-(trifluoromethyl)phenyl)pyrido[3,4-d]pyrimidin-4(3H)-one;
43. 3-(1-hydroxy-3-methylbutan-2-yl)-8-(pyridin-3-yl)-6-(6-(trifluoromethyl)pyridin-3-yl)pyrido[3,4-d]pyrimidin-4(3H)-one;
44. (S)-2-((6-(4-chlorophenyl)-2-(pyridin-3-yl)pyrimidin-4-yl)amino)propan-1-ol;
44. 3-(1-hydroxypropan-2-yl)-8-(pyridin-3-yl)-6-(4-(trifluoromethyl)phenyl)pyrido[3,4-d]pyrimidin-4(3H)-one;
45. 3-(1-hydroxypropan-2-yl)-8-(1-methyl-1H-pyrazol-4-yl)-6-(4-(trifluoromethyl)phenyl)pyrido[3,4-d]pyrimidin-4(3H)-one;
46. 6-(4-chlorophenyl)-3-(1-hydroxypropan-2-yl)-8-(pyridin-3-yl)pyrido[3,4-d]pyrimidin-4(3H)-one;
47. 2-(6-(4-chlorophenyl)-4-oxo-8-(pyridin-3-yl)pyrido[3,4-d]pyrimidin-3(4H)-yl)propyl diethyl phosphate;
48. 6-(4-chlorophenyl)-3-(1-hydroxypropan-2-yl)-8-(1-methyl-1H-pyrazol-4-yl)pyrido[3,4-d]pyrimidin-4(3H)-one;
49. 3-(1-hydroxypropan-2-yl)-8-(pyridin-3-yl)-6-(4-(trifluoromethoxy)phenyl)pyrido[3,4-d]pyrimidin-4(3H)-one;
50. 3-(1-hydroxypropan-2-yl)-8-(pyridin-3-yl)-6-(6-(trifluoromethyl)pyridin-3-yl)pyrido[3,4-d]pyrimidin-4(3H)-one;
51. 6-(4-chlorophenyl)-3-(1-hydroxybutan-2-yl)-8-(pyridin-3-yl)pyrido[3,4-d]pyrimidin-4(3H)-one;
52. 6-(4-chlorophenyl)-3-(1-hydroxybutan-2-yl)-8-(1-methyl-1H-pyrazol-4-yl)pyrido[3,4-d]pyrimidin-4(3H)-one;
53. 3-(1-hydroxybutan-2-yl)-8-(pyridin-3-yl)-6-(4-(trifluoromethyl)phenyl)pyrido[3,4-d]pyrimidin-4(3H)-one;
54. 3-(1-hydroxybutan-2-yl)-8-(1-methyl-1H-pyrazol-4-yl)-6-(4-(trifluoromethyl)phenyl)pyrido[3,4-d]pyrimidin-4(3H)-one;
55. 6-(4-chlorophenyl)-8-(3-fluorophenyl)-3-(1-hydroxybutan-2-yl)pyrido[3,4-d]pyrimidin-4(3H)-one;
56. 6-(4-chlorophenyl)-3-((1r,4r)-4-hydroxycyclohexyl)-8-(pyridin-3-yl)pyrido[3,4-d]pyrimidin-4(3H)-one;
57. 3-((1r,4r)-4-hydroxycyclohexyl)-8-(pyridin-3-yl)-6-(4-(trifluoromethyl)phenyl)pyrido[3,4-d]pyrimidin-4(3H)-one;
58. 6-(4-chlorophenyl)-3-((1s,4s)-4-hydroxycyclohexyl)-8-(pyridin-3-yl)pyrido[3,4-d]pyrimidin-4(3H)-one;
59. 3-(1-hydroxypropan-2-yl)-8-(1-methyl-1H-pyrazol-4-yl)-6-(4-(trifluoromethyl)phenyl)-2,3-dihydropyrido[3,4-d]pyrimidin-4(1H)-one;
60. 6-(4-chlorophenyl)-3-(2,3-dihydroxypropyl)-8-(pyridin-3-yl)pyrido[3,4-d]pyrimidin-4(3H)-one;
61. 6-(4-chlorophenyl)-3-(3-hydroxyphenyl)-8-(pyridin-3-yl)pyrido[3,4-d]pyrimidin-4(3H)-one;
62. 3-(3-hydroxyphenyl)-8-(pyridin-3-yl)-6-(4-(trifluoromethyl)phenyl)pyrido[3,4-d]pyrimidin-4(3H)-one;
63. 6-(4-chlorophenyl)-3-(3-hydroxycyclohexyl)-8-(pyridin-3-yl)pyrido[3,4-d]pyrimidin-4(3H)-one;
64. 6-(4-chlorophenyl)-3-(3-hydroxycyclohexyl)-8-(1-methyl-1H-pyrazol-4-yl)pyrido[3,4-d]pyrimidin-4(3H)-one;
65. 3-((1R,3S)-3-hydroxycyclopentyl)-8-(pyridin-3-yl)-6-(6-(trifluoromethyl)pyridin-3-yl)pyrido[3,4-d]pyrimidin-4(3H)-one;
65. 3-((1R,3S)-3-hydroxycyclopentyl)-8-(pyridin-3-yl)-6-(6-(trifluoromethyl)pyridin-3-yl)pyrido[3,4-d]pyrimidin-4(3H)-one;
66. 3-(2,3-dihydroxypropyl)-8-(pyridin-3-yl)-6-(4-(trifluoromethyl)phenyl)pyrido[3,4-d]pyrimidin-4(3H)-one;
67. 6-(4-chlorophenyl)-3-(2,3-dihydroxypropyl)-8-(pyridin-3-yl)pyrido[3,4-d]pyrimidin-4(3H)-one;
68. 3-(2,3-dihydroxypropyl)-6-(4-(4-methylpiperazin-1-yl)phenyl)-8-(pyridin-3-yl)pyrido[3,4-d]pyrimidin-4(3H)-one
69. 3-(1,3-dihydroxypropan-2-yl)-8-(pyridin-3-yl)-6-(4-(trifluoromethyl)phenyl)pyrido[3,4-d]pyrimidin-4(3H)-one;
70. 6-(4-chlorophenyl)-3-(1,3-dihydroxypropan-2-yl)-8-(pyridin-3-yl)pyrido[3,4-d]pyrimidin-4(3H)-one;
71. 6-(6-chloropyridin-3-yl)-3-((1R,3S)-3-hydroxycyclopentyl)-8-(pyridin-3-yl)pyrido[3,4-d]pyrimidin-4(3H)-one
72. 3-((1R,3S)-3-hydroxycyclopentyl)-8-(pyridin-3-yl)-6-(2-(trifluoromethyl)pyrimidin-5-yl)pyrido[3,4-d]pyrimidin-4(3H)-one, TFA salt;
73. 3-((1R,3S)-3-hydroxycyclopentyl)-6-(4-morpholinophenyl)-8-(pyridin-3-yl)pyrido[3,4-d]pyrimidin-4(3H)-one;
74. 6-(4′-chloro-[1,1′-biphenyl]-4-yl)-3-(1-hydroxypropan-2-yl)-8-(pyridin-3-yl)pyrido[3,4-d]pyrimidin-4(3H)-one;
75. 3-(1-hydroxypropan-2-yl)-6-(4-morpholinophenyl)-8-(pyridin-3-yl)pyrido[3,4-d]pyrimidin-4(3H)-one;
76. 3-(2-(methylsulfonyl)ethyl)-8-(pyridin-3-yl)-6-(4-(trifluoromethyl)phenyl)pyrido[3,4-d]pyrimidin-4(3H)-one;
76. 3-(2-(methylsulfonyl)ethyl)-8-(pyridin-3-yl)-6-(4-(trifluoromethyl)phenyl)pyrido[3,4-d]pyrimidin-4(3H)-one;
77. 6-(4-chlorophenyl)-3-(2-(methylsulfonyl)ethyl)-8-(pyridin-3-yl)pyrido[3,4-d]pyrimidin-4(3H)-one;
78. 3-(1,1-dioxidotetrahydro-2H-thiopyran-4-yl)-6-(4-morpholinophenyl)-8-(pyridin-3-yl)pyrido[3,4-d]pyrimidin-4(3H)-one;
79. 3-(2-(methylsulfonyl)ethyl)-6-(4-morpholinophenyl)-8-(pyridin-3-yl)pyrido[3,4-d]pyrimidin-4(3H)-one;
80. 3-(1,3-dihydroxypropan-2-yl)-6-(4-morpholinophenyl)-8-(pyridin-3-yl)pyrido[3,4-d]pyrimidin-4(3H)-one;
80. 3-(1,3-dihydroxypropan-2-yl)-6-(4-morpholinophenyl)-8-(pyridin-3-yl)pyrido[3,4-d]pyrimidin-4(3H)-one;
81. (R)-3-(2,3-dihydroxypropyl)-8-(pyridin-3-yl)-6-(4-(trifluoromethyl)phenyl)pyrido[3,4-d]pyrimidin-4(3H)-one;
82. 3-(2,3-dihydroxypropyl)-6-(4-morpholinophenyl)-8-(pyridin-3-yl)pyrido[3,4-d]pyrimidin-4(3H)-one;
83. 2-(6-(4-chlorophenyl)-4-oxo-8-(pyridin-3-yl)pyrido[3,4-d]pyrimidin-3(4H)-yl)propanoic acid, 2,2,2-trifluoroacetic acid salt;
84. 2-(4-oxo-8-(pyridin-3-yl)-6-(4-(trifluoromethyl)phenyl)pyrido[3,4-d]pyrimidin-3(4H)-yl)propanoic acid, 2,2,2-trifluoroacetic acid salt;
86. N-(2-(4-oxo-8-(pyridin-3-yl)-6-(4-(trifluoromethyl)phenyl)pyrido[3,4-d]pyrimidin-3(4H)-yl)propyl)acetamide;
85. 3-(1-aminopropan-2-yl)-8-(pyridin-3-yl)-6-(4-(trifluoromethyl)phenyl)pyrido[3,4-d]pyrimidin-4(3H)-one;
86. N-(2-(4-oxo-8-(pyridin-3-yl)-6-(4-(trifluoromethyl)phenyl)pyrido[3,4-d]pyrimidin-3(4H)-yl)propyl)acetamide;
87. 3-(1-aminopropan-2-yl)-6-(4-chlorophenyl)-8-(pyridin-3-yl)pyrido[3,4-d]pyrimidin-4(3H)-one;
88. N-(2-(6-(4-chlorophenyl)-4-oxo-8-(pyridin-3-yl)pyrido[3,4-d]pyrimidin-3(4H)-yl)propyl)acetamide;
89. N-(2-(6-(4-chlorophenyl)-4-oxo-8-(pyridin-3-yl)pyrido[3,4-d]pyrimidin-3(4H)-yl)propyl)methanesulfonamide;
90. 3-(1-aminopropan-2-yl)-6-(4-morpholinophenyl)-8-(pyridin-3-yl)pyrido[3,4-d]pyrimidin-4(3H)-one;
91. N-(2-(6-(4-morpholinophenyl)-4-oxo-8-(pyridin-3-yl)pyrido[3,4-d]pyrimidin-3(4H)-yl)propyl)methanesulfonamide;
92. N-(2-(6-(4-morpholinophenyl)-4-oxo-8-(pyridin-3-yl)pyrido[3,4-d]pyrimidin-3(4H)-yl)propyl)acetamide;
93. 3-(piperidin-4-yl)-8-(pyridin-3-yl)-6-(4-(trifluoromethyl)phenyl)pyrido[3,4-d]pyrimidin-4(3H)-one;
94. 6-(4-chlorophenyl)-3-(1-(methylsulfonyl)piperidin-4-yl)-8-(pyridin-3-yl)pyrido[3,4-d]pyrimidin-4(3H)-one;
95. 6-(4-chlorophenyl)-3-(1-(cyclopropylsulfonyl)piperidin-4-yl)-8-(pyridin-3-yl)pyrido[3,4-d]pyrimidin-4(3H)-one; and
96. 3-(1-acetylpiperidin-4-yl)-6-(4-chlorophenyl)-8-(pyridin-3-yl)pyrido[3,4-d]pyrimidin-4(3H)-one.

The compounds of the present invention may be synthesized by methods known in the art or by methods illustrated in Examples 1-96 below.

Pharmaceutical Compositions, Methods and Use

In a specific embodiment, the pharmaceutical composition and the method provided herein comprises the compound of Formula (I) or an enantiomer, diastereomer, racemate, solvate, hydrate, or pharmaceutically acceptable salt thereof.

The subject may be a mammal including human or a mammalian cell; for example, a mammal (e.g., human) suffering from the disease, disorder, or condition associated with AhR activity as described above or a mammalian cell isolated therefrom.

The compound as an active ingredient or the pharmaceutical composition may be administered orally or parenterally. For example, the parenteral administration may be performed by any one of intravenous injection, subcutaneous injection, intramuscular injection, intraperitoneal injection, endothelial administration, topical administration, intranasal administration, intrapulmonary administration, intrarectal administration, and the like.

The effective amount may refer to pharmaceutically and/or therapeutically effective amount, and may be prescribed depending on factors such as a type of preparation (formulation), administration route, the patient's age, body weight, gender, and/or pathologic conditions, and the like.

A pharmaceutically acceptable salt of the compound of Formula (I) may include addition salts formed by inorganic acids such as hydrochloride, sulfate, phosphate, hydrobromide, hydroiodide, nitrate, pyrosulfate, or metaphosphate, addition salts formed by organic acids such as citrate, oxalate, benzoate, acetate, trifluoroacetate, propionate, succinate, fumarate, lactate, maleate, tartrate, glutarate, or sulfonate, or metal salts such as lithium salt, sodium salt, potassium salt, magnesium salt and calcium salt, but is not limited thereto.

The pharmaceutical composition according to the present invention can be formulated into a suitable form together with a commonly used pharmaceutically acceptable carrier. The “pharmaceutically acceptable” refers to being physiologically acceptable, and not usually causing an allergic reaction or a similar reaction such as gastrointestinal disorders and dizziness when administered to humans. Further, the pharmaceutical composition of the present invention may be used after being formulated into an oral preparation, such as powders, granules, tablets, capsules, suspensions, emulsions, syrups, and aerosols, etc., and a parental preparation, such as epidermal formulations, suppositories, or sterile injection solutions, in accordance with a conventional method.

Examples of carriers, excipients and diluents that can be included in the composition, may include lactose, dextrose, sucrose, sorbitol, mannitol, xylitol, erythritol, maltitol, starch, arabic gum, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate, and mineral oil, but are not limited thereto. When formulated into a preparation, a diluting agent or an excipient, such as commonly-used fillers, stabilizing agents, binding agents, disintegrating agents, and surfactants can be used. Solid preparations for oral administration include tablets, pills, powders, granules, capsules, and the like, and these solid preparations may be prepared by mixing the compound of the present invention with at least one excipient, for example, starch, microcrystalline cellulose, sucrose, lactose, low-substituted hydroxypropyl cellulose, hypromellose or the like. In addition to the simple excipient, a lubricant such as magnesium stearate and talc are also used. Liquid preparations for oral administration include a suspension, a liquid for internal use, an emulsion, a syrup, etc. In addition to a commonly used simple diluent such as water and liquid paraffin, various excipients such as a humectant, a sweetener, an aromatic, a preservative, etc. may also be contained. Formulations for parenteral administration include a sterilized aqueous solution, a non-aqueous solution, a suspension, an emulsion, a lyophilized formulation and a suppository. The non-aqueous solution or suspension may contain propylene glycol, polyethylene glycol, a vegetable oil such as olive oil, an injectable ester such as ethyl oleate, etc. As a base of the suppository, witepsol, macrogol, tween 61, cocoa butter, laurin butter, glycerogelatin, etc. may be used. In order to formulate the formulation for parenteral administration, the compound of Formula I or a pharmaceutically acceptable salt thereof may be mixed in water together with sterilized and/or contain adjuvants such as preservatives, stabilizers, auxiliary agents such as wettable powder or emulsifying accelerators, salt for controlling osmotic pressure and/or buffers and the like, and other therapeutically useful substances, to prepare a solution or suspension, which is then manufactured in the form of an ampoule or vial unit administration.

The pharmaceutical composition including the compound of Formula I disclosed herein as an active ingredient may be administered to mammals such as mice, livestock, and humans by various routes for the modulation of AhR activity, or the prevention or treatment of a disease, disorder, or condition associated with AhR activity.

In some embodiment, the disease, disorder, or condition associated with AhR activity. may be a cancer, cancerous condition, tumor, fibrotic disorders, immune related disease or other disease related with AhR signaling.

In some embodiment, the diseases related with dysregulated immune response associated with AhR signaling are selected from the group consisting of sepsis (SIRS), multiple organ failure (MODS, MOF), inflammatory disorders of the kidney, chronic intestinal inflammations (IBD, Crohn's disease, UC), pancreatitis, peritonitis, inflammatory skin disorders and inflammatory eye disorders, autoimmune diseases, such as rheumatoid diseases including rheumatoid arthritis (RA), systemic lupus erythematosus (SLE), and multiple sclerosis (MS).

In some embodiment, the fibrotic disorders are selected from the group consisting of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

In some embodiments of the cancer, cancerous condition, or tumor particularly suitable for treatment with an AHR antagonist of the present invention are liquid and solid tumours, such as a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia.

In some embodiments, the pharmaceutical composition of the preset invention can be used together with one or more additional anti-cancer therapies. In some such embodiments, the additional anti-cancer therapy comprises surgery, radiation therapy, biotherapy, immunotherapy, chemotherapy, or any combination thereof.

In some embodiments, the pharmaceutical composition of the preset invention can be used together with anti-cancer therapeutic agents. In some such embodiments, the anti-cancer therapeutic agent is a chemotherapeutic agent, a growth inhibitor agent, an anti-angiogenesis agent, a cytotoxic agent, an anti-hormonal agent, a prodrug, or a cytokine.

Examples of other disorders associated with aberrant AhR signaling inflammation are vaccination for infection & cancer, viral infections, obesity and diet-induced obesity, adiposity, metabolic disorders, hepatic steatosis and uterine fibroids (uterine leiomyoma or uterine myoma) in women, chronic renal disorders, acute and chronic renal insufficiency, diabetic, inflammatory or hypertensive nephropaties, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer, and Creutzfeld-Jakob.

Also provided herein, in other aspects, are pharmaceutical compositions comprising an AhR modulator, such as an AhR antagonist of Formula (I) or an enantiomer, diastereomer, racemate, solvate, hydrate, or pharmaceutically acceptable salt thereof, and pharmaceutically acceptable excipients.

In some aspects, pharmaceutical compositions comprising an AhR modulator, such as an AhR antagonist of Formula (I) or an enantiomer, diastereomer, racemate, solvate, hydrate, or pharmaceutically acceptable salt thereof, are provided for use in for modulating constitutive AhR activity in a subject in need thereof.

In some aspects, pharmaceutical compositions comprising an AhR modulator, such as an AhR antagonist of Formula (I) or an enantiomer, diastereomer, racemate, solvate, hydrate, or pharmaceutically acceptable salt thereof, are provided for use in treating a cancer or a cancerous condition by modulating AhR activity.

In some aspects, pharmaceutical compositions comprising an AhR modulator, such as an AhR antagonist of Formula (I) or an enantiomer, diastereomer, racemate, solvate, hydrate, or pharmaceutically acceptable salt thereof, are provided for use in inhibiting proliferation, tissue invasion, metastasis and angiogenesis of cancer cells in a subject having a cancer, a cancerous condition, or a tumor.

In some embodiment, the pharmaceutical composition of the present invention may be for use in inhibiting proliferation, tissue invasion, metastasis and angiogenesis of cancer cells in a subject having a cancer, a cancerous condition, or a tumor.

Pharmaceutical formulations described herein are administrable to a subject in a variety of by multiple administration routes, including but not limited to, oral, parenteral (e.g., intravenous, subcutaneous, intramuscular, rectal, enfometrial or cerebrovascular injection), intranasal, buccal, topical or transdermal administration routes.

In some embodiments, the compounds of Chemical Formula (I) or an enantiomer, diastereomer, racemate, solvate, hydrate, or pharmaceutically acceptable salt thereof are administered orally.

Another aspect of the present invention relates to a method of stimulating the immune system in a patient in need thereof, e.g., in a patient suffering from cancer or an infection (e.g., a viral, bacterial, or parasitic infection). The method includes administering to the patient a therapeutically effective amount of one or a combination of the compounds described herein. In some embodiments, the patient has an increased count of white blood cells, T and/or B lymphocytes, macrophases, dendritic cells, neutrophils, natural killer (NK) cells, and/or platelets after the administering step. In some embodiments, the compound decreases IL-21 level in the patient. The patient may have cancer, or may be immune-compromised.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. Treatment of cancer encompasses inhibiting cancer growth (including causing partial or complete cancer regression), inhibiting cancer progression or metastasis, preventing cancer recurrence or residual disease, and/or prolonging the patient's survival. “A therapeutically effective amount” is an amount of the medication that can achieve the desired curative, palliative, or prophylactic effect for the treated condition.

In some embodiments, the effective dose range of a compound is determined by measuring the patient's blood concentration of the compound under a specified dosing regimen to establish a concentration-time profile, consulting with an established correlation between the concentration-time profiles and effects on cancer inhibition or eradication obtained during a trial, and balancing the therapeutic effects achievable with possible toxicity to the patient, with further consideration of the health condition or physical durability of the patient. The dosing frequency of the compound may be determined similarly. The dosing may be continued until the patiunlessent is free from the cancer.

In some embodiments, an effective amount for tumor therapy may be measured by its ability to stabilize disease progression and/or ameliorate symptoms in a patient, and preferably to reverse disease progression, e.g., by reducing tumor size. In some embodiments, a maintenance dosing may be provided after the patient is free of cancer to ensure its complete elimination or eradication, or prevention of residual disease. The duration of the maintenance dosing can be determined based on clinical trial data.

In some embodiments, a compound may be administered in combination with one or more other cancer therapeutic agents that also target AhR or target molecules other than AhR. Compounds can be formulated either separately from, or together with, the other cancer therapeutic agents. Compounds can be administered either at the same schedule as, or at a different schedule from, the other cancer therapeutic agents. The proportion of a compound relative to other cancer therapeutic agents may be determined by clinical trials. Combining the compounds with the other cancer therapeutic agents may further enhance the efficacy of one another. For example, a compound of the present invention can be administered with an immune checkpoint inhibitor, such as an inhibitor of PD-1, PD-L1 or PD-L2 (e.g., pembrolizumab, nivolumab, or atezolizumab), or administered with CAR-T therapy (e.g., axicabtagene ciloleucel), to achieve additive or synergistic anti-cancer effect.

Dosage regimens may be adjusted to provide the optimum desired response. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of this invention may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values.

It is contemplated that a suitable dose of a compound of the present invention may be in the range of 0.001-200 mg/kg per day, and preferably from about 0.01 mg/kg to about 20 mg/kg body weight per day, such as about 0.5-50 mg/kg, e.g., about 1-20 mg/kg. The compound may for example be administered in a dosage of at least 0.25 mg/kg, e.g., at least 0.5 mg/kg, such as at least 1 mg/kg, e.g., at least 1.5 mg/kg, such as at least 2 mg/kg, e.g., at least 3 mg/kg, such as at least 4 mg/kg, e.g., at least 5 mg/kg; and e.g., up to at most 50 mg/kg, such as up to at the most 30 mg/kg, e.g., up to at the most 20 mg/kg, such as up to at the most 15 mg/kg. Administration will normally be repeated at suitable intervals, e.g., twice a day, thrice a day, once a day, once every week, once every two weeks, or once every three weeks, and for as long as deemed appropriate by the responsible doctor, who may optionally increase or decrease the dosage as necessary.

General Synthetic Methods

The compounds of this invention can be prepared in accordance with one or more of schemes discussed below.

These methods can be used either directly or with obvious variations to trained chemists to prepare key intermediates and certain compounds of this invention.

Suitable synthetic sequences are readily selected per specific structures of this invention, but within the art known to individuals practicing organic synthesis, such as methods summarized in available chemistry data bases, as in CAS Scifinder and Elesevier Reaxys. Based on these general methods, the enablement for making the compounds of this invention is straightforward and can be practiced within a common professional knowledge. Some general synthetic methods to prepare the compounds of this invention are illustrated below in Schemes 1-3 (general procedure A˜C).

One general approach to the compounds of this invention is illustrated in general Scheme 1.

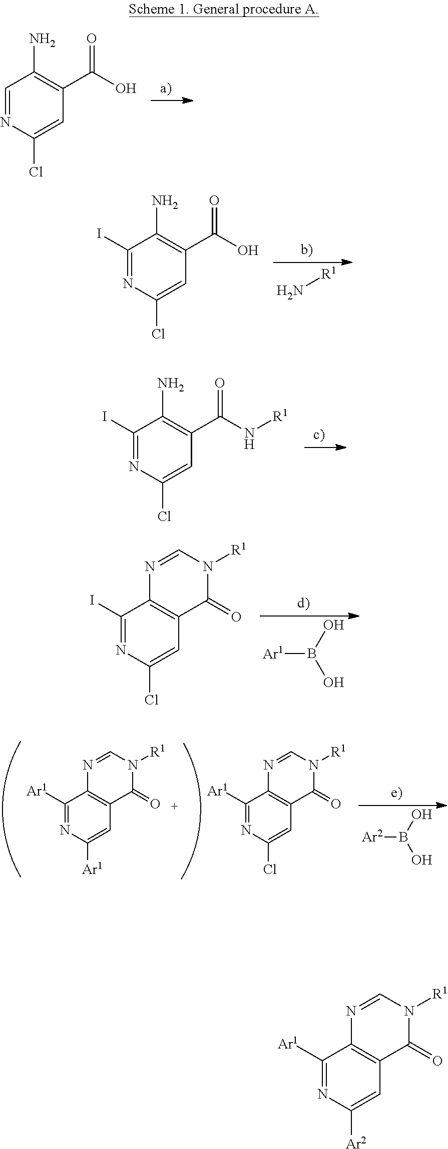

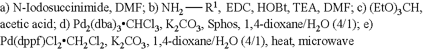

Another general approach to the compounds of this invention is illustrated in general Scheme 2.

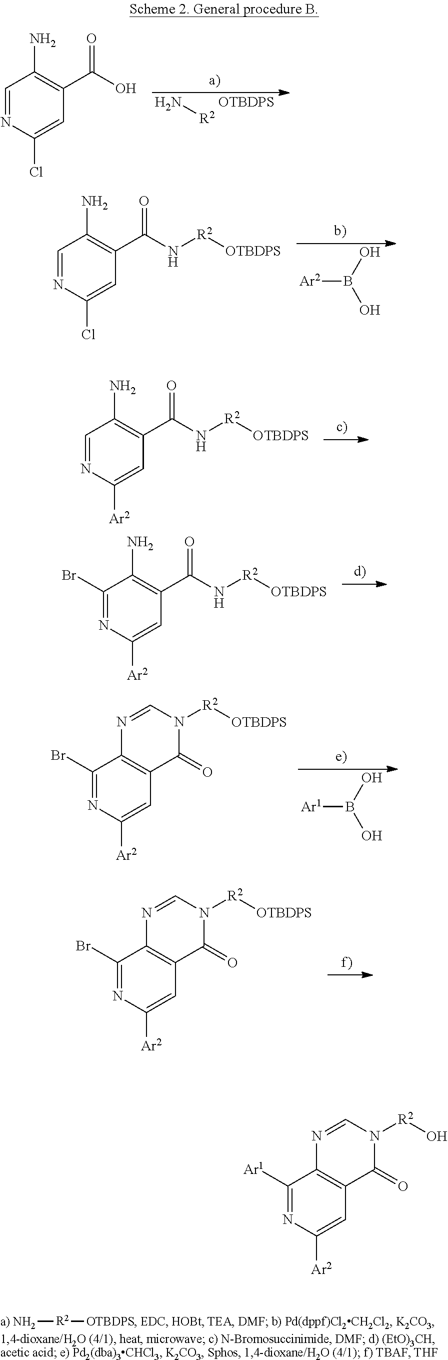

Another general approach to the compounds of this invention is illustrated in general Scheme 3.

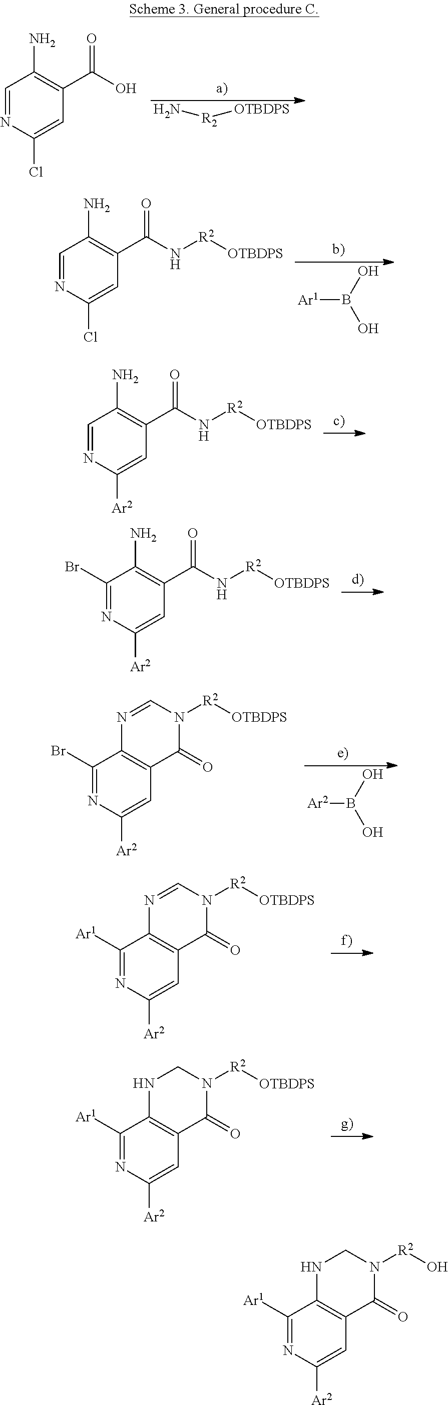

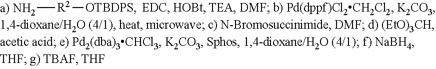

Another general approach to the compounds of this invention is illustrated in general Scheme 4.

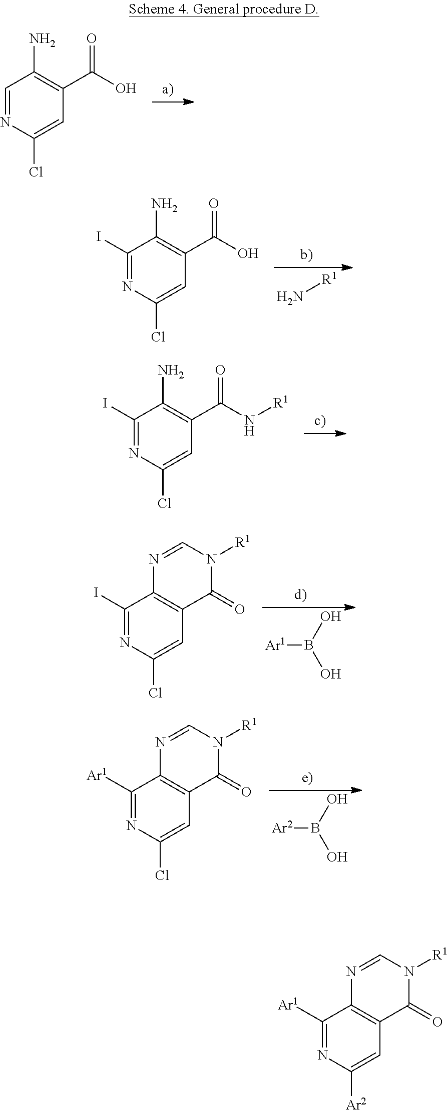

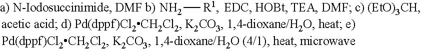

Another general approach to the compounds of this invention is illustrated in general Scheme 5.

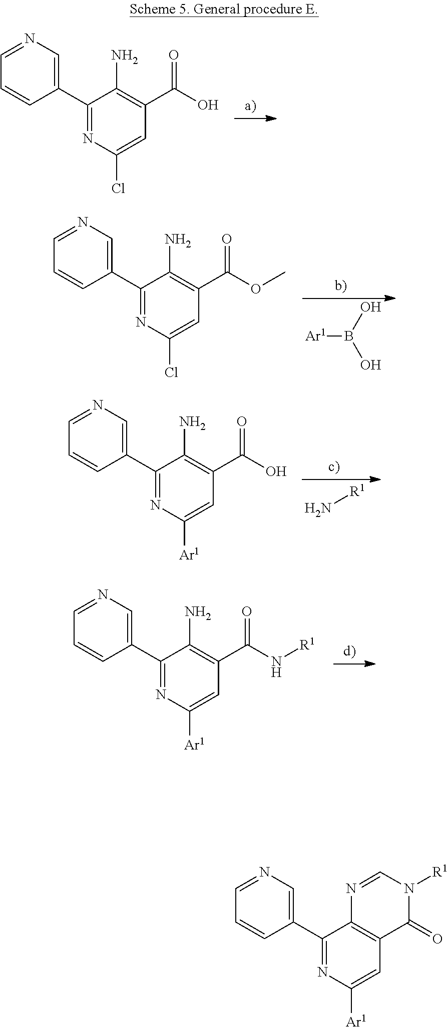

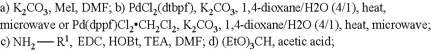

Another general approach to the compounds of this invention is illustrated in general Scheme 6.

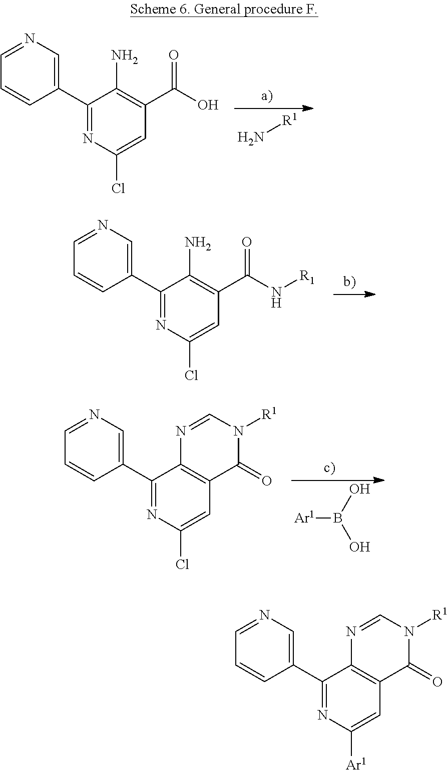

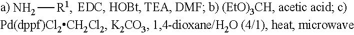

Another general approach to the compounds of this invention is illustrated in general Scheme 7.

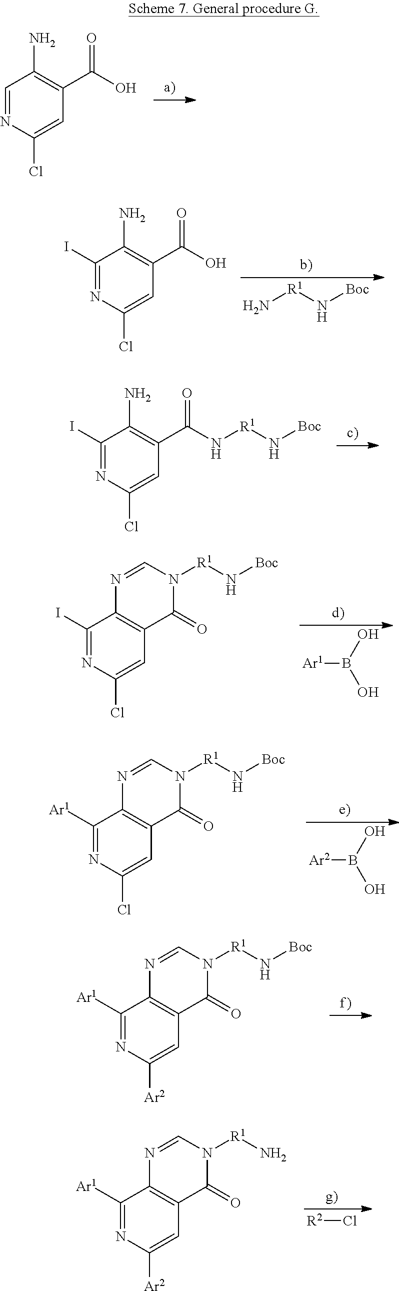

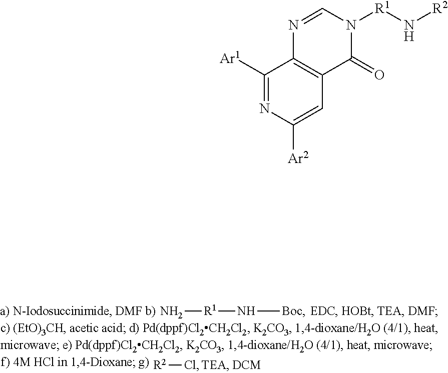

### MODE FOR INVENTION

### Examples

Embodiments of the present invention are described in the following examples, which are meant to illustrate and not limit the scope of this invention. Common abbreviations well known to those with ordinary skills in the synthetic art used throughout.

All chemical reagents were commercially available. Flash column chromatography means silica gel chromatography unless specified otherwise, which was performed on Teledyne Combiflash-RF200 System. ¹H NMR spectra (δ, ppm) are recorded on 400 MHz or 600 MHz instrument. Mass spectroscopy data for a positive ionization method are provided. Preparative HPLC was performed on Agilent technologies G1361A and Gilson Preparative HPLC System.

### Example 1 and 2. 3-(3-hydroxycyclohexyl)-8-(1-methyl-1H-pyrazol-4-yl)-6-(4-(trifluoromethyl)phenyl)pyrido[3,4-d]pyrimidin-4(3H)-one and 3-(3-hydroxycyclohexyl)-6,8-bis(1-methyl-1H-pyrazol-4-yl)pyrido[3,4-d]pyrimidin-4(3H)-one

Scheme for the preparation of the Compound of Example 1:

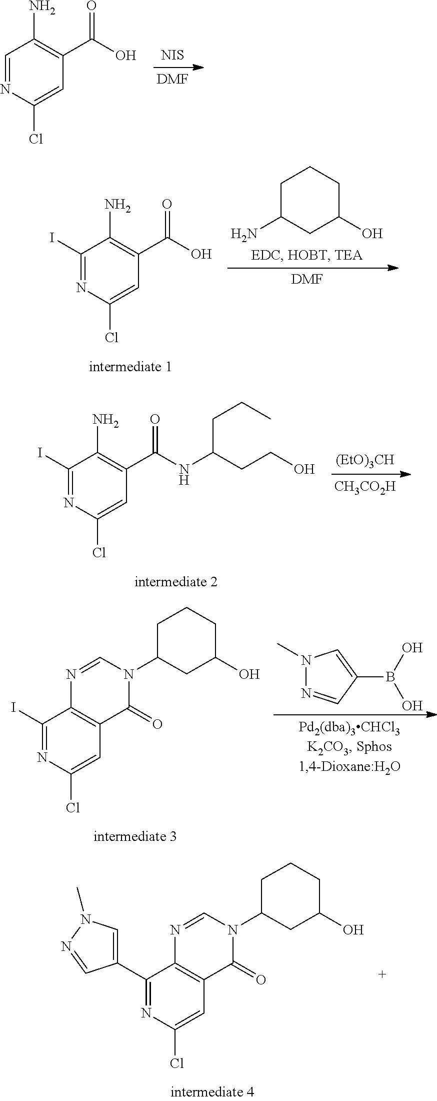

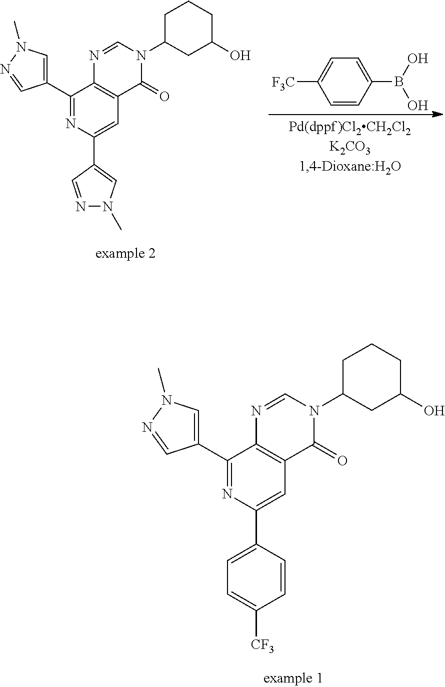 [SMILES: Cn1cc(-c2cc3c(=O)n(C4CCCC(O)C4)cnc3c(-c3cnn(C)c3)n2)cn1 ; OB(O)c1ccc(C(F)(F)F)cc1 ; Cn1cc(-c2nc(-c3ccc(C(F)(F)F)cc3)cc3c(=O)n(C4CCCC(O)C4)cnc23)cn1]

### Intermediate 1. 3-amino-6-chloro-2-iodoisonicotinic acid

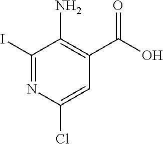 [SMILES: Nc1c(C(=O)O)cc(Cl)nc1I]

5-Amino-2-chloroisonicotinic acid (1) (2 g, 14.5 mmol, 1 equiv.) and N-iodosuccinimide (4.9 g, 21.7 mmol, 1.5 equiv.) were dissolved in DMF (50 mL, 0.3 M) and stirred for 12 h at 100° C. The reaction mixture was diluted with water (50 mL), extracted with EtOAc (50 mL×3), washed with brine (20 mL×3), dried over Na₂SO₄ and glass filtered. The filtrate was evaporated in vacuo to give 6.89 g (80% yield) of 3-amino-6-chloro-2-iodoisonicotinic acid (intermediate 1) and used without further purification.

1H NMR (400 MHz, CDCl₃) δ [ppm]=7.69 (s, 1H); MS (ESI, m/z): 298.90 [M+H]⁺

### Intermediate 2. 3-amino-6-chloro-N-(3-hydroxycyclohexyl)-2-iodoisonicotinamide

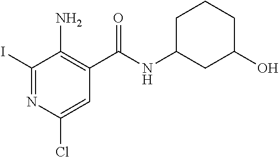 [SMILES: Nc1c(C(=O)NC2CCCC(O)C2)cc(Cl)nc1I]

A mixture of 3-amino-6-chloro-2-iodoisonicotinic acid (intermediate 1) (1.24 g, 4.15 mmol, 1 equiv.), EDC (0.96 g, 4.99 mmol, 1.2 equiv.), HOBT (0.76 g, 4.99 mmol, 1.2 equiv.) and TEA (0.87 mL, 6.23 mmol, 1.5 equiv.) were dissolved in DMF (10 mL, 0.4 M) and stirred for 5 min. Then, 3-aminocyclohexanol (0.53 g, 4.57 mmol, 1.1 equiv.) was added to the reaction mixture and stirred for 12h at 60° C. The reaction mixture was diluted with water (50 mL), extracted with EtOAc (20 mL×3), washed with brine (20 mL×3), dried over Na₂SO₄ and glass filtered. The filtrate was evaporated in vacuo and purified by MPLC (silica gel, 0˜50% EtOAc/Hexane) to give 0.75 g (46% yield) of 3-amino-6-chloro-N-(3-hydroxycyclohexyl)-2-iodoisonicotinamide (intermediate 2).

1H NMR (400 MHz, MeOD) δ [ppm]=7.40 (s, 1H), 3.91 (tt, J=11.5, 3.7 Hz, 1H), 3.66 (tt, J=10.6, 4.1 Hz, 1H), 2.19 (d, J=11.7 Hz, 1H), 1.95 (d, J=12.8 Hz, 1H), 1.87-1.81 (m, 2H), 1.69-1.61 (m, 1H), 1.55-1.50 (m, 1H), 1.40 (ddd, J=12.9, 6.3, 3.2 Hz, 1H). 1.25-1.16 (m, 1H); MS (ESI, m/z): 395.90 [M+H]⁺

### Intermediate 3. 6-chloro-3-(3-hydroxycyclohexyl)-8-iodopyrido[3,4-d]pyrimidin-4(3H)-one

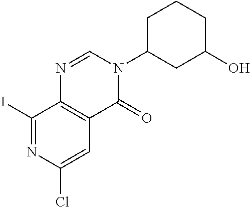 [SMILES: O=c1c2cc(Cl)nc(I)c2ncn1C1CCCC(O)C1]

3-Amino-6-chloro-N-(3-hydroxycyclohexyl)-2-iodoisonicotinamide (intermediate 2) (0.75 g, 1.90 mmol, 1 equiv.) was dissolved in a solution of (EtO)₃CH (2.9 mL, 17.1 mmol, 9 equiv.) and CH₃CO₂H (2.9 mL, 49.8 mmol, 26.2 equiv.). The reaction mixture was stirred and heated in a Biotage microwave initiator at 150° C. for 1 h. The reaction mixture was diluted with water (20 mL), extracted with EtOAc (20 mL×3), washed with brine (20 mL×3), dried over Na₂SO₄ and glass filtered. The filtrate was evaporated in vacuo and purified by MPLC (silica gel, 0˜50% EtOAc/Hexane) to give 0.28 g (36% yield) of 6-chloro-3-(3-hydroxycyclohexyl)-8-iodopyrido[3,4-d]pyrimidin-4(3H)-one (intermediate 3).

1H NMR (400 MHz, CDCl₃) δ [ppm]=8.26 (s, 1H), 8.03 (s, 1H), 4.77 (tt, J=12.1, 3.6 Hz, 1H), 3.90-3.80 (m, 1H), 2.29 (d, J=11.2 Hz, 1H), 2.11 (d, J=11.7 Hz, 1H), 2.04-1.92 (m, 2H), 1.70-1.65 (m, 1H), 1.65-1.58 (m, 1H), 1.53-1.49 (m, 1H), 1.33 (ddd, J=23.8, 12.5, 3.9 Hz, 1H); MS (ESI, m/z): 405.83 [M+H]⁺

### Intermediate 4. and example 2. 6-chloro-3-(3-hydroxycyclohexyl)-8-(1-methyl-1H-pyrazol-4-yl)pyrido[3,4-d]pyrimidin-4(3H)-one and 3-(3-hydroxycyclohexyl)-6,8-bis(1-methyl-1H-pyrazol-4-yl)pyrido[3,4-d]pyrimidin-4(3H)-one

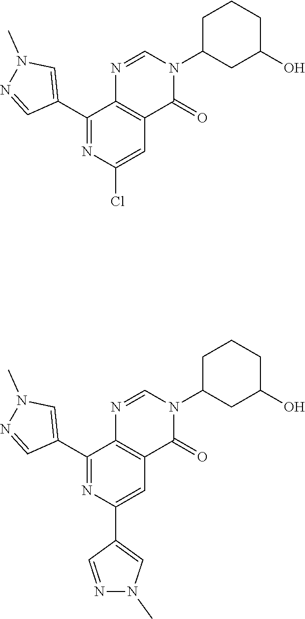 [SMILES: Cn1cc(-c2nc(Cl)cc3c(=O)n(C4CCCC(O)C4)cnc23)cn1 ; Cn1cc(-c2cc3c(=O)n(C4CCCC(O)C4)cnc3c(-c3cnn(C)c3)n2)cn1]

6-Chloro-3-(3-hydroxycyclohexyl)-8-iodopyrido[3,4-d]pyrimidin-4(3H)-one (intermediate 3) (68 mg, 0.168 mmol, 1 equiv.), (1-methyl-1H-pyrazol-4-yl)boronic acid (23.2 mg, 0.184 mmol, 1.1 equiv.), K₂CO₃ (93 mg, 0.671 mmol, 4 equiv.), Sphos (6.9 mg, 0.017 mmol, 0.1 equiv.) and Pd₂(dba)₃.CHCl₃ (8.68 mg, 8.38 μmol, 0.05 equiv.) were dissolved in 1,4-Dioxane/Water (4 mL/1 mL, 0.3 M) and stirred for 12h at 50° C. The reaction mixture was concentrated under reduced pressure and directly subjected to purification by MPLC (silica gel, 0˜10% MeOH/DCM) to give 30 mg (50% yield) of 6-chloro-3-(3-hydroxycyclohexyl)-8-(1-methyl-1H-pyrazol-4-yl)pyrido[3,4-d]pyrimidin-4(3H)-one (intermediate 4) with 13 mg of 3-(3-hydroxycyclohexyl)-6,8-bis(1-methyl-1H-pyrazol-4-yl)pyrido[3,4-d]pyrimidin-4(3H)-one (example 2) in 19% yield.

### Intermediate 4

1H NMR (400 MHz, CDCl₃) δ [ppm]=8.53 (s, 1H), 8.50 (s, 1H), 8.19 (s, 1H), 7.88 (s, 1H), 4.82 (t, J=12.3 Hz, 1H), 3.99 (s, 3H), 3.87 (t, J=10.5 Hz, 1H), 2.31 (d, J=11.0 Hz, 1H), 2.12 (d, J=11.6 Hz, 1H), 2.05-1.94 (m, 2H), 1.79 (s, 1H), 1.74-1.66 (m, 1H), 1.51 (dd, J=18.2, 8.3 Hz, 1H), 1.33 (ddd, J=24.3, 12.7, 3.4 Hz, 1H); MS (ESI, m/z):360.00 [M+H]⁺

### Example 2

1H-NMR (400 MHz, CDCl₃): δ [ppm]=8.57 (s, 1H), 8.52 (s, 1H), 8.15 (s, 1H), 8.10 (s, 1H), 8.06 (s, 1H), 8.00 (s, 1H), 4.90-4.83 (m, 1H), 4.02 (s, 3H), 4.00 (s, 3H), 3.92-3.85 (m, 1H), 2.33 (d, J=11.3 Hz, 1H), 2.13 (d, J=11.5 Hz, 1H), 2.01 (d, J=10.2 Hz, 2H), 1.76-1.67 (m, 2H), 1.64-1.53 (m, 1H), 1.39-1.30 (m, 1H); MS (ESI, m/z):406.07 [M+H]⁺

### Example 1. 3-(3-hydroxycyclohexyl)-8-(1-methyl-1H-pyrazol-4-yl)-6-(4-(trifluoro methyl)phenyl)pyrido[3,4-d]pyrimidin-4(3H)-one

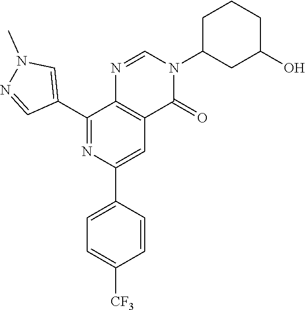 [SMILES: Cn1cc(-c2nc(-c3ccc(C(F)(F)F)cc3)cc3c(=O)n(C4CCCC(O)C4)cnc23)cn1]

6-Chloro-3-(3-hydroxycyclohexyl)-8-(1-methyl-1H-pyrazol-4-yl)pyrido[3,4-d]pyrimidin-4(3H)-one (intermediate 4) (30 mg, 0.083 mmol, 1 equiv.), (4-(trifluoromethyl)phenyl)boronic acid (32 mg, 0.17 mmol, 2 equiv.), K₂CO₃ (35 mg, 0.25 mmol, 3 equiv.) and Pd(dppf)Cl₂.CH₂Cl₂(6.8 mg, 8.34 μmol, 0.1 equiv.) were dissolved in 1,4-Dioxane/Water (4 mL/1 mL, 0.02 M). The reaction mixture was stirred and heated in a Biotage microwave initiator at 130° C. for 30 min. The reaction mixture was concentrated under reduced pressure and directly subjected to purification by MPLC (silica gel Chromatorex NH-DM1020 (NH—SiO₂), 0˜70% EtOAc/Hexane) to give 16 mg (40% yield) of 3-(3-hydroxycyclohexyl)-8-(1-methyl-1H-pyrazol-4-yl)-6-(4-(trifluoromethyl)phenyl)pyrido[3,4-d]pyrimidin-4(3H)-one (example 2).

1H-NMR (400 MHz, CDCl₃): δ [ppm]=8.62 (s, 1H), 8.59 (s, 1H), 8.38 (s, 1H), 8.33 (d, J=8.3 Hz, 2H), 8.24 (s, 1H), 7.78 (d, J=8.3 Hz, 2H), 4.92-4.86 (m, 1H), 4.03 (s, 3H), 3.93-3.86 (m, 1H), 2.35 (d, J=11.6 Hz, 1H), 2.14 (d, J=11.2 Hz, 1H), 2.03 (d, J=10.6 Hz, 2H), 1.78-1.69 (m, 2H), 1.65-1.56 (m, 1H), 1.41-1.31 (m, 1H); MS (ESI, m/z): 470.06 [M+H]⁺

### Example 3. 3-(1-hydroxypropan-2-yl)-6,8-bis(1-methyl-1H-pyrazol-4-yl)pyrido[3,4-d]pyrimidin-4(3H)-one

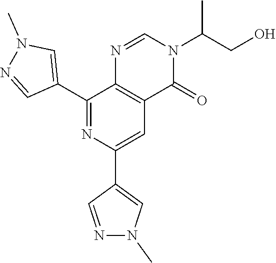 [SMILES: CC(CO)n1cnc2c(-c3cnn(C)c3)nc(-c3cnn(C)c3)cc2c1=O]

Using 2-aminopropan-1-ol, the title compound was obtained as described for the example 2 (Scheme 1. General procedure A.).

1H-NMR (400 MHz, CD₃OD): δ [ppm]=8.62 (s, 1H), 8.40 (s, 1H), 8.28 (s, 1H), 8.15 (s, 1H), 7.99 (s, 1H), 7.86 (s, 1H), 4.93-4.85 (m, 1H), 3.90 (d, J=6.9 Hz, 6H), 3.87 (d, J=6.9 Hz, 1H), 3.77 (dd, J=11.8, 4.3 Hz, 1H), 1.47 (d, J=7.1 Hz, 3H); MS (ESI, m/z): 366.06 [M+H]⁺

### Example 4. 3-(1-hydroxypropan-2-yl)-6-(1-methyl-1H-pyrazol-4-yl)-8-(4-(trifluoromethyl)phenyl)pyrido[3,4-d]pyrimidin-4(3H)-one

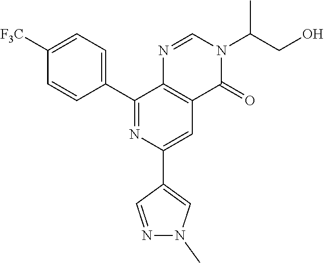 [SMILES: CC(CO)n1cnc2c(-c3ccc(C(F)(F)F)cc3)nc(-c3cnn(C)c3)cc2c1=O]

Using 2-aminopropan-1-ol, the title compound was obtained as described for the example 1 (Scheme 1. General procedure A.).

1H-NMR (400 MHz, CDCl₃): δ [ppm]=8.26-8.16 (m, 2H), 8.04 (dd, J=20.2, 13.9 Hz, 2H), 7.77 (d, J=8.2 Hz, 1H), 7.53 (s, 1H), 7.42 (s, 1H), 5.10-4.99 (m, 1H), 3.96 (d, J=7.4 Hz, 2H), 3.91 (s, 3H), 1.59 (d, J=7.2 Hz, 3H); MS (ESI, m/z): 430.00 [M+H]⁺

### Example 5. 8-(4-chlorophenyl)-3-(1-hydroxypropan-2-yl)-6-(pyridin-3-yl)pyrido[3,4-d]pyrimidin-4(3H)-one

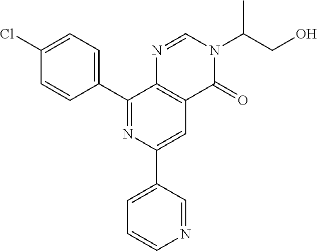 [SMILES: CC(CO)n1cnc2c(-c3ccc(Cl)cc3)nc(-c3cccnc3)cc2c1=O]

Using 2-aminopropan-1-ol, (4-chlorophenyl)boronic acid and pyridin-3-ylboronic acid, the title compound was obtained as described for the example 1 (Scheme 1. General procedure A.).

1H-NMR (400 MHz, CDCl₃): δ [ppm]=9.41 (s, 1H), 8.67 (d, J=3.7 Hz, 1H), 8.56 (s, 1H), 8.50 (d, J=8.1 Hz, 1H), 8.31 (s, 1H), 8.19 (d, J=8.6 Hz, 2H), 7.51 (d, J=8.6 Hz, 2H), 7.47-7.41 (m, 2H), 5.10 (dd, J=11.9, 4.9 Hz, 1H), 4.00 (d, J=4.5 Hz, 1H), 1.60 (d, J=7.3 Hz, 3H); MS (ESI, m/z): 393.02 [M+H]⁺

### Example 6. 3-(1-hydroxypropan-2-yl)-6,8-bis(4-(trifluoromethyl)phenyl)pyrido[3,4-d]pyrimidin-4(3H)-one

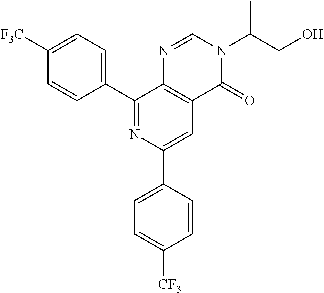 [SMILES: CC(CO)n1cnc2c(-c3ccc(C(F)(F)F)cc3)nc(-c3ccc(C(F)(F)F)cc3)cc2c1=O]

Using 2-aminopropan-1-ol and (4-(trifluoromethyl)phenyl)boronic acid, the title compound was obtained as described for the example 2 (Scheme 1. General procedure A.).

1H-NMR (400 MHz, CD₃OD): δ [ppm]=8.46 (s, 1H), 8.31 (s, 1H), 8.27 (dd, J=7.8, 5.1 Hz, 4H), 7.68 (d, J=8.3 Hz, 4H), 4.94-4.83 (m, 1H), 3.85 (dd, J=11.9, 6.8 Hz, 1H), 3.75 (dd, J=11.9, 4.3 Hz, 1H), 1.44 (d, J=7.1 Hz, 3H); MS (ESI, m/z): 494.04 [M+H]⁺

### Example 7. 2-(6-chloro-8-(4-chlorophenyl)-4-oxopyrido[3,4-d]pyrimidin-3(4H)-yl)propyl acetate

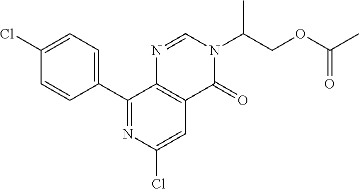 [SMILES: CC(=O)OCC(C)n1cnc2c(-c3ccc(Cl)cc3)nc(Cl)cc2c1=O]

Using 2-aminopropyl acetate and (4-chlorophenyl)boronic acid, the title compound was obtained as described for the intermediate 4 of the example 1 (Scheme 1. General procedure A.).

1H-NMR (400 MHz, CDCl₃): δ [ppm]=8.15 (s, 1H), 8.09 (d, J=8.2 Hz, 2H), 7.48 (d, J=8.5 Hz, 2H), 5.18 (dd, J=11.1, 6.9 Hz, 1H), 4.47-4.32 (m, 2H), 2.04 (s, 3H), 1.59 (d, J=7.2 Hz, 3H); MS (ESI, m/z): 391.95 [M+H]⁺

### Example 8. 3-((1r,4r)-4-hydroxycyclohexyl)-8-(1-methyl-1H-pyrazol-4-yl)-6-(4-(trifluoromethyl)phenyl)pyrido[3,4-d]pyrimidin-4(3H)-one

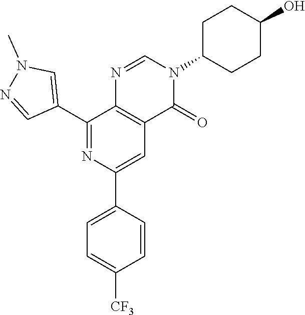 [SMILES: Cn1cc(-c2nc(-c3ccc(C(F)(F)F)cc3)cc3c(=O)n([C@H]4CC[C@H](O)CC4)cnc23)cn1]

Using (1r,4r)-4-aminocyclohexan-1-ol, the title compound was obtained as described for the example 1 (Scheme 1. General procedure A.).

1H-NMR (400 MHz, CDCl₃): δ [ppm]=8.60 (s, 1H), 8.59 (s, 1H), 8.37 (s, 1H), 8.32 (d, J=8.2 Hz, 1H), 8.20 (s, 1H), 7.77 (d, J=8.3 Hz, 1H), 4.88-4.78 (m, 1H), 4.03 (s, 3H), 3.85-3.74 (m, 1H), 2.27-2.19 (m, 2H), 2.15-2.06 (m, 2H), 1.94-1.81 (m, 2H), 1.70-1.57 (m, 2H); MS (ESI, m/z): 470.02 [M+H]⁺

### Example 9. 3-((1r,4r)-4-hydroxycyclohexyl)-6,8-bis(1-methyl-1H-pyrazol-4-yl)pyrido[3,4-d]pyrimidin-4(3H)-one

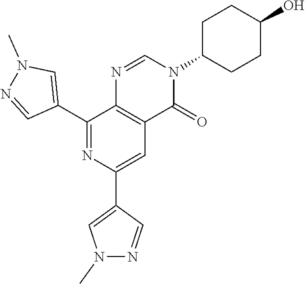 [SMILES: Cn1cc(-c2cc3c(=O)n([C@H]4CC[C@H](O)CC4)cnc3c(-c3cnn(C)c3)n2)cn1]

Using (1r,4r)-4-aminocyclohexan-1-ol, the title compound was obtained as described for the example 2 (Scheme 1. General procedure A.).

1H-NMR (400 MHz, CDCl₃): δ [ppm]=8.55 (s, 1H), 8.52 (s, 1H), 8.11 (s, 1H), 8.10 (s, 1H), 8.05 (s, 1H), 7.99 (s, 1H), 4.87-4.76 (m, 1H), 4.01 (s, 3H), 4.00 (s, 3H), 3.83-3.72 (m, 1H), 2.26-2.16 (m, 2H), 2.11-2.02 (m, 2H), 1.91-1.77 (m, 2H), 1.67-1.56 (m, 2H); MS (ESI, m/z): 406.04 [M+H]⁺

### Example 10. 6-(4-chlorophenyl)-3-((1r,4r)-4-hydroxycyclohexyl)-8-(1-methyl-1H-pyrazol-4-yl)pyrido[3,4-d]pyrimidin-4(3H)-one

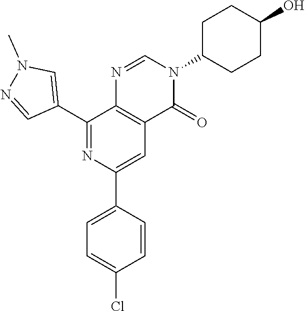

Using (1r,4r)-4-aminocyclohexan-1-ol and (4-chlorophenyl)boronic acid, the title compound was obtained as described for the example 1 (Scheme 1. General procedure A.).

1H-NMR (400 MHz, CDCl₃): δ [ppm]=8.59 (s, 1H), 8.58 (s, 1H), 8.31 (s, 1H), 8.18 (s, 1H), 8.16 (d, J=8.8 Hz, 2H), 7.49 (d, J=8.6 Hz, 2H), 4.88-4.78 (m, 1H), 4.02 (s, 3H), 3.84-3.74 (m, 1H), 2.26-2.18 (m, 2H), 2.14-2.02 (m, 2H), 1.93-1.80 (m, 2H), 1.70-1.61 (m, 2H); MS (ESI, m/z): 436.01 [M+H]⁺

### Example 11. 3-(2-hydroxypropyl)-6,8-bis(1-methyl-1H-pyrazol-4-yl)pyrido[3,4-d]pyrimidin-4(3H)-one

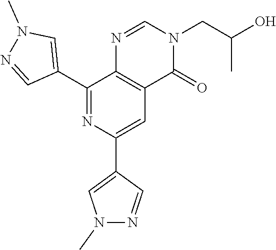 [SMILES: CC(O)Cn1cnc2c(-c3cnn(C)c3)nc(-c3cnn(C)c3)cc2c1=O]

Using 1-aminopropan-2-ol, the title compound was obtained as described for the example 2 (Scheme 1. General procedure A.).

1H-NMR (400 MHz, CD₃OD): δ [ppm]=8.75 (s, 1H), 8.51 (s, 1H), 8.29 (s, 1H), 8.26 (s, 1H), 8.11 (s, 1H), 8.01 (s, 1H), 4.28 (dd, J=13.6, 3.0 Hz, 1H), 4.19-4.11 (m, 1H), 4.00 (s, 3H), 3.99 (s, 3H), 3.74 (dd, J=13.6, 8.8 Hz, 1H), 1.30 (d, J=6.3 Hz, 3H); MS (ESI, m/z): 366.01 [M+H]⁺

### Example 12. 3-(2-hydroxypropyl)-8-(1-methyl-1H-pyrazol-4-yl)-6-(4-(trifluoromethyl)phenyl)pyrido[3,4-d]pyrimidin-4(3H)-one

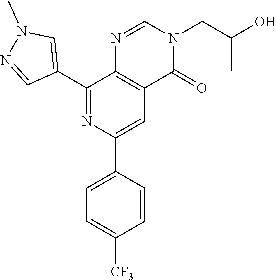 [SMILES: CC(O)Cn1cnc2c(-c3cnn(C)c3)nc(-c3ccc(C(F)(F)F)cc3)cc2c1=O]

Using 1-aminopropan-2-ol, the title compound was obtained as described for the example 1 (Scheme 1. General procedure A.).

1H-NMR (400 MHz, CDCl₃): δ [ppm]=8.50 (s, 1H), 8.49 (s, 1H), 8.32 (s, 1H), 8.24 (d, J=8.2 Hz, 2H), 8.18 (s, 1H), 7.72 (d, J=8.3 Hz, 2H), 4.38 (dd, J=13.6, 2.5 Hz, 1H), 4.30 (br, 1H), 3.99 (s, 3H), 3.70 (dd, J=13.6, 8.5 Hz, 1H), 2.78 (d, J=4.4 Hz, 1H), 1.38 (d, J=6.3 Hz, 3H); MS (ESI, m/z): 430.00 [M+H]⁺

### Example 13. 6-(4-chlorophenyl)-3-(2-hydroxypropyl)-8-(1-methyl-1H-pyrazol-4-yl)pyrido[3,4-d]pyrimidin-4(3H)-one

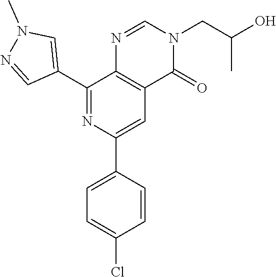 [SMILES: CC(O)Cn1cnc2c(-c3cnn(C)c3)nc(-c3ccc(Cl)cc3)cc2c1=O]

Using 1-aminopropan-2-ol and (4-chlorophenyl)boronic acid, the title compound was obtained as described for the example 1 (Scheme 1. General procedure A.).

1H-NMR (400 MHz, CDCl₃): δ [ppm]=8.55 (s, 1H), 8.53 (s, 1H), 8.28 (s, 1H), 8.17 (s, 1H), 8.12 (d, J=8.6 Hz, 2H), 7.47 (d, J=8.5 Hz, 2H), 4.36 (dd, J=13.7, 2.6 Hz, 1H), 4.28 (br, 1H), 4.00 (s, 3H), 3.73 (dd, J=13.6, 8.3 Hz, 1H), 2.43 (d, J=4.5 Hz, 1H), 1.37 (d, J=6.3 Hz, 3H); MS (ESI, m/z): 396.00 [M+H]⁺

### Example 14. 3-(2-hydroxypropyl)-8-(1-methyl-1H-pyrazol-4-yl)-6-(6-(trifluoromethyl)pyridin-3-yl)pyrido[3,4-d]pyrimidin-4(3H)-one

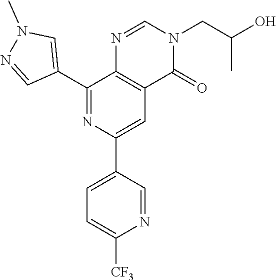 [SMILES: CC(O)Cn1cnc2c(-c3cnn(C)c3)nc(-c3ccc(C(F)(F)F)nc3)cc2c1=O]

Using 1-aminopropan-2-ol and (6-(trifluoromethyl)pyridin-3-yl)boronic acid, the title compound was obtained as described for the example 1 (Scheme 1. General procedure A.).

1H-NMR (400 MHz, CDCl₃): δ [ppm]=9.48 (s, 1H), 8.62 (m, 1H), 8.59 (s, 1H), 8.52 (s, 1H), 8.36 (s, 1H), 8.25 (s, 1H), 7.81 (d, J=8.2 Hz, 1H), 4.39 (dd, J=13.5, 2.5 Hz, 1H), 4.30 (br, 1H), 4.01 (s, 3H), 3.73 (dd, J=13.6, 8.4 Hz, 1H), 2.41 (d, J=4.2 Hz, 1H), 1.39 (d, J=6.3 Hz, 3H); MS (ESI, m/z): 431.00 [M+H]⁺

### Example 15. 3-((1S,2R)-2-hydroxycyclohexyl)-8-(pyridin-3-yl)-6-(4-(trifluoromethyl)phenyl)pyrido[3,4-d]pyrimidin-4(3H)-one

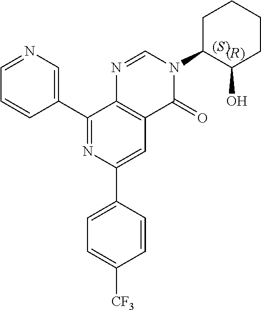

Using (1R,2S)-2-aminocyclohexanol, pyridin-3-ylboronic acid and (4-(trifluoromethyl)phenyl)boronic acid, the title compound was obtained as described for the example 1 (Scheme 1. General procedure A.).

1H-NMR (400 MHz, MeOD): δ [ppm]=9.42 (s, 1H), 8.73 (d, J=8.0 Hz, 1H), 8.64 (d, J=4.8 Hz, 1H), 8.62 (s, 1H), 8.51 (s, 1H), 8.41 (d, J=8.2 Hz, 2H), 7.83 (d, J=8.3 Hz, 2H), 7.61 (dd, J=7.9, 4.9 Hz, 1H), 4.90-4.85 (m, 1H), 4.09 (s, 1H), 2.41-2.27 (m, 1H), 2.06-1.93 (m, 2H), 1.76 (d, J=9.3 Hz, 2H), 1.71 (s, 1H), 1.59 (dt, J=17.5, 8.6 Hz, 2H); MS (ESI, m/z): 467.15 [M+H]⁺

### Example 16. 3-((1R,2S)-2-hydroxycyclohexyl)-8-(pyridin-3-yl)-6-(4-(trifluoromethyl)phenyl)pyrido[3,4-d]pyrimidin-4(3H)-one

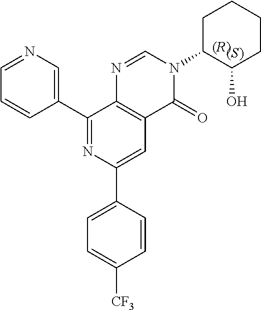

Using (1S,2R)-2-aminocyclohexanol hydrochloride, pyridin-3-ylboronic acid and (4-(trifluoromethyl)phenyl)boronic acid, the title compound was obtained as described for the example 1 (Scheme 1. General procedure A.).

1H-NMR (400 MHz, MeOD): δ [ppm]=9.41 (s, 1H), 8.75-8.66 (m, 1H), 8.63 (d, J=4.7 Hz, 1H), 8.58 (d, J=6.5 Hz, 1H), 8.49 (d, J=1.9 Hz, 1H), 8.38 (dd, J=8.1, 3.9 Hz, 2H), 7.81 (d, J=6.8 Hz, 2H), 7.63 (d, J=8.9 Hz, 1H), 7.61-7.57 (m, 1H), 4.86 (dd, J=13.3, 2.6 Hz, 1H), 4.08 (s, 1H), 2.34 (dd, J=22.2, 12.3 Hz, 1H), 2.04-1.93 (m, 2H), 1.77 (t, J=9.3 Hz, 2H), 1.71 (d, J=10.1 Hz, 1H), 1.64-1.51 (m, 2H); MS (ESI, m/z): 467.13 [M+H]⁺

### Example 17. 3-((1S,2R)-2-hydroxycyclohexyl)-8-(1-methyl-1H-pyrazol-4-yl)-6-(4-(trifluoromethyl)phenyl)pyrido[3,4-d]pyrimidin-4(3H)-one

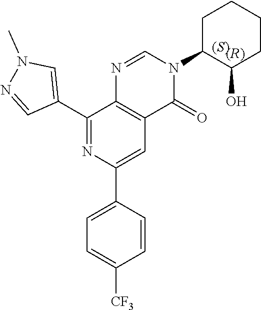

Using (1R,2S)-2-aminocyclohexanol, (1-methyl-1H-pyrazol-4-yl)boronic acid and (4-(trifluoromethyl)phenyl)boronic acid, the title compound was obtained as described for the example 1 (Scheme 1. General procedure A.).

1H-NMR (400 MHz, CDCl3): δ [ppm]=8.48 (s, 1H), 8.37 (s, 1H), 8.33 (s, 1H), 8.27 (s, 1H), 8.14 (d, J=8.1 Hz, 2H), 7.67 (d, J=8.3 Hz, 2H), 4.95 (dd, J=10.2, 2.4 Hz, 1H), 4.30 (s, 1H), 3.99 (s, 3H), 3.23 (s, 1H), 2.39-2.27 (m, 1H), 2.03 (d, J=12.2 Hz, 2H), 1.89-1.74 (m, 2H), 1.68 (dd, J=24.7, 13.2 Hz, 3H); MS (ESI, m/z): 470.18 [M+H]⁺

### Example 18. 3-((1R,2S)-2-hydroxycyclohexyl)-8-(1-methyl-1H-pyrazol-4-yl)-6-(4-(trifluoromethyl)phenyl)pyrido[3,4-d]pyrimidin-4(3H)-one

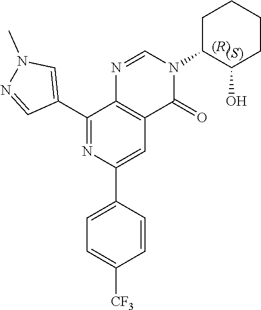

Using (1S,2R)-2-aminocyclohexanol hydrochloride, (1-methyl-1H-pyrazol-4-yl)boronic acid and (4-(trifluoromethyl)phenyl)boronic acid, the title compound was obtained as described for the example 1 (Scheme 1. General procedure A.).

1H-NMR (400 MHz, CDCl3): δ [ppm]=8.50 (s, 1H), 8.37 (d, J=9.4 Hz, 2H), 8.28 (s, 1H), 8.17 (d, J=8.2 Hz, 2H), 7.69 (d, J=8.2 Hz, 2H), 4.96 (d, J=13.3 Hz, 1H), 4.29 (s, 1H), 3.99 (s, 3H), 3.06 (s, 1H), 2.34 (dd, J=22.1, 12.5 Hz, 1H), 2.06-1.98 (m, 2H), 1.90-1.78 (m, 2H), 1.75-1.63 (m, 3H); MS (ESI, m/z): 470.18 [M+H]⁺

### Example 19. 3-((1R,2S)-2-hydroxycyclohexyl)-8-(1-methyl-1H-pyrazol-4-yl)-6-(4-(trifluoromethyl)phenyl)pyrido[3,4-d]pyrimidin-4(3H)-one

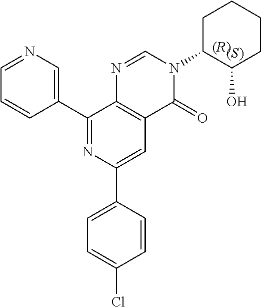

Using (1S,2R)-2-aminocyclohexanol hydrochloride, pyridin-3-ylboronic acid and (4-chlorophenyl)boronic acid, the title compound was obtained as described for the example 1 (Scheme 1. General procedure A.).

1H-NMR (400 MHz, CDCl3): δ [ppm]=9.34 (d, J=1.6 Hz, 1H), 8.60 (dd, J=4.9, 1.5 Hz, 1H), 8.48-8.43 (m, 1H), 8.43 (s, 1H), 8.40 (s, 1H), 8.07 (d, J=8.6 Hz, 2H), 7.45 (d, J=8.6 Hz, 2H), 7.38 (dd, J=7.7, 4.9 Hz, 1H), 4.93 (d, J=12.8 Hz, 1H), 4.19 (s, 1H), 3.09 (s, 1H), 2.32 (ddd, J=25.3, 12.6, 3.5 Hz, 1H), 2.06-1.96 (m, 2H), 1.81 (dd, J=25.2, 12.1 Hz, 2H), 1.71 (d, J=11.0 Hz, 2H), 1.63 (d, J=17.4 Hz, 1H); MS (ESI, m/z): 433.14 [M+H]⁺

### Example 20. 6-(4-chlorophenyl)-3-((1S,2R)-2-hydroxycyclohexyl)-8-(1-methyl-1H-pyrazol-4-yl)pyrido[3,4-d]pyrimidin-4(3H)-one

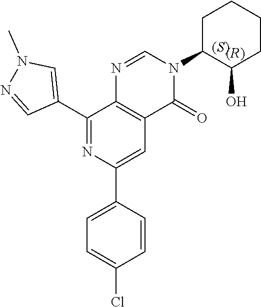

Using (1R,2S)-2-aminocyclohexanol, (1-methyl-1H-pyrazol-4-yl)boronic acid and (4-chlorophenyl)boronic acid, the title compound was obtained as described for the example 1 (Scheme 1. General procedure A.).

1H-NMR (400 MHz, CDCl3): δ [ppm]=8.48 (s, 1H), 8.37 (d, J=9.2 Hz, 2H), 8.21 (s, 1H), 8.03 (d, J=8.6 Hz, 2H), 7.42 (d, J=8.6 Hz, 2H), 4.94 (d, J=13.0 Hz, 1H), 4.27 (s, 1H), 2.85 (d, J=4.2 Hz, 1H), 2.33 (dt, J=21.6, 10.7 Hz, 1H), 2.06-1.94 (m, 2H), 1.89-1.75 (m, 2H), 1.75-1.66 (m, 2H), 1.63 (d, J=16.2 Hz, 1H); MS (ESI, m/z): 436.15 [M+H]⁺

### Example 21. 6-(4-chlorophenyl)-3-((1S,2R)-2-hydroxycyclohexyl)-8-(pyridin-3-yl)pyrido[3,4-d]pyrimidin-4(3H)-one

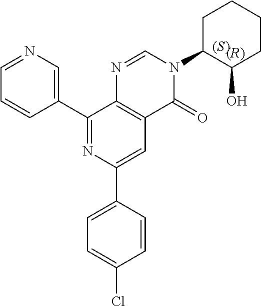

Using (1R,2S)-2-aminocyclohexanol, pyridin-3-ylboronic acid and (4-chlorophenyl)boronic acid, the title compound was obtained as described for the example 1 (Scheme 1. General procedure A.).

1H-NMR (400 MHz, CDCl3): δ [ppm]=9.33 (d, J=1.8 Hz, 1H), 8.59 (dd, J=4.8, 1.6 Hz, 1H), 8.47-8.43 (m, 1H), 8.42 (s, 1H), 8.38 (s, 1H), 8.05 (d, J=8.6 Hz, 2H), 7.44 (d, J=8.6 Hz, 2H), 4.93 (d, J=12.7 Hz, 1H), 4.18 (s, 1H), 3.31 (s, 1H), 2.38-2.26 (m, 1H), 2.06-1.95 (m, 2H), 1.87-1.75 (m, 2H), 1.75-1.67 (m, 2H), 1.63 (d, J=13.8 Hz, 1H); MS (ESI, m/z): 433.17 [M+H]⁺

### Example 22. 8-(1-methyl-1H-pyrazol-4-yl)-3-(3,3,3-trifluoro-2-hydroxypropyl)-6-(4-(trifluoromethyl)phenyl)pyrido[3,4-d]pyrimidin-4(3H)-one

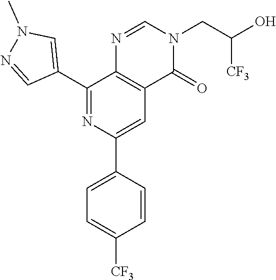 [SMILES: Cn1cc(-c2nc(-c3ccc(C(F)(F)F)cc3)cc3c(=O)n(CC(O)C(F)(F)F)cnc23)cn1]

Using 3-amino-1,1,1-trifluoropropan-2-ol, (1-methyl-1H-pyrazol-4-yl)boronic acid and (4-(trifluoromethyl)phenyl)boronic acid, the title compound was obtained as described for the example 1 (Scheme 1. General procedure A.).

1H-NMR (400 MHz, CDCl3): δ [ppm]=8.34 (s, 1H), 8.25 (s, 1H), 8.24 (s, 1H), 8.10 (s, 1H), 8.09 (s, 2H), 7.65 (d, J=8.3 Hz, 2H), 5.44 (s, 1H), 4.71 (dd, J=13.7, 2.3 Hz, 1H), 4.67-4.56 (m, 1H), 3.95 (s, 3H), 3.83 (dd, J=13.7, 9.5 Hz, 1H); MS (ESI, m/z): 484.14 [M+H]⁺

### Example 23. 6-(4-chlorophenyl)-8-(1-methyl-1H-pyrazol-4-yl)-3-(3,3,3-trifluoro-2-hydroxypropyl)pyrido[3,4-d]pyrimidin-4(3H)-one

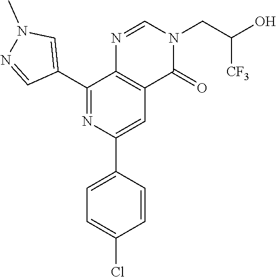 [SMILES: Cn1cc(-c2nc(-c3ccc(Cl)cc3)cc3c(=O)n(CC(O)C(F)(F)F)cnc23)cn1]

Using 3-amino-1,1,1-trifluoropropan-2-ol, (1-methyl-1H-pyrazol-4-yl)boronic acid and (4-chlorophenyl)boronic acid, the title compound was obtained as described for the example 1 (Scheme 1. General procedure A.).

1H-NMR (400 MHz, CDCl3): δ [ppm]=8.31 (s, 1H), 8.17 (s, 1H), 8.13 (s, 1H), 8.04 (s, 1H), 7.91 (d, J=8.5 Hz, 2H), 7.37 (d, J=8.4 Hz, 2H), 5.63 (s, 1H), 4.68 (d, J=13.6 Hz, 1H), 4.66-4.56 (m, 1H), 3.92 (s, 2H), 3.79 (dd, J=13.5, 9.4 Hz, 1H); MS (ESI, m/z): 450.10 [M+H]⁺

### Example 24. 6-(4-chlorophenyl)-8-(pyridin-3-yl)-3-(3,3,3-trifluoro-2-hydroxypropyl)pyrido[3,4-d]pyrimidin-4(3H)-one

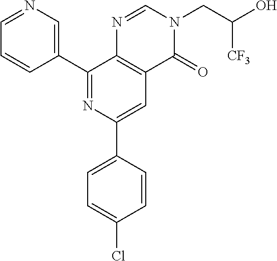 [SMILES: O=c1c2cc(-c3ccc(Cl)cc3)nc(-c3cccnc3)c2ncn1CC(O)C(F)(F)F]

Using 3-amino-1,1,1-trifluoropropan-2-ol, pyridin-3-ylboronic acid and (4-chlorophenyl)boronic acid, the title compound was obtained as described for the example 1 (Scheme 1. General procedure A.).

1H-NMR (400 MHz, CDCl3): δ [ppm]=δ 9.21 (d, J=1.5 Hz, 1H), 8.42 (dd, J=4.9, 1.4 Hz, 1H), 8.40-8.35 (m, 1H), 8.18 (d, J=8.1 Hz, 2H), 7.87 (d, J=8.6 Hz, 2H), 7.38 (d, J=8.6 Hz, 2H), 7.36-7.32 (m, 1H), 4.76 (d, J=13.5 Hz, 1H), 4.70-4.55 (m, 1H), 3.75 (dd, J=13.5, 10.1 Hz, 1H); MS (ESI, m/z): 447.09 [M+H]⁺

### Example 25. 8-(pyridin-3-yl)-3-(3,3,3-trifluoro-2-hydroxypropyl)-6-(4-(trifluoromethyl)phenyl)pyrido[3,4-d]pyrimidin-4(3H)-one

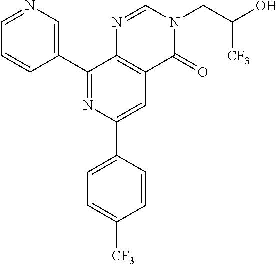 [SMILES: O=c1c2cc(-c3ccc(C(F)(F)F)cc3)nc(-c3cccnc3)c2ncn1CC(O)C(F)(F)F]

Using 3-amino-1,1,1-trifluoropropan-2-ol, pyridin-3-ylboronic acid and (4-(trifluoromethyl)phenyl)boronic acid, the title compound was obtained as described for the example 1 (Scheme 1. General procedure A.).

1H-NMR (400 MHz, CDCl3): δ [ppm]=9.29 (d, J=1.6 Hz, 1H), 8.48 (dd, J=4.9, 1.5 Hz, 1H), 8.45-8.39 (m, 1H), 8.29 (s, 1H), 8.23 (s, 1H), 8.08 (d, J=8.2 Hz, 2H), 7.67 (d, J=8.3 Hz, 2H), 7.39 (dd, J=8.0, 4.9 Hz, 1H), 4.77 (dd, J=13.6, 2.5 Hz, 1H), 4.71-4.62 (m, 1H), 3.81 (dd, J=13.5, 9.9 Hz, 1H); MS (ESI, m/z): 480.90 [M+H]⁺

### Example 26. 6-(4-chlorophenyl)-3-(3-hydroxyphenyl)-8-(1-methyl-1H-pyrazol-4-yl)pyrido[3,4-d]pyrimidin-4(3H)-one

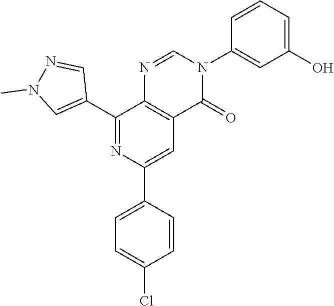 [SMILES: Cn1cc(-c2nc(-c3ccc(Cl)cc3)cc3c(=O)n(-c4cccc(O)c4)cnc23)cn1]

Using 3-aminophenol, (1-methyl-1H-pyrazol-4-yl)boronic acid and (4-chlorophenyl)boronic acid, the title compound was obtained as described for the example 1 (Scheme 1. General procedure A.).

1H NMR (400 MHz, CDCl3): δ [ppm]=8.62 (s, 1H), 8.62 (s, 1H), 8.35 (s, 1H), 8.19 (s, 1H), 8.17 (d, J=8.5 Hz, 2H), 7.49 (d, J=8.5 Hz, 2H), 7.47-7.42 (m, 1H), 7.04-6.93 (m, 3H), 5.83 (br, 1H), 4.04 (s, 3H); MS (ESI, m/z): 430.08 [M+H]⁺

### Example 27. 3-(3-hydroxyphenyl)-8-(1-methyl-1H-pyrazol-4-yl)-6-(4-(trifluoromethyl)phenyl)pyrido[3,4-d]pyrimidin-4(3H)-one

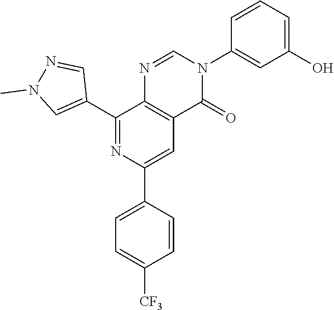 [SMILES: Cn1cc(-c2nc(-c3ccc(C(F)(F)F)cc3)cc3c(=O)n(-c4cccc(O)c4)cnc23)cn1]

Using 3-aminophenol, (1-methyl-1H-pyrazol-4-yl)boronic acid and (4-(trifluoromethyl)phenyl)boronic acid, the title compound was obtained as described for the example 1 (Scheme 1. General procedure A.).

1H NMR (400 MHz, MeOD): δ [ppm]=8.80 (s, 1H), 8.58 (s, 1H), 8.47-8.33 (m, 4H), 7.82 (d, J=8.3 Hz, 2H), 7.45-7.38 (m, 1H), 7.05-6.92 (m, 3H), 4.00 (s, 3H); MS (ESI, m/z): 464.1 [M+H]⁺

### Example 28. 6-(4-chlorophenyl)-3-((1R,3S)-3-hydroxycyclopentyl)-8-(pyridin-3-yl)pyrido[3,4-d]pyrimidin-4(3H)-one

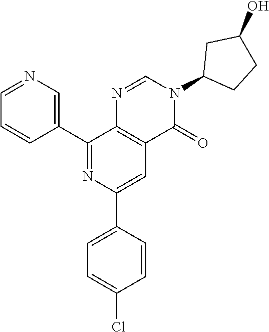 [SMILES: O=c1c2cc(-c3ccc(Cl)cc3)nc(-c3cccnc3)c2ncn1[C@@H]1CC[C@H](O)C1]

Using (1S,3R)-3-aminocyclopentanol, pyridin-3-ylboronic acid and (4-chlorophenyl)boronic acid, the title compound was obtained as described for the example 1 (Scheme 1. General procedure A.).

1H NMR (400 MHz, CDCl3): δ [ppm]=9.46 (d, J=1.8 Hz, 1H), 8.71 (dd, J=4.8, 1.6 Hz, 1H), 8.60 (s, 1H), 8.56 (dd, J=1.9, 1.9 Hz, 1H), 8.54 (s, 1H), 8.19 (d, J=8.6 Hz, 2H), 7.49 (d, J=8.5 Hz, 2H), 7.48-7.44 (m, 1H), 5.33-5.23 (m, 1H), 4.58-4.48 (m, 1H), 2.84 (br, 1H), 2.59-2.49 (m, 1H), 2.40-2.31 (m, 1H), 2.29-2.18 (m, 1H), 2.07-1.95 (m, 2H), 1.88-1.78 (m, 1H); MS (ESI, m/z): 419.1 [M+H]⁺

### Example 29. 3-((1R,3S)-3-hydroxycyclopentyl)-8-(pyridin-3-yl)-6-(4-(trifluoromethyl)phenyl)pyrido[3,4-d]pyrimidin-4(3H)-one

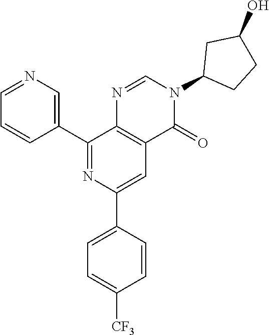 [SMILES: O=c1c2cc(-c3ccc(C(F)(F)F)cc3)nc(-c3cccnc3)c2ncn1[C@@H]1CC[C@H](O)C1]

Using (1S,3R)-3-aminocyclopentanol, pyridin-3-ylboronic acid and (4-(trifluoromethyl)phenyl)boronic acid, the title compound was obtained as described for the example 1 (Scheme 1. General procedure A.).

1H NMR (400 MHz, CDCl3): δ [ppm]=9.48 (s, 1H), 8.74 (d, J=3.9 Hz, 1H), 8.64 (s, 1H), 8.62 (s, 1H), 8.58 (d, J=8.1 Hz, 1H), 8.36 (d, J=8.2 Hz, 2H), 7.78 (d, J=8.3 Hz, 2H), 7.53-7.46 (m, 1H), 5.35-5.26 (m, 1H), 4.57-4.51 (m, 1H), 2.72 (br, 1H), 2.60-2.51 (m, 1H), 2.43-2.33 (m, 1H), 2.29-2.20 (m, 1H), 2.05-1.98 (m, 2H), 1.89-1.79 (m, 1H); MS (ESI, m/z): 453.15 [M+H]⁺

### Example 30. 6-(4-chlorophenyl)-3-((1R,3S)-3-hydroxycyclopentyl)-8-(1-methyl-1H-pyrazol-4-yl)pyrido[3,4-d]pyrimidin-4(3H)-one

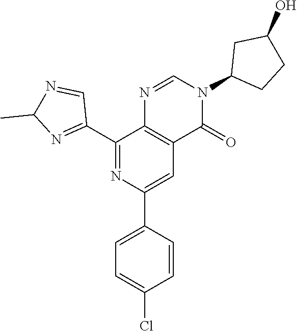 [SMILES: CC1N=CC(c2nc(-c3ccc(Cl)cc3)cc3c(=O)n([C@@H]4CC[C@H](O)C4)cnc23)=N1]

Using (1S,3R)-3-aminocyclopentanol, (1-methyl-1H-pyrazol-4-yl)boronic acid and (4-chlorophenyl)boronic acid, the title compound was obtained as described for the example 1 (Scheme 1. General procedure A.).

1H NMR (400 MHz, CDCl3): δ [ppm]=8.64 (s, 1H), 8.60 (s, 1H), 8.57 (s, 1H), 8.33 (s, 1H), 8.18 (d, J=8.5 Hz, 2H), 7.50 (d, J=8.5 Hz, 2H), 5.29-5.20 (m, 1H), 4.58-4.51 (m, 1H), 4.04 (s, 3H), 2.61-2.51 (m, 1H), 2.39-2.22 (m, 2H), 2.10-1.97 (m, 2H), 1.90-1.79 (m, 1H); MS (ESI, m/z): 422.15 [M+H]⁺

### Example 31. 3-((1R,3S)-3-hydroxycyclopentyl)-8-(1-methyl-1H-pyrazol-4-yl)-6-(4-(trifluoromethyl)phenyl)pyrido[3,4-d]pyrimidin-4(3H)-one

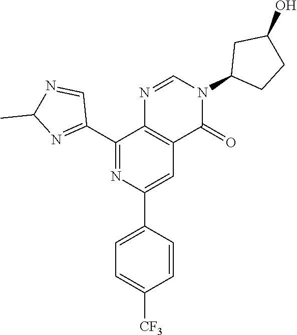 [SMILES: CC1N=CC(c2nc(-c3ccc(C(F)(F)F)cc3)cc3c(=O)n([C@@H]4CC[C@H](O)C4)cnc23)=N1]

Using (1S,3R)-3-aminocyclopentanol, (1-methyl-1H-pyrazol-4-yl)boronic acid and (4-(trifluoromethyl)phenyl)boronic acid, the title compound was obtained as described for the example 1 (Scheme 1. General procedure A.).

1H NMR (400 MHz, CDCl3): δ [ppm]=8.66 (s, 1H), 8.62 (s, 1H), 8.61 (s, 1H), 8.40 (s, 1H), 8.35 (d, J=8.1 Hz, 2H), 7.79 (d, J=8.2 Hz, 2H), 5.33-5.24 (m, 1H), 4.58-4.52 (m, 1H), 4.05 (s, 3H), 2.83-2.75 (m, 1H), 2.63-2.53 (m, 1H), 2.41-2.33 (m, 1H), 2.31-2.22 (m, 1H), 2.11-2.02 (m, 1H), 1.91-1.81 (m, 1H); MS (ESI, m/z): 456.18 [M+H]⁺

### Example 32. 6-(4-chlorophenyl)-3-((1S,3R)-3-hydroxycyclopentyl)-8-(pyridin-3-yl)pyrido[3,4-d]pyrimidin-4(3H)-one

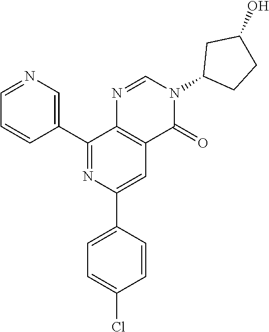 [SMILES: O=c1c2cc(-c3ccc(Cl)cc3)nc(-c3cccnc3)c2ncn1[C@H]1CC[C@@H](O)C1]

Using (1R,3S)-3-aminocyclopentanol hydrogen chloride salt, pyridin-3-ylboronic acid and (4-chlorophenyl)boronic acid, the title compound was obtained as described for the example 1 (Scheme 1. General procedure A.).

1H NMR (400 MHz, CDCl3): δ [ppm]=9.46 (s, 1H), 8.71 (d, J=4.1 Hz, 1H), 8.61 (s, 1H), 8.58-8.48 (m, 2H), 8.18 (d, J=8.5 Hz, 2H), 7.53-7.42 (m, 3H), 5.35-5.24 (m, 1H), 4.57-4.48 (m, 1H), 2.60-2.48 (m, 1H), 2.41-2.31 (m, 1H), 2.29-2.17 (m, 1H), 2.10-1.94 (m, 2H), 1.89-1.77 (m, 1H); MS (ESI, m/z): 419.13 [M+H]⁺

### Example 33. 3-((1S,3R)-3-hydroxycyclopentyl)-8-(pyridin-3-yl)-6-(4-(trifluoromethyl)phenyl)pyrido[3,4-d]pyrimidin-4(3H)-one

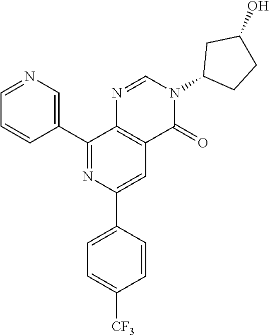 [SMILES: O=c1c2cc(-c3ccc(C(F)(F)F)cc3)nc(-c3cccnc3)c2ncn1[C@H]1CC[C@@H](O)C1]

Using (1R,3S)-3-aminocyclopentanol hydrogen chloride salt, pyridin-3-ylboronic acid and (4-(trifluoromethyl)phenyl)boronic acid, the title compound was obtained as described for the example 1 (Scheme 1. General procedure A.).

1H NMR (400 MHz, CDCl3): δ [ppm]=9.47 (d, J=1.6 Hz, 1H), 8.73 (dd, J=4.8, 1.2 Hz, 1H), 8.63 (s, 1H), 8.62 (s, 1H), 8.60-8.52 (m, 1H), 8.36 (d, J=8.2 Hz, 2H), 7.78 (d, J=8.3 Hz, 2H), 7.48 (dd, J=7.9, 4.9 Hz, 1H), 5.35-5.24 (m, 1H), 4.57-4.48 (m, 1H), 2.67 (d, J=4.4 Hz, 1H), 2.61-2.47 (m, 1H), 2.44-2.32 (m, 1H), 2.30-2.16 (m, 1H), 2.09-1.92 (m, 2H), 1.91-1.77 (m, J=12.6, 6.2 Hz, 1H); MS (ESI, m/z): 453.16 [M+H]⁺

### Example 34. 6-(4-chlorophenyl)-3-((1S,3R)-3-hydroxycyclopentyl)-8-(1-methyl-1H-pyrazol-4-yl)pyrido[3,4-d]pyrimidin-4(3H)-one

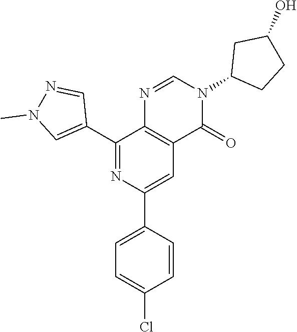 [SMILES: Cn1cc(-c2nc(-c3ccc(Cl)cc3)cc3c(=O)n([C@H]4CC[C@@H](O)C4)cnc23)cn1]

Using (1R,3S)-3-aminocyclopentanol hydrogen chloride salt, (1-methyl-1H-pyrazol-4-yl)boronic acid and (4-chlorophenyl)boronic acid, the title compound was obtained as described for the example 1 (Scheme 1. General procedure A.).

1H NMR (400 MHz, CDCl3): δ [ppm]=8.63 (s, 1H), 8.58 (s, 1H), 8.56 (s, 1H), 8.32 (s, 1H), 8.17 (d, J=8.6 Hz, 2H), 7.49 (d, J=8.7 Hz, 2H), 5.27-5.21 (m, 1H), 4.56-4.51 (m, 1H), 4.02 (s, 3H), 2.78 (d, J=5.0 Hz, 1H), 2.58-2.52 (m, 1H), 2.34-2.19 (m, 2H), 2.07-1.97 (m, 2H), 1.88-1.81 (m, 1H); MS (ESI, m/z): 422.15 [M+H]⁺

### Example 35. 3-((1S,3R)-3-hydroxycyclopentyl)-8-(1-methyl-1H-pyrazol-4-yl)-6-(4-(trifluoromethyl)phenyl)pyrido[3,4-d]pyrimidin-4(3H)-one

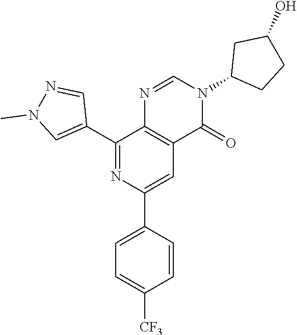 [SMILES: Cn1cc(-c2nc(-c3ccc(C(F)(F)F)cc3)cc3c(=O)n([C@H]4CC[C@@H](O)C4)cnc23)cn1]

Using (1R,3S)-3-aminocyclopentanol hydrogen chloride salt, (1-methyl-1H-pyrazol-4-yl)boronic acid and (4-(trifluoromethyl)phenyl)boronic acid, the title compound was obtained as described for the example 1 (Scheme 1. General procedure A.).

1H NMR (400 MHz, CDCl3): δ [ppm]=8.64 (s, 1H), 8.60 (s, 1H), 8.59 (s, 1H), 8.38 (s, 1H), 8.33 (d, J=8.2 Hz, 2H), 7.77 (d, J=8.3 Hz, 2H), 5.30-5.22 (m, 1H), 4.58-4.49 (m, 1H), 4.03 (s, 3H), 2.77 (d, J=4.8 Hz, 1H), 2.60-2.52 (m, 1H), 2.39-2.31 (m, 1H), 2.30-2.21 (m, 1H), 2.09-1.98 (m, 2H), 1.90-1.80 (m, 1H); MS (ESI, m/z): 456.20 [M+H]⁺

### Example 36. 1-(6-(4-chlorophenyl)-4-oxo-8-(pyridin-3-yl)pyrido[3,4-d]pyrimidin-3(4H)-yl)-2-methylpropan-2-yl acetate

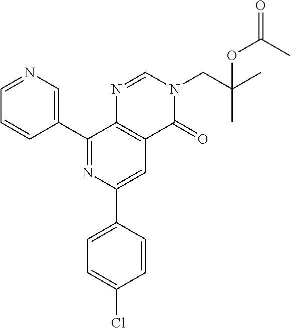 [SMILES: CC(=O)OC(C)(C)Cn1cnc2c(-c3cccnc3)nc(-c3ccc(Cl)cc3)cc2c1=O]

Using 1-amino-2-methylpropan-2-ol, pyridin-3-ylboronic acid and (4-chlorophenyl)boronic acid, the general procedure A (Scheme 1) was followed. In the course of cyclization step c, an additional acetylation on the hydroxyl group was observed based on LC-MS and ¹H NMR analysis. The title compound was obtained as described for the example 1 (Scheme 1. General procedure A.).

1H NMR (400 MHz, CDCl3): δ [ppm]=9.47 (s, 1H), 8.73 (d, J=4.1 Hz, 1H), 8.59-8.51 (m, 2H), 8.19 (d, J=8.6 Hz, 2H), 8.15 (s, 1H), 7.54-7.44 (m, 3H), 4.36 (s, 2H), 2.06 (s, 3H), 1.57 (s, 6H); MS (ESI, m/z): 449.20 [M+H]⁺

### Example 37. 2-methyl-1-(4-oxo-8-(pyridin-3-yl)-6-(4-(trifluoromethyl)phenyl)pyrido[3,4-d]pyrimidin-3(4H)-yl)propan-2-yl acetate

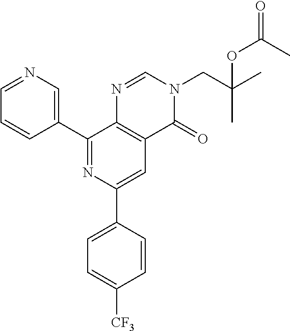 [SMILES: CC(=O)OC(C)(C)Cn1cnc2c(-c3cccnc3)nc(-c3ccc(C(F)(F)F)cc3)cc2c1=O]

Using 1-amino-2-methylpropan-2-ol, pyridin-3-ylboronic acid and (4-(trifluoromethyl)phenyl)boronic acid, the general procedure A (Scheme 1) was followed. In the course of cyclization step c, an additional acetylation on the hydroxyl group was observed based on LC-MS and ¹H NMR analysis. The title compound was obtained as described for the example 1 (Scheme 1. General procedure A.).

1H NMR (400 MHz, CDCl3): δ [ppm]=9.49 (s, 1H), 8.74 (d, J=4.0 Hz, 1H), 8.62 (s, 1H), 8.56 (ddd, J=7.9, 1.8, 1.8 Hz, 1H), 8.35 (d, J=8.2 Hz, 2H), 8.17 (s, 1H), 7.78 (d, J=8.3 Hz, 2H), 7.53-7.46 (m, 1H), 4.37 (s, 2H), 2.06 (s, 3H), 1.59 (s, 6H); MS (ESI, m/z): 483.12 [M+H]⁺

### Example 38. 6-(4-chlorophenyl)-3-(2-hydroxy-2-methylpropyl)-8-(pyridin-3-yl)pyrido[3,4-d]pyrimidin-4(3H)-one

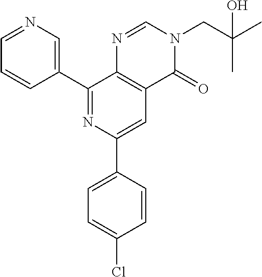 [SMILES: CC(C)(O)Cn1cnc2c(-c3cccnc3)nc(-c3ccc(Cl)cc3)cc2c1=O]

Using 1-amino-2-methylpropan-2-ol, pyridin-3-ylboronic acid and (4-chlorophenyl)boronic acid, the general procedure A (Scheme 1) was followed. In the course of cyclization step c, an additional acetylation on the hydroxyl group was observed based on LC-MS and ¹H NMR analysis. The acetylation intermediate was deprotected after the second Suzuki coupling reaction under the condition of K₂CO₃ in MeOH:Water (5:1) at rt to provide entitled compound of the example 1 (Scheme 1. General procedure A.).

1H NMR (400 MHz, CDCl3): δ [ppm]=9.43 (s, 1H), 8.69 (d, J=3.8 Hz, 1H), 8.55 (d, J=8.0 Hz, 1H), 8.52 (s, 1H), 8.27 (s, 1H), 8.16 (d, J=8.6 Hz, 2H), 7.49 (d, J=8.6 Hz, 2H), 7.46-7.38 (m, 1H), 4.14 (s, 2H), 1.37 (s, 6H); MS (ESI, m/z): 407.24 [M+H]⁺

### Example 39. 3-(2-hydroxy-2-methylpropyl)-8-(pyridin-3-yl)-6-(4-(trifluoromethyl)phenyl)pyrido[3,4-d]pyrimidin-4(3H)-one

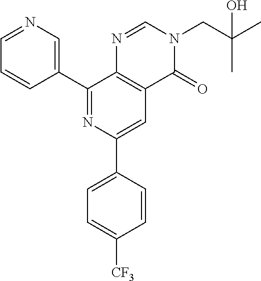 [SMILES: CC(C)(O)Cn1cnc2c(-c3cccnc3)nc(-c3ccc(C(F)(F)F)cc3)cc2c1=O]

Using 1-amino-2-methylpropan-2-ol, pyridin-3-ylboronic acid and (4-(trifluoromethyl)phenyl)boronic acid, the general procedure A (Scheme 1) was followed. In the course of cyclization step c, an additional acetylation on hydroxyl groups was observed based on LC-MS and 1H NMR analysis. The acetylation intermediate was deprotected after the second Suzuki coupling reaction under the condition of K₂CO₃ in MeOH:Water (5:1) at rt to provide entitled compound of the example 1 (Scheme 1. General procedure A.).

1H NMR (400 MHz, CDCl3): δ [ppm]=9.47 (s, 1H), 8.73 (d, J=3.2 Hz, 1H), 8.62 (s, 1H), 8.58 (d, J=8.0 Hz, 1H), 8.35 (d, J=8.2 Hz, 2H), 8.30 (s, 1H), 7.78 (d, J=8.3 Hz, 2H), 7.51-7.44 (m, 1H), 4.15 (s, 2H), 1.37 (s, 6H); MS (ESI, m/z): 441.31 [M+H]⁺

### Example 40. 3-(2-hydroxy-2-methylpropyl)-8-(pyridin-3-yl)-6-(6-(trifluoromethyl)pyridin-3-yl)pyrido[3,4-d]pyrimidin-4(3H)-one

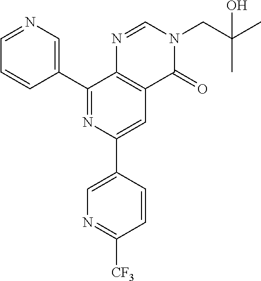 [SMILES: CC(C)(O)Cn1cnc2c(-c3cccnc3)nc(-c3ccc(C(F)(F)F)nc3)cc2c1=O]

Using 1-amino-2-methylpropan-2-ol, pyridin-3-ylboronic acid and (6-(trifluoromethyl)pyridin-3-yl)boronic acid, the general procedure A (Scheme 1) was followed. In the course of cyclization step c, an additional acetylation on hydroxyl groups was observed by LC-MS analysis. The acetylation intermediate was deprotected after the second Suzuki coupling reaction under the condition of K₂CO₃ in MeOH:Water (5:1) at rt to provide entitled compound of the example 1 (Scheme 1. General procedure A.).

1H NMR (400 MHz, CDCl3): δ [ppm]=9.52 (d, J=1.9 Hz, 1H), 9.48 (d, J=1.8 Hz, 1H), 8.76-8.67 (m, 2H), 8.65 (s, 1H), 8.60-8.55 (m, 1H), 8.35 (s, 1H), 7.84 (d, J=8.2 Hz, 1H), 7.48 (dd, J=8.0, 4.8 Hz, 1H), 4.16 (s, 2H), 1.37 (s, 6H); MS (ESI, m/z): 442.17 [M+H]⁺

### Example 41. 6-(4-chlorophenyl)-3-(1-hydroxy-3-methylbutan-2-yl)-8-(pyridin-3-yl)pyrido[3,4-d]pyrimidin-4(3H)-one

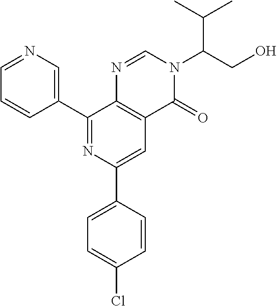 [SMILES: CC(C)C(CO)n1cnc2c(-c3cccnc3)nc(-c3ccc(Cl)cc3)cc2c1=O]

Using 2-amino-3-methylbutan-1-ol, pyridin-3-ylboronic acid and (4chlorophenyl)boronic acid, the title compound was obtained as described for the example 1 (Scheme 1. General procedure A.).

1H-NMR (400 MHz, CDCl₃): δ [ppm]=9.43 (d, J=1.8 Hz, 1H), 8.68 (dd, J=4.8, 1.5 Hz, 1H), 8.54 (dt, J=7.8, 1.9 Hz, 1H), 8.50 (s, 1H), 8.30 (s, 1H), 8.15 (d, J=8.6 Hz, 2H), 7.48 (d, J=8.6 Hz, 2H), 7.45 (dd, J=8.2, 5.2 Hz, 1H), 4.51 (br, 1H), 4.25-4.16 (m, 1H), 4.01 (dd, J=11.8, 2.2 Hz, 1H), 2.57-2.43 (m, 2H), 1.20 (d, J=6.5 Hz, 3H), 0.89 (d, J=6.6 Hz, 3H); MS (ESI, m/z): 421.20 [M+H]⁺

### Example 42. 3-(1-hydroxy-3-methylbutan-2-yl)-8-(pyridin-3-yl)-6-(4-(trifluoromethyl)phenyl)pyrido[3,4-d]pyrimidin-4(3H)-one

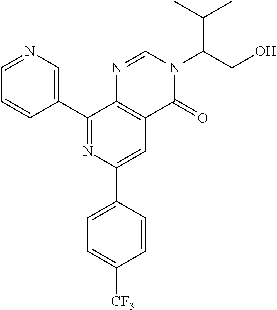 [SMILES: CC(C)C(CO)n1cnc2c(-c3cccnc3)nc(-c3ccc(C(F)(F)F)cc3)cc2c1=O]

Using 2-amino-3-methylbutan-1-ol, pyridin-3-ylboronic acid and (4-(trifluoromethyl)phenyl)boronic acid, the title compound was obtained as described for the example 1 (Scheme 1. General procedure A.).

1H-NMR (400 MHz, CDCl₃): δ [ppm]=9.42 (s, 1H), 8.67 (dd, J=4.8, 1.4 Hz, 1H), 8.55-8.52 (m, 1H), 8.54 (s, 1H), 8.33 (s, 1H), 8.29 (d, J=8.2 Hz, 2H), 7.75 (d, J=8.3 Hz, 2H), 7.44 (dd, J=7.7, 5.1 Hz, 1H), 4.52 (br, 1H), 4.26-4.18 (m, 1H), 4.02 (dd, J=11.9, 2.2 Hz, 1H), 2.91-2.74 (m, 1H), 2.55-2.44 (m, 1H), 1.20 (d, J=6.5 Hz, 3H), 0.89 (d, J=6.7 Hz, 3H); MS (ESI, m/z): 455.21 [M+H]⁺

### Example 43. 3-(1-hydroxy-3-methylbutan-2-yl)-8-(pyridin-3-yl)-6-(6-(trifluoromethyl)pyridin-3-yl)pyrido[3,4-d]pyrimidin-4(3H)-one

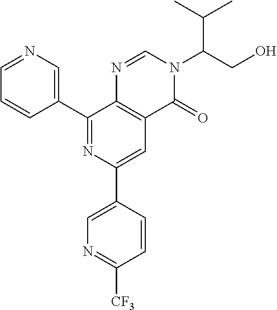 [SMILES: CC(C)C(CO)n1cnc2c(-c3cccnc3)nc(-c3ccc(C(F)(F)F)nc3)cc2c1=O]

Using 2-amino-3-methylbutan-1-ol, pyridin-3-ylboronic acid and (6-(trifluoromethyl)pyridin-3-yl)boronic acid, the title compound was obtained as described for the example 1 (Scheme 1. General procedure A.).

1H-NMR (400 MHz, CDCl₃): δ [ppm]=9.50 (d, J=1.5 Hz, 1H), 9.46 (d, J=1.6 Hz, 1H), 8.72 (dd, J=4.8, 1.4 Hz, 1H), 8.68 (dd, J=8.3, 1.7 Hz, 1H), 8.62 (s, 1H), 8.56 (dt, J=8.0, 1.9 Hz, 1H), 8.37 (s, 1H), 7.83 (d, J=8.2 Hz, 1H), 7.47 (dd, J=7.9, 4.9 Hz, 1H), 4.55 (br, 1H), 4.26-4.17 (m, 1H), 4.02 (dt, J=11.7, 3.1 Hz, 1H), 2.55-2.43 (m, 1H), 2.34-2.24 (m, 1H), 1.21 (d, J=6.5 Hz, 3H), 0. 90 (d, J=6.7 Hz, 3H); MS (ESI, m/z): 456.19 [M+H]⁺

### Example 44. (S)-2-((6-(4-chlorophenyl)-2-(pyridin-3-yl)pyrimidin-4-yl)amino)propan-1-ol

Scheme for the preparation of the Compound of Example 44:

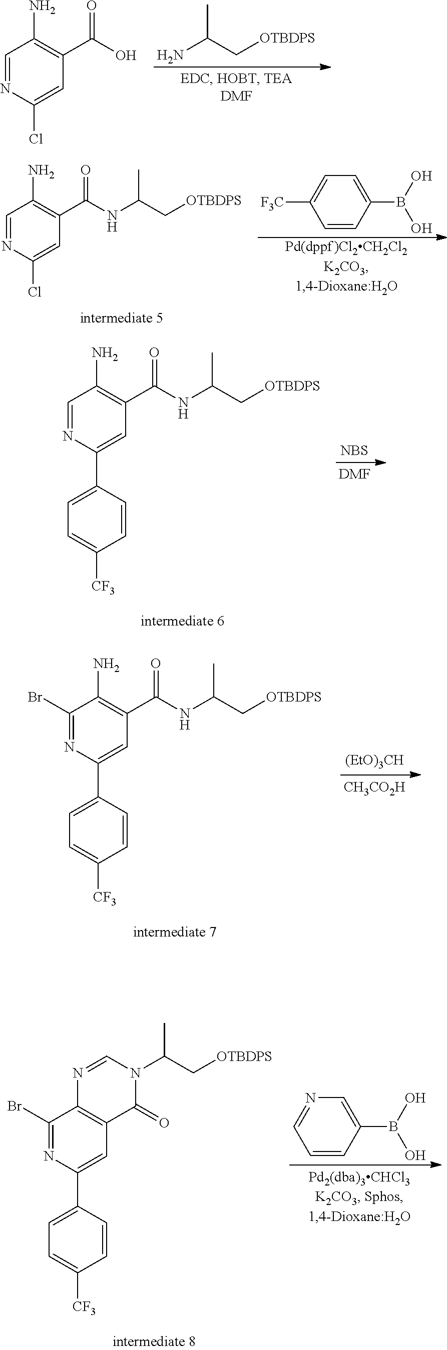

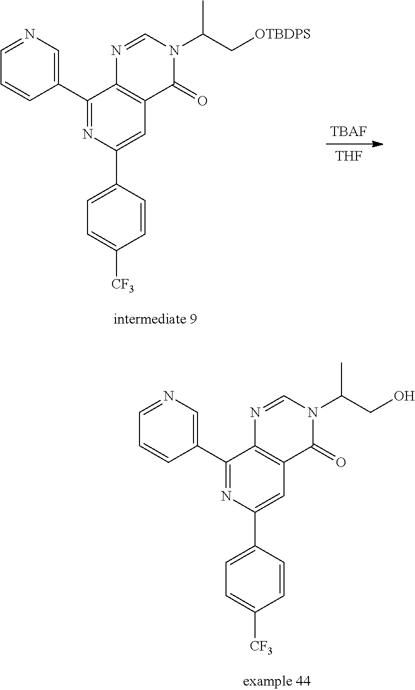

### Intermediate 5. 5-amino-N-(1-((tert-butyldiphenylsilyl)oxy)propan-2-yl)-2-chloroisonicotinamide

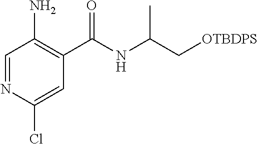

5-Amino-2-chloroisonicotinic acid (1 g, 5.79 mmol, 1 equiv.), EDC (1.33 g, 6.95 mmol, 1.2 equiv.), HOBT (1.06 g, 6.95 mmol, 1.2 equiv.) and TEA (1.21 mL, 8.69 mmol, 1.5 equiv.) were dissolved in DMF (10 mL, 0.6 M) and stirred for 5 min. Then, 1-((tert-butyldiphenylsilyl)oxy)propan-2-amine (2.73 g, 8.69 mmol, 1.5 equiv.) was added to the reaction mixture and stirred for 12h at 60° C. The reaction mixture was diluted with water (50 mL), extracted with EtOAc (20 mL×3), washed with brine (20 mL×3), dried over Na₂SO₄ and glass filtered. The filtrate was evaporated in vacuo and purified by MPLC (silica gel, 0-30% EtOAc/Hexane) to give 0.87 g (32% yield) of 5-amino-N-(1-((tert-butyldiphenylsilyl)oxy)propan-2-yl)-2-chloroisonicotinamide (intermediate 5).

MS (ESI, m/z):468.04 [M+H]⁺

### Intermediate 6. 5-amino-N-(1-((tert-butyldiphenylsilyl)oxy)propan-2-yl)-2-(4-(trifluoro methyl)phenyl)isonicotinamide

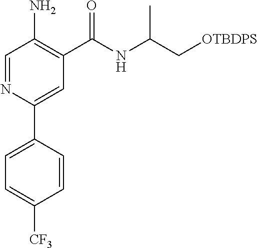

5-Amino-N-(1-((tert-butyldiphenylsilyl)oxy)propan-2-yl)-2-chloroisonicotinamide (intermediate 5) (0.64 g, 1.37 mmol, 1 equiv.), (4-(trifluoromethyl)phenyl)boronic acid (0.52 g, 2.73 mmol, 2 equiv.), K₂CO₃ (0.57 g, 4.10 mmol, 3 equiv.) and Pd(dppf)Cl₂.CH₂Cl₂(0.11 g, 0.14 mmol, 0.1 equiv.) were dissolved in 1,4-Dioxane/Water (4 mL/1 mL, 0.4 M). The reaction mixture was stirred and heated in a Biotage microwave initiator at 130° C. for 1 h. The reaction mixture was concentrated under reduced pressure and directly subjected to purification by MPLC (silica gel, 0˜30% EtOAc/Hexane) to give 0.48 g (61% yield) of 5-amino-N-(1-((tert-butyldiphenylsilyl)oxy)propan-2-yl)-2-(4-(trifluoromethyl) phenyl)isonicotinamide (intermediate 6).

MS (ESI, m/z): 578.16 [M+H]⁺

### Intermediate 7. 3-amino-2-bromo-N-(1-((tert-butyldiphenylsilyl)oxy)propan-2-yl)-6-(4-(trifluoromethyl)phenyl)isonicotinamide

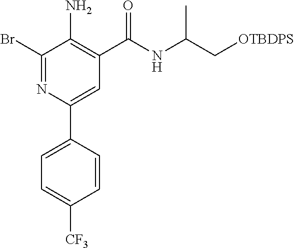

5-amino-N-(1-((tert-butyldiphenylsilyl)oxy)propan-2-yl)-2-(4(trifluoromethyl)phenyl) isonicotinamide (intermediate 6) (0.1 g, 0.17 mmol, 1 equiv.) and N-bromosuccinimide (0.046 g, 0.26 mmol, 1.5 equiv.) were dissolved in DMF (5 mL, 0.035 M) and stirred for 1 h at RT. The reaction mixture was diluted with water (20 mL), extracted with EtOAc (20 mL×3), washed with brine (10 mL×3), dried over Na₂SO₄ and glass filtered. The filtrate was evaporated in vacuo to give 0.082 g (72% yield) of 3-amino-2-bromo-N-(1-((tert-butyldiphenylsilyl)oxy)propan-2-yl)-6-(4-(trifluoromethyl)phenyl)isonicotinamide (intermediate 7).

MS (ESI, m/z):656.14 [M+H]⁺

### Intermediate 8. 8-bromo-3-(1-((tert-butyldiphenylsilyl)oxy)propan-2-yl)-6-(4-(trifluoro methyl)phenyl)pyrido[3,4-d]pyrimidin-4(3H)-one

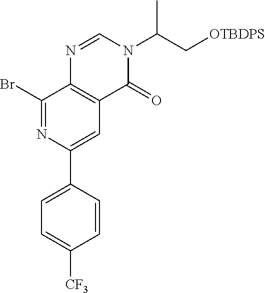

3-Amino-2-bromo-N-(1-((tert-butyldiphenylsilyl)oxy)propan-2-yl)-6-(4-(trifluoromethyl)phenyl) isonicotinamide (intermediate 7) (82 mg, 0.125 mmol, 1 equiv.) was dissolved in a solution of (EtO)₃CH (1.5 mL, 9.01 mmol, 9 equiv.) and CH₃CO₂H (1.5 mL, 26.2 mmol, 210 equiv.). The reaction mixture was stirred and heated in a Biotage microwave initiator at 150° C. for 2h. The reaction mixture was diluted with water (20 mL), extracted with EtOAc (20 mL×3), washed with brine (10 mL×3), dried over Na₂SO₄ and glass filtered. The filtrate was evaporated in vacuo and used without further purification to give 0.06 g (72% yield) of 8-bromo-3-(1-((tert-butyldiphenylsilyl)oxy)propan-2-yl)-6-(4-(trifluoromethyl)phenyl)pyrido[3,4-d]pyrimidin-4(3H)-one (intermediate 8).

MS (ESI, m/z):666.09 [M]⁺

### Intermediate 9. 3-(1-((tert-butyldiphenylsilyl)oxy)propan-2-yl)-8-(pyridin-3-yl)-6-(4-(trifluoromethyl)phenyl)pyrido[3,4-d]pyrimidin-4(3H)-one

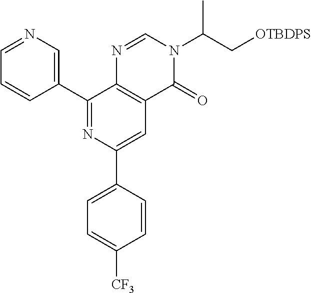

8-bromo-3-(1-((tert-butyldiphenylsilyl)oxy)propan-2-yl)-6-(4-(trifluoromethyl)phenyl)pyrido[3,4-d]pyrimidin-4(3H)-one (intermediate 8) (60 mg, 0.090 mmol, 1 equiv.), pyridin-3-ylboronic acid (17 mg, 0.13 mmol, 1.5 equiv.), K₂CO₃ (37 mg, 0.27 mmol, 3 equiv.), Sphos (3.7 mg, 9.00 μmol, 0.1 equiv.) and Pd₂(dba)₃.CHCl₃ (9.32 mg, 9.00 μmol, 0.1 equiv.) were dissolved in 1,4-Dioxane/Water (4 mL/1 mL, 0.2 M). The reaction mixture was stirred and heated in a Biotage microwave initiator at 130° C. for 1 h. The reaction mixture was concentrated under reduced pressure and directly subjected to purification by MPLC (silica gel, 0˜30% EtOAc/Hexane) to give 10 mg (17% yield) of 3-(1-((tert-butyldiphenylsilyl)oxy)propan-2-yl)-8-(pyridin-3-yl)-6-(4-(trifluoromethyl)phenyl)pyrido[3,4-d]pyrimidin-4(3H)-one (intermediate 9).

MS (ESI, m/z):665.15[M+H]⁺

### Example 44. 3-(1-hydroxypropan-2-yl)-8-(pyridin-3-yl)-6-(4-(trifluoromethyl)phenyl)pyrido[3,4-d]pyrimidin-4(3H)-one

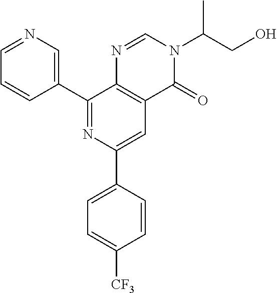 [SMILES: CC(CO)n1cnc2c(-c3cccnc3)nc(-c3ccc(C(F)(F)F)cc3)cc2c1=O]

A solution of 3-(1-((tert-butyldiphenylsilyl)oxy)propan-2-yl)-8-(pyridin-3-yl)-6-(4-(trifluoro methyl) phenyl)pyrido[3,4-d]pyrimidin-4(3H)-one (intermediate 9) (10 mg, 0.015 mmol, 1 equiv.) and TBAF (1.0 M in THF, 15 μL, 0.015 mmol, 1 equiv.) in THF (2 mL, 0.0075 M) was stirred for 12h at 40° C. The reaction mixture was diluted with water (5 mL), extracted with EtOAc (5 mL×3), washed with brine (5 mL×3), dried over Na₂SO₄ and glass filtered. The filtrate was evaporated in vacuo and purified by Prep TLC (silica gel, 0-50% EtOAc/Hexane) to give 1.9 mg (30% yield) of 3-(1-hydroxypropan-2-yl)-8-(pyridin-3-yl)-6-(4-(trifluoromethyl) phenyl)pyrido[3,4-d]pyrimidin-4(3H)-one (example 44).

1H-NMR (400 MHz, CD₃OD): δ [ppm]=9.44 (d, J=1.6 Hz, 1H), 8.75 (d, J=8.0 Hz, 1H), 8.69 (s, 1H), 8.66 (d, J=4.8 Hz, 1H), 8.48 (s, 1H), 8.45 (d, J=8.2 Hz, 2H), 7.86 (d, J=8.3 Hz, 2H), 7.63 (dd, J=8.0, 5.0 Hz, 1H), 5.08-4.99 (m, 1H), 3.99 (dd, J=11.9, 7.0 Hz, 1H), 3.88 (dd, J=11.9, 4.3 Hz, 1H), 1.58 (d, J=7.1 Hz, 3H); MS (ESI, m/z): 426.94 [M+H]⁺

### Example 45. 3-(1-hydroxypropan-2-yl)-8-(1-methyl-1H-pyrazol-4-yl)-6-(4-(trifluoromethyl)phenyl)pyrido[3,4-d]pyrimidin-4(3H)-one

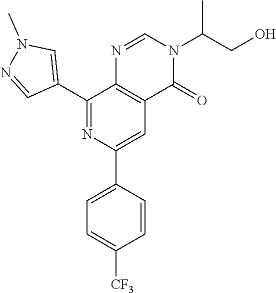 [SMILES: CC(CO)n1cnc2c(-c3cnn(C)c3)nc(-c3ccc(C(F)(F)F)cc3)cc2c1=O]

Using (1-methyl-1H-pyrazol-4-yl)boronic acid, the title compound was obtained as described for the example 44 (Scheme 2. General procedure B.).

1H-NMR (400 MHz, CD₃OD): δ [ppm]=8.69 (s, 1H), 8.46 (s, 1H), 8.38 (s, 1H), 8.31 (s, 1H), 8.29 (d, J=2.7 Hz, 2H), 7.71 (d, J=8.3 Hz, 2H), 4.94-4.86 (m, 1H), 3.90 (s, 3H), 3.87 (t, J=5.9 Hz, 1H), 3.77 (dd, J=11.9, 4.3 Hz, 1H), 1.47 (d, J=7.1 Hz, 3H); MS (ESI, m/z): 430.02 [M+H]⁺

### Example 46. 6-(4-chlorophenyl)-3-(1-hydroxypropan-2-yl)-8-(pyridin-3-yl)pyrido[3,4-d]pyrimidin-4(3H)-one

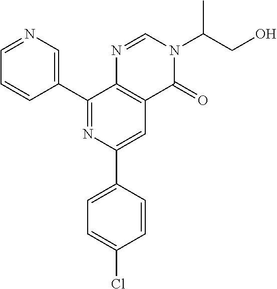 [SMILES: CC(CO)n1cnc2c(-c3cccnc3)nc(-c3ccc(Cl)cc3)cc2c1=O]

Using pyridin-3-ylboronic acid, the title compound was obtained as described for the example 44 (Scheme 2. General procedure B.).

1H-NMR (400 MHz, CD₃OD): δ [ppm]=9.31 (s, 1H), 8.65-8.59 (m, 1H), 8.53 (d, J=4.2 Hz, 1H), 8.49 (s, 1H), 8.34 (s, 1H), 8.14 (d, J=8.6 Hz, 2H), 7.51 (dd, J=7.9, 5.0 Hz, 1H), 7.44 (d, J=8.6 Hz, 2H), 4.95-4.86 (m, 1H), 3.87 (dd, J=11.9, 7.0 Hz, 1H), 3.76 (dd, J=11.9, 4.3 Hz, 1H), 1.46 (d, J=7.1 Hz, 3H); MS (ESI, m/z): 392.91 [M+H]⁺

### Example 47. 2-(6-(4-chlorophenyl)-4-oxo-8-(pyridin-3-yl)pyrido[3,4-d]pyrimidin-3(4H)-yl)propyl diethyl phosphate

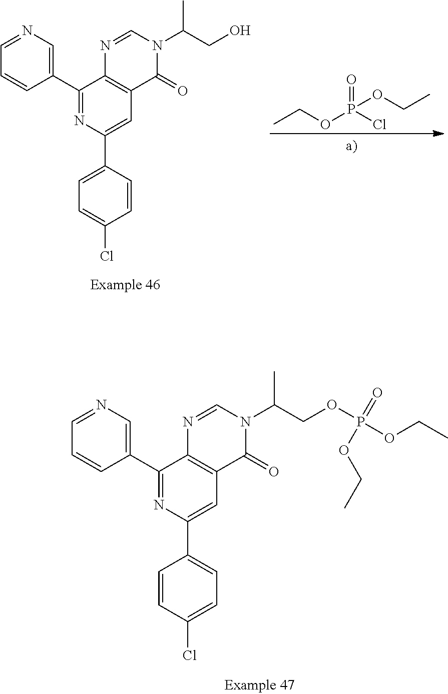

6-(4-chlorophenyl)-3-(1-hydroxypropan-2-yl)-8-(pyridin-3-yl)pyrido[3,4-d]pyrimidin-4(3H)-one (Example 46) (100 mg, 0.255 mmol, 1 equiv.) and sodium hydride (18.33 mg, 0.764 mmol, 3 equiv.) were dissolved in THF (1273 μl, 0.2 M) and stirred for 10 min. Then, a solution of diethyl chlorophosphate (73.8 μl, 0.509 mmol, 2 equiv.) and N,N-dimethylpyridin-4-amine (15.55 mg, 0.127 mmol, 0.5 equiv.) in THF (1273 μl, 0.2 M) was added dropwise to the reaction mixture and stirred for 12h at rt. The reaction mixture was quenched with water (10 mL), extracted with DCM (10 mL×3), washed with brine (10 mL×3), dried over Na₂SO₄ and glass filtered. The filtrate was evaporated in vacuo and used without further purification to give 79 mg (59% yield) of 2-(6-(4-chlorophenyl)-4-oxo-8-(pyridin-3-yl)pyrido[3,4-d]pyrimidin-3(4H)-yl)propyl diethyl phosphate (example 47).

1H-NMR (400 MHz, CDCl₃): δ [ppm]=9.45 (d, J=1.6 Hz, 1H), 8.72 (dd, J=4.8, 1.4 Hz, 1H), 8.55 (dt, J=8.0, 1.9 Hz, 1H), 8.53 (s, 1H), 8.18 (d, J=8.5 Hz, 3H), 7.49 (d, J=8.7 Hz, 2H), 7.47 (dd, J=5.2, 3.1, 1H), 5.15 (dd, J=10.2, 6.3 Hz, 1H), 4.45 (ddd, J=11.2, 7.5, 6.2 Hz, 1H), 4.37-4.28 (m, 1H), 4.06 (p, J=7.3 Hz, 4H), 1.65 (d, J=7.2 Hz, 3H), 1.29-1.23 (m, 6H); MS (ESI, m/z): 529.23 [M+H]⁺

### Example 48. 6-(4-chlorophenyl)-3-(1-hydroxypropan-2-yl)-8-(1-methyl-1H-pyrazol-4-yl)pyrido[3,4-d]pyrimidin-4(3H)-one

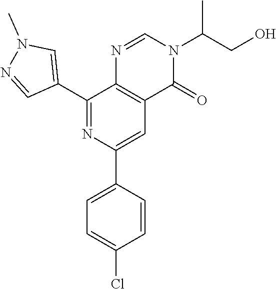 [SMILES: CC(CO)n1cnc2c(-c3cnn(C)c3)nc(-c3ccc(Cl)cc3)cc2c1=O]

Using (1-methyl-1H-pyrazol-4-yl)boronic acid and (4-chlorophenyl)boronic acid, the title compound was obtained as described for the example 44 (Scheme 2. General procedure B.).

1H-NMR (400 MHz, CD₃OD): δ [ppm]=8.79 (s, 1H), 8.56 (s, 1H), 8.46 (s, 1H), 8.33 (s, 1H), 8.22 (d, J=8.4 Hz, 2H), 7.77 (s, 1H), 7.52 (d, J=8.4 Hz, 2H), 5.03-4.99 (m, 1H), 4.01 (s, 3H), 3.99-3.93 (m, 1H), 3.86 (dd, J=11.8, 4.3 Hz, 1H), 1.57 (d, J=7.1 Hz, 3H); MS (ESI, m/z): 396.00 [M+H]⁺

### Example 49. 3-(1-hydroxypropan-2-yl)-8-(pyridin-3-yl)-6-(4-(trifluoromethoxy)phenyl)pyrido[3,4-d]pyrimidin-4(3H)-one

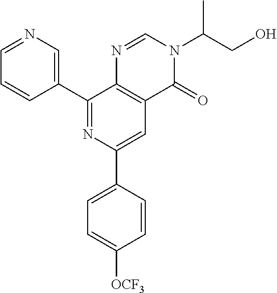 [SMILES: CC(CO)n1cnc2c(-c3cccnc3)nc(-c3ccc(OC(F)(F)F)cc3)cc2c1=O]

Using pyridin-3-ylboronic acid and (4-(trifluoromethoxy)phenyl)boronic acid, the title compound was obtained as described for the example 44 (Scheme 2. General procedure B.).

1H-NMR (400 MHz, CD₃OD): δ [ppm]=9.43 (s, 1H), 8.74 (dd, J=6.2, 1.8 Hz, 1H), 8.68-8.64 (m, 1H), 8.62 (s, 1H), 8.46 (s, 1H), 8.37 (d, J=8.8 Hz, 2H), 7.63 (dd, J=7.9, 5.0 Hz, 1H), 7.46 (d, J=8.4 Hz, 2H), 5.08-4.98 (m, 1H), 3.99 (dd, J=11.9, 7.0 Hz, 1H), 3.88 (dd, J=11.9, 4.3 Hz, 1H), 1.58 (d, J=7.1 Hz, 3H); MS (ESI, m/z): 442.93 [M+H]⁺

### Example 50. 3-(1-hydroxypropan-2-yl)-8-(pyridin-3-yl)-6-(6-(trifluoromethyl)pyridin-3-yl)pyrido[3,4-d]pyrimidin-4(3H)-one

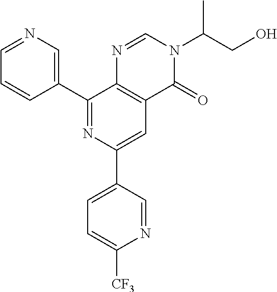 [SMILES: CC(CO)n1cnc2c(-c3cccnc3)nc(-c3ccc(C(F)(F)F)nc3)cc2c1=O]

Using pyridin-3-ylboronic acid and (6-(trifluoromethyl)pyridin-3-yl)boronic acid, the title compound was obtained as described for the example 44 (Scheme 2. General procedure B.).

1H-NMR (400 MHz, CD₃OD): δ [ppm]=9.59 (s, 1H), 9.45 (d, J=1.9 Hz, 1H), 8.89 (d, J=8.2 Hz, 1H), 8.79 (s, 1H), 8.76 (d, J=8.0 Hz, 1H), 8.70-8.59 (m, 1H), 8.52 (s, 1H), 8.01 (d, J=8.3 Hz, 1H), 7.65 (dd, J=8.0, 4.9 Hz, 1H), 5.07-5.00 (m, 1H), 4.00 (dd, J=11.9, 7.0 Hz, 1H), 3.88 (dd, J=11.9, 4.3 Hz, 1H), 1.59 (d, J=7.1 Hz, 3H); MS (ESI, m/z): 427.96 [M+H]⁺

### Example 51. 6-(4-chlorophenyl)-3-(1-hydroxybutan-2-yl)-8-(pyridin-3-yl)pyrido[3,4-d]pyrimidin-4(3H)-one

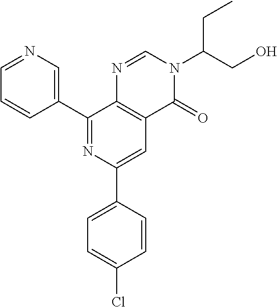 [SMILES: CCC(CO)n1cnc2c(-c3cccnc3)nc(-c3ccc(Cl)cc3)cc2c1=O]

Using 2-aminobutan-1-ol, pyridin-3-ylboronic acid and (4-chlorophenyl)boronic acid, the title compound was obtained as described for the example 44 (Scheme 2. General procedure B.).

1H-NMR (400 MHz, CD₃OD): δ [ppm]=9.43 (d, J=1.6 Hz, 1H), 8.74 (d, J=8.0 Hz, 1H), 8.65 (d, J=4.8 Hz, 1H), 8.61 (s, 1H), 8.44 (s, 1H), 8.26 (d, J=8.5 Hz, 2H), 7.63 (dd, J=8.0, 5.0 Hz, 1H), 7.56 (d, J=8.5 Hz, 2H), 4.84 (s, 1H), 4.04 (dd, J=12.0, 7.1 Hz, 1H), 3.89 (dd, J=12.0, 4.0 Hz, 1H), 2.02 (p, J=7.4 Hz, 2H), 1.00 (t, J=7.4 Hz, 3H); MS (ESI, m/z): 406.95 [M+H]⁺

### Example 52. 6-(4-chlorophenyl)-3-(1-hydroxybutan-2-yl)-8-(1-methyl-1H-pyrazol-4-yl)pyrido[3,4-d]pyrimidin-4(3H)-one

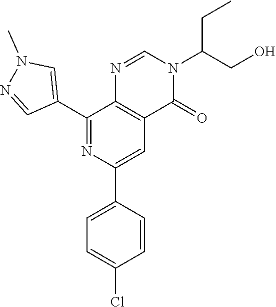 [SMILES: CCC(CO)n1cnc2c(-c3cnn(C)c3)nc(-c3ccc(Cl)cc3)cc2c1=O]

Using 2-aminobutan-1-ol, (1-methyl-1H-pyrazol-4-yl)boronic acid and (4-chlorophenyl)boronic acid, the title compound was obtained as described for the example 44 (Scheme 2. General procedure B.).

1H-NMR (400 MHz, CD₃OD): δ [ppm]=8.79 (s, 1H), 8.55 (s, 1H), 8.45 (s, 1H), 8.31 (s, 1H), 8.21 (d, J=8.5 Hz, 2H), 7.53 (d, J=8.5 Hz, 2H), 4.82 (s, 1H), 4.08-4.03 (m, 1H), 4.02 (s, 3H), 3.89 (dd, J=12.0, 4.0 Hz, 1H), 2.03 (p, J=7.4 Hz, 2H), 1.00 (t, J=7.4 Hz, 3H); MS (ESI, m/z): 409.99 [M+H]⁺

### Example 53. 3-(1-hydroxybutan-2-yl)-8-(pyridin-3-yl)-6-(4-(trifluoromethyl)phenyl)pyrido[3,4-d]pyrimidin-4(3H)-one

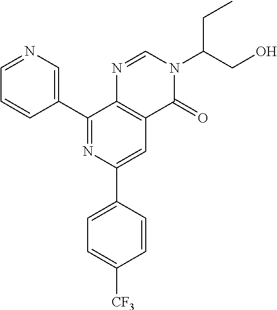 [SMILES: CCC(CO)n1cnc2c(-c3cccnc3)nc(-c3ccc(C(F)(F)F)cc3)cc2c1=O]

Using 2-aminobutan-1-ol, pyridin-3-ylboronic acid and (4-(trifluoromethyl)phenyl)boronic acid, the title compound was obtained as described for the example 44 (Scheme 2. General procedure B.).

1H-NMR (400 MHz, CD₃OD): δ [ppm]=9.43 (d, J=2.0 Hz, 1H), 8.74 (dt, J=8.0, 1.8 Hz, 1H), 8.66 (s, 1H), 8.66-8.63 (m, 1H), 8.46 (s, 1H), 8.43 (d, J=8.3 Hz, 2H), 7.84 (d, J=8.4 Hz, 2H), 7.62 (dd, J=8.0, 5.0 Hz, 1H), 4.84 (s, 1H), 4.04 (dd, J=12.0, 7.0 Hz, 1H), 3.89 (dd, J=12.0, 4.0 Hz, 1H), 2.07-1.94 (m, 2H), 1.01 (t, J=7.4 Hz, 3H); MS (ESI, m/z): 441.00 [M+H]⁺

### Example 54. 3-(1-hydroxybutan-2-yl)-8-(1-methyl-1H-pyrazol-4-yl)-6-(4-(trifluoromethyl)phenyl)pyrido[3,4-d]pyrimidin-4(3H)-one

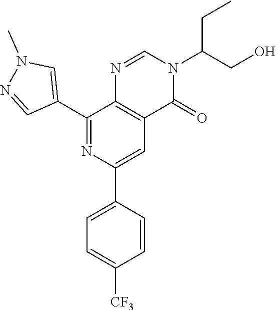 [SMILES: CCC(CO)n1cnc2c(-c3cnn(C)c3)nc(-c3ccc(C(F)(F)F)cc3)cc2c1=O]

Using 2-aminobutan-1-ol, (1-methyl-1H-pyrazol-4-yl)boronic acid and (4-(trifluoromethyl)phenyl)boronic acid, the title compound was obtained as described for the example 44 (Scheme 2. General procedure B.).

1H-NMR (400 MHz, CD₃OD): δ [ppm]=8.78 (s, 1H), 8.55 (s, 1H), 8.47 (s, 1H), 8.41 (s, 1H), 8.38 (s, 2H), 7.82 (d, J=8.1 Hz, 2H), 4.83 (s, 1H), 4.08-4.03 (m, 1H), 4.02 (s, 3H), 3.90 (dd, J=12.0, 3.9 Hz, 1H), 2.03 (t, J=7.5 Hz, 2H), 1.01 (t, J=7.4 Hz, 3H); MS (ESI, m/z): 444.01 [M+H]⁺

### Example 55. 6-(4-chlorophenyl)-8-(3-fluorophenyl)-3-(1-hydroxybutan-2-yl)pyrido[3,4-d]pyrimidin-4(3H)-one

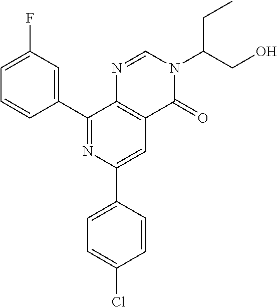 [SMILES: CCC(CO)n1cnc2c(-c3cccc(F)c3)nc(-c3ccc(Cl)cc3)cc2c1=O]

Using 2-aminobutan-1-ol, (3-fluorophenyl)boronic acid and (4-chlorophenyl)boronic acid, the title compound was obtained as described for the example 44 (Scheme 2. General procedure B.).

1H-NMR (400 MHz, CD₃OD): δ [ppm]=8.56 (s, 1H), 8.41 (s, 1H), 8.24 (d, J=8.6 Hz, 2H), 8.12-7.96 (m, 2H), 7.58-7.52 (m, 3H), 7.24 (td, J=8.5, 2.6 Hz, 1H), 4.83 (s, 1H), 4.03 (dd, J=12.0, 7.0 Hz, 1H), 3.88 (dd, J=12.0, 4.1 Hz, 1H), 2.07-1.95 (m, 2H), 1.00 (t, J=7.4 Hz, 3H); MS (ESI, m/z): 423.98 [M+H]⁺

### Example 56. 6-(4-chlorophenyl)-3-((1r,4r)-4-hydroxycyclohexyl)-8-(pyridin-3-yl)pyrido[3,4-d]pyrimidin-4(3H)-one

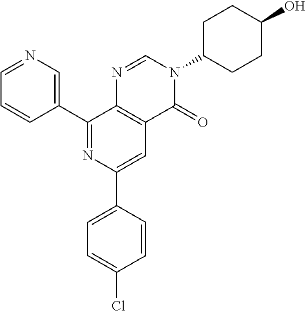

Using (1r,4r)-4-aminocyclohexan-1-ol, pyridin-3-ylboronic acid and (4-chlorophenyl)boronic acid, the title compound was obtained as described for the example 44 (Scheme 2. General procedure B.).

1H-NMR (400 MHz, CDCl₃): δ [ppm]=9.45 (s, 1H), 8.72 (s, 1H), 8.58-8.46 (m, 2H), 8.19 (d, J=8.6 Hz, 2H), 8.17 (s, 1H), 7.54-7.42 (m, 3H), 4.89-4.76 (m, 9H), 3.83-3.74 (m, 1H), 2.32-2.16 (m, 2H), 2.14-2.03 (m, 2H), 1.92-1.76 (m, 2H), 1.69-1.59 (m, 2H); MS (ESI, m/z): 433.01 [M+H]⁺

### Example 57. 3-((1r,4r)-4-hydroxycyclohexyl)-8-(pyridin-3-yl)-6-(4-(trifluoromethyl)phenyl)pyrido[3,4-d]pyrimidin-4(3H)-one

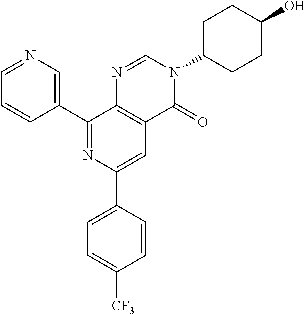

Using (1r,4r)-4-aminocyclohexan-1-ol, pyridin-3-ylboronic acid and (4-(trifluoromethyl)phenyl)boronic acid, the title compound was obtained as described for the example 44 (Scheme 2. General procedure B.).

1H-NMR (400 MHz, CDCl₃): δ [ppm]=9.46 (d, J=1.9 Hz, 1H), 8.73 (dd, J=4.8, 1.6 Hz, 1H), 8.62 (s, 1H), 8.58-8.51 (m, 1H), 8.36 (d, J=8.3 Hz, 2H), 8.20 (s, 1H), 7.79 (d, J=8.4 Hz, 2H), 7.48 (dd, J=7.9, 4.9 Hz, 1H), 4.87-4.79 (m, 1H), 3.83-3.75 (m, 1H), 2.28-2.16 (m, 2H), 2.14-2.03 (m, 2H), 1.92-1.79 (m, 2H), 1.69-1.60 (m, 2H); MS (ESI, m/z): 466.97 [M+H]⁺

### Example 58. 6-(4-chlorophenyl)-3-((1s,4s)-4-hydroxycyclohexyl)-8-(pyridin-3-yl)pyrido[3,4-d]pyrimidin-4(3H)-one

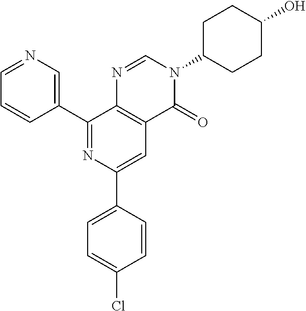

Using methyl (1r,4r)-4-aminocyclohexanol, pyridin-3-ylboronic acid and (4-chlorophenyl)boronic acid, the general procedure B (Scheme 2) was followed. In the course of cyclization step c, an additional acetylation on hydroxyl groups was observed based on LC-MS analysis. The acetylation intermediate was deprotected after the second Suzuki coupling reaction under the condition of K₂CO₃ in MeOH:Water (5:1) at rt to provide entitled compound of the example 58 (Scheme 2. General procedure B.).

¹H NMR (400 MHz, CDCl₃): δ [ppm]=9.46 (s, 1H), 8.72 (s, 1H), 8.56 (d, J=10.0 Hz, 2H), 8.29 (s, 1H), 8.22-8.10 (m, 2H), 7.57-7.36 (m, 3H), 4.90 (tt, J=12.7, 3.5 Hz, 1H), 4.24-4.17 (m, 1H), 2.28-2.11 (m, 2H), 2.09-1.97 (m, 2H), 1.90-1.72 (m, 4H); MS (ESI, m/z): 433.17 [M+H]⁺

### Example 59. 3-(1-hydroxypropan-2-yl)-8-(1-methyl-1H-pyrazol-4-yl)-6-(4-(trifluoromethyl)phenyl)-2,3-dihydropyrido[3,4-d]pyrimidin-4(1H)-one

Scheme for the preparation of the Compound of Example 59:

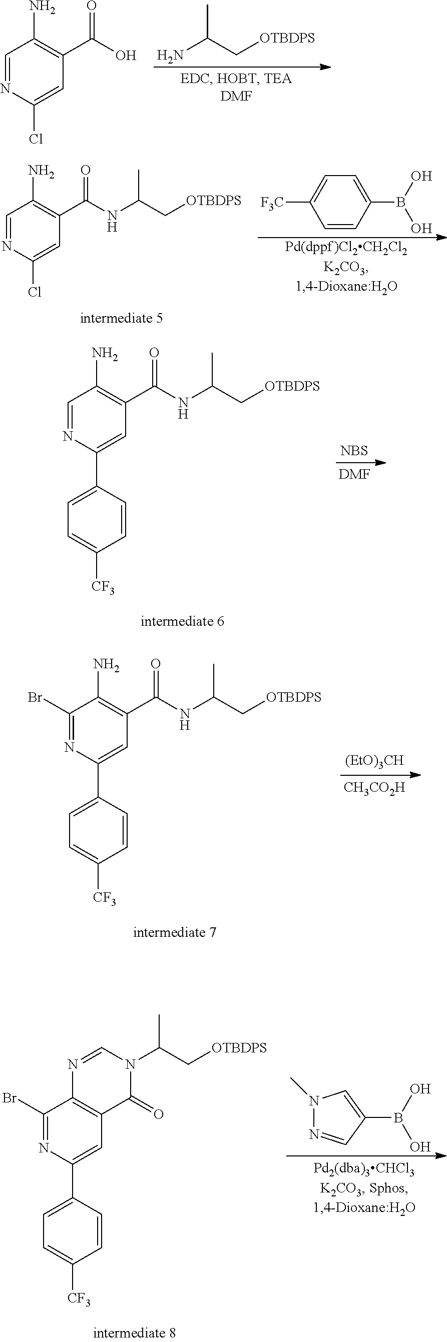

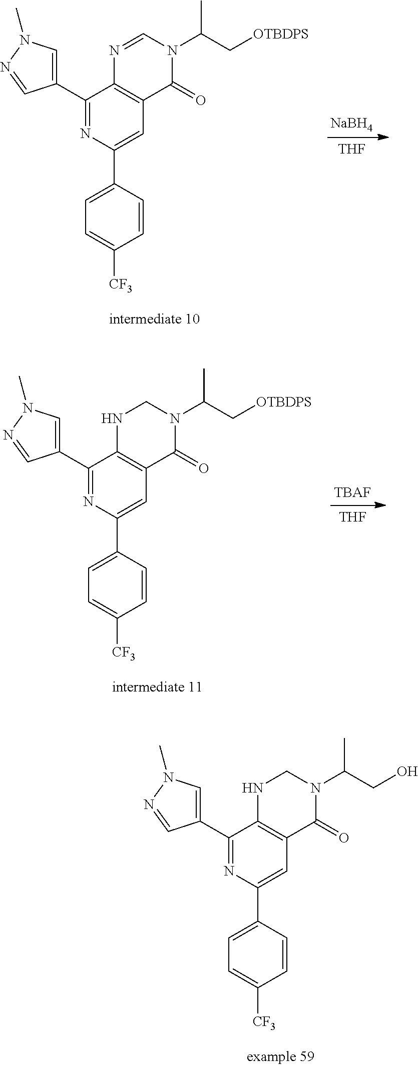

### Intermediate 10. 3-(1-((tert-butyldiphenylsilyl)oxy)propan-2-yl)-8-(1-methyl-1H-pyrazol-4-yl)-6-(4-(trifluoromethyl)phenyl)pyrido[3,4-d]pyrimidin-4(3H)-one

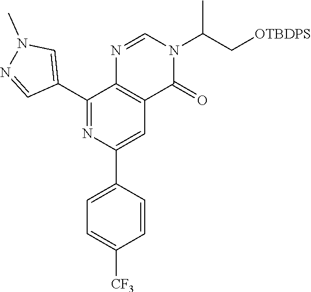

Using (1-methyl-1H-pyrazol-4-yl)boronic acid, the title compound was obtained as described for intermediate 9 of the example 44 (Scheme 2. General procedure B.).

MS (ESI, m/z):668.31 [M+H]⁺

### Intermediate 11. 3-(1-((tert-butyldiphenylsilyl)oxy)propan-2-yl)-8-(1-methyl-1H-pyrazol-4-yl)-6-(4-(trifluoromethyl)phenyl)-2,3-dihydropyrido[3,4-d]pyrimidin-4(1H)-one

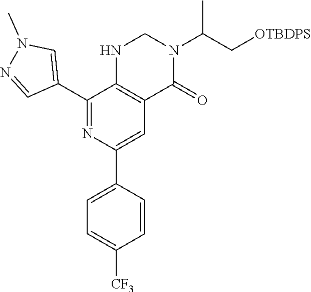

3-(1-((tert-Butyldiphenylsilyl)oxy)propan-2-yl)-8-(1-methyl-1H-pyrazol-4-yl)-6-(4-(trifluoromethyl) phenyl)pyrido[3,4-d]pyrimidin-4(3H)-one (intermediate 10) (23 mg, 0.034 mmol, 1 equiv.) was dissolved in THF (3 mL, 0.011 M) and cooled at 0° C. Then, NaBH₄ (5.21 mg, 0.14 mmol, 4 equiv.) was added to the reaction mixture and stirred for 2h at RT. The reaction mixture was quenched with MeOH (3 mL), diluted with water (3 mL), extracted with EtOAc (5 mL×3), washed with brine (5 mL×3), dried over Na₂SO₄ and glass filtered. The filtrate was evaporated in vacuo and purified by MPLC (silica gel, 0-30% EtOAc/Hexane) to give 20 mg (87% yield) of 3-(1-((tert-butyldiphenylsilyl)oxy)propan-2-yl)-8-(1-methyl-1H-pyrazol-4-yl)-6-(4-(trifluoromethyl)phenyl)-2,3-dihydropyrido[3,4-d]pyrimidin-4(1H)-one (intermediate 11).

MS (ESI, m/z):670.26 [M+H]⁺

### Example 59. 3-(1-hydroxypropan-2-yl)-8-(1-methyl-1H-pyrazol-4-yl)-6-(4-(trifluoromethyl)phenyl)-2,3-dihydropyrido[3,4-d]pyrimidin-4(1H)-one

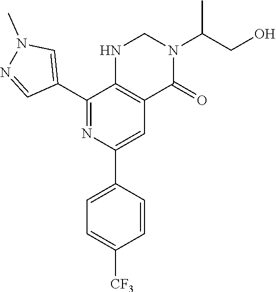 [SMILES: CC(CO)N1CNc2c(cc(-c3ccc(C(F)(F)F)cc3)nc2-c2cnn(C)c2)C1=O]

To a solution of 3-(1-((tert-butyldiphenylsilyl)oxy)propan-2-yl)-8-(1-methyl-1H-pyrazol-4-yl)-6-(4-(trifluoromethyl)phenyl)-2,3-dihydropyrido[3,4-d]pyrimidin-4(1H)-one (intermediate 11) (20 mg, 0.03 mmol, 1 equiv.) in THF (2 mL, 0.01 M) was added TBAF (1.0 M in THF, 30 μL, 0.03 mmol, 1 equiv.) and stirred for 3h at 40° C. The reaction mixture was diluted with water (5 mL) and extracted with EtOAc (5 mL×3), washed with brine (5 mL×3), dried over Na₂SO₄ and glass filtered. The filtrate was evaporated in vacuo and purified by Prep TLC (silica gel Chromatorex KP80805 (NH—SiO₂), 0˜70% EtOAc/Hexane) to give 3.9 mg (30% yield) of 3-(1-hydroxypropan-2-yl)-8-(1-methyl-1H-pyrazol-4-yl)-6-(4-(trifluoromethyl)phenyl)-2,3-dihydropyrido[3,4-d]pyrimidin-4(1H)-one (example 59).

1H-NMR (400 MHz, CDCl₃): δ [ppm]=8.17 (s, 1H), 8.15 (d, J=8.2 Hz, 2H), 8.03 (s, 1H), 7.98 (s, 1H), 7.69 (d, J=8.3 Hz, 2H), 4.74 (dt, J=11.0, 3.8 Hz, 2H), 4.70-4.62 (m, 2H), 3.86 (dd, J=11.6, 3.8 Hz, 1H), 3.73 (d, J=7.8 Hz, 1H), 1.61 (s, 2H), 1.32 (d, J=7.0 Hz, 3H); MS (ESI, m/z):432.01 [M+H]⁺

### Example 60. 6-(4-chlorophenyl)-3-(2,3-dihydroxypropyl)-8-(pyridin-3-yl)pyrido[3,4-d]pyrimidin-4(3H)-one

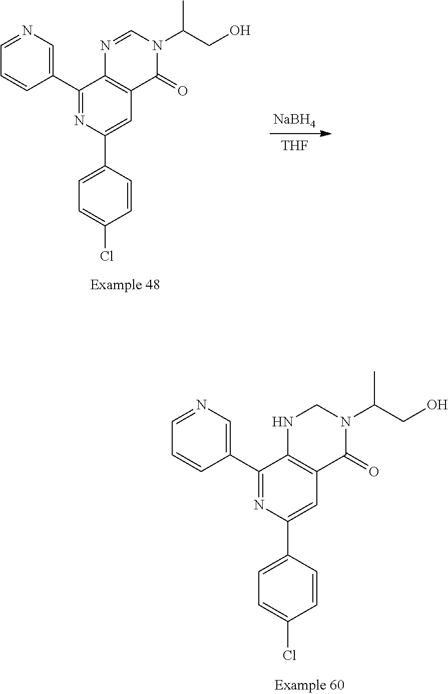 [SMILES: CC(CO)n1cnc2c(-c3cccnc3)nc(-c3ccc(Cl)cc3)cc2c1=O ; "NaBH4" ; CC(CO)N1CNc2c(cc(-c3ccc(Cl)cc3)nc2-c2cccnc2)C1=O ; "THF"]

6-(4-chlorophenyl)-3-(1-hydroxypropan-2-yl)-8-(pyridin-3-yl)pyrido[3,4-d]pyrimidin-4(3H)-one (example 48) (50 mg, 0.127 mmol, 1 equiv.) was dissolved in THF (3 mL, 0.042 M) and cooled at 0° C. Then, NaBH₄ (9.6 mg, 0.255 mmol, 2 equiv.) was added to the reaction mixture and stirred for 2h at rt. The reaction mixture was quenched with MeOH (3 mL), diluted with water (3 mL), extracted with EtOAc (5 mL×3), washed with brine (5 mL×3), dried over Na₂SO₄ and glass filtered. The filtrate was evaporated in vacuo and purified by Prep TLC (silica gel, 0˜10% MeOH/DCM) to give 2.9 mg (5.7% yield) of 6-(4-chlorophenyl)-3-(2,3-dihydroxypropyl)-8-(pyridin-3-yl)pyrido[3,4-d]pyrimidin-4(3H)-one (example 60).

1H-NMR (400 MHz, CDCl₃): a [ppm]=9.04 (s, 1H), 8.70 (s, 1H), 8.24 (s, 1H), 8.12 (d, J=7.9 Hz, 1H), 7.99 (d, J=8.6 Hz, 2H), 7.47 (dd, J=7.6, 5.0 Hz, 1H), 7.41 (d, J=8.5 Hz, 2H), 4.85 (s, 1H), 4.79-4.73 (m, 1H), 4.73-4.61 (m, 2H), 3.85 (dd, J=11.6, 3.8 Hz, 1H), 3.70 (dd, J=11.5, 7.6 Hz, 1H), 1.31 (d, J=7.0 Hz, 3H); MS (ESI, m/z):395.15 [M+H]⁺

### Example 61. 6-(4-chlorophenyl)-3-(3-hydroxyphenyl)-8-(pyridin-3-yl)pyrido[3,4-d]pyrimidin-4(3H)-one

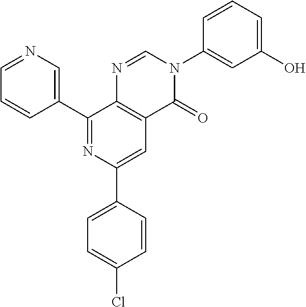 [SMILES: O=c1c2cc(-c3ccc(Cl)cc3)nc(-c3cccnc3)c2ncn1-c1cccc(O)c1]

Using 3-aminophenol, pyridin-3-ylboronic acid and (4-chlorophenyl)boronic acid, the title compound was obtained as described for the example 44 (Scheme 2. General procedure B.).

1H-NMR (400 MHz, CDCl₃): δ [ppm]=9.50 (d, J=1.7 Hz, 1H), 8.72 (dd, J=4.9, 1.5 Hz, 1H), 8.63-8.59 (m, 1H), 8.57 (s, 1H), 8.17 (d, J=8.6 Hz, 2H), 8.15 (s, 1H), 7.54 (dd, J=7.9, 4.9 Hz, 1H), 7.49 (d, J=8.6 Hz, 2H), 7.44 (dd, J=8.1, 8.1 Hz, 1H), 7.04 (dd, J=8.2, 1.8 Hz, 1H), 6.96 (d, J=7.8 Hz, 1H), 6.93-6.90 (m, 1H); MS (ESI, m/z): 427.0 [M+H]⁺

### Example 62. 3-(3-hydroxyphenyl)-8-(pyridin-3-yl)-6-(4-(trifluoromethyl)phenyl)pyrido[3,4-d]pyrimidin-4(3H)-one

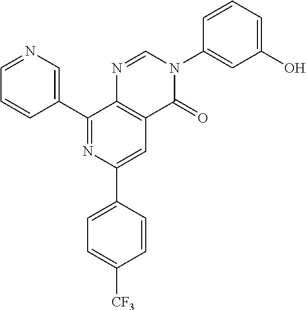 [SMILES: O=c1c2cc(-c3ccc(C(F)(F)F)cc3)nc(-c3cccnc3)c2ncn1-c1cccc(O)c1]

Using 3-aminophenol, pyridin-3-ylboronic acid and (4-(trifluoromethyl)phenyl)boronic acid, the title compound was obtained as described for the example 44 (Scheme 2. General procedure B.).

1H-NMR (400 MHz, CDCl₃): δ [ppm]=9.50 (d, J=2.0 Hz, 1H), 8.74 (dd, J=4.9, 1.6 Hz, 1H), 8.66 (s, 1H), 8.62-8.57 (m, 1H), 8.36 (d, J=8.1 Hz, 2H), 8.20 (s, 1H), 7.79 (d, J=8.3 Hz, 2H), 7.53 (dd, J=8.4, 4.5 Hz, 1H), 7.46 (dd, J=8.1, 8.1 Hz, 1H), 7.04 (dd, J=8.3, 2.3 Hz, 1H), 7.02-6.98 (m, 1H), 6.96-6.92 (m, 1H); MS (ESI, m/z): 461.03 [M+H]⁺

### Example 63. 6-(4-chlorophenyl)-3-(3-hydroxycyclohexyl)-8-(pyridin-3-yl)pyrido[3,4-d]pyrimidin-4(3H)-one

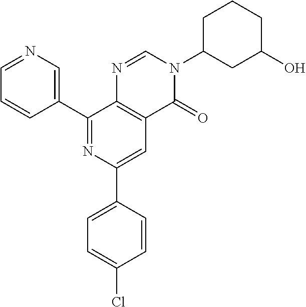 [SMILES: O=c1c2cc(-c3ccc(Cl)cc3)nc(-c3cccnc3)c2ncn1C1CCCC(O)C1]

Using 3-aminocyclohexan-1-ol, pyridin-3-ylboronic acid and (4-chlorophenyl)boronic acid, the title compound was obtained as described for the example 44 (Scheme 2. General procedure B.).

1H NMR (400 MHz, MeOD) δ [ppm]=9.42 (s, 1H), 8.74 (d, J=7.9 Hz, 1H), 8.65 (d, J=4.8 Hz, 1H), 8.60 (s, 1H), 8.50 (s, 1H), 8.26 (d, J=7.6 Hz, 2H), 7.63 (dd, J=8.0, 4.9 Hz, 1H), 7.56 (d, J=7.7 Hz, 2H), 4.79 (t, J=12.5 Hz, 1H), 3.90-3.75 (m, 1H), 2.28 (d, J=12.1 Hz, 1H), 2.07-1.98 (m, 3H), 1.91-1.80 (m, 2H), 1.56 (dd, J=25.7, 13.1 Hz, 1H), 1.38 (dd, J=18.1, 8.6 Hz, 1H); MS (ESI, m/z): 433.11 [M+H]⁺

### Example 64. 6-(4-chlorophenyl)-3-(3-hydroxycyclohexyl)-8-(1-methyl-1H-pyrazol-4-yl)pyrido[3,4-d]pyrimidin-4(3H)-one

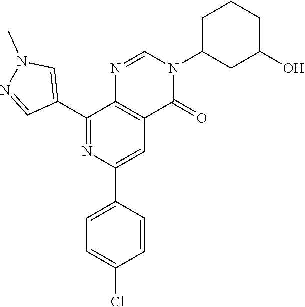 [SMILES: Cn1cc(-c2nc(-c3ccc(Cl)cc3)cc3c(=O)n(C4CCCC(O)C4)cnc23)cn1]

Using 3-aminocyclohexan-1-ol, (1-methyl-1H-pyrazol-4-yl)boronic acid and (4-chlorophenyl)boronic acid, the title compound was obtained as described for the example 44 (Scheme 2. General procedure B.).

1H NMR (400 MHz, MeOD) δ [ppm]=8.80 (s, 1H), 8.58 (s, 1H), 8.52 (s, 1H), 8.34 (s, 1H), 8.23 (d, J=8.6 Hz, 2H), 7.54 (d, J=8.6 Hz, 2H), 4.79 (t, J=12.4 Hz, 1H), 4.02 (s, 3H), 3.84-3.73 (m, 1H), 2.28 (d, J=11.1 Hz, 1H), 2.11-2.01 (m, 2H), 1.91-1.83 (d, J=12.0 Hz, 2H), 1.56 (dd, J=26.7, 13.4 Hz, 1H), 1.42-1.33 (m, 1H); MS (ESI, m/z): 436.10 [M+H]⁺

### Example 65. 3-((1R,3S)-3-hydroxycyclopentyl)-8-(pyridin-3-yl)-6-(6-(trifluoromethyl)pyridin-3-yl)pyrido[3,4-d]pyrimidin-4(3H)-one

Scheme for the preparation of the Compound of Example 65:

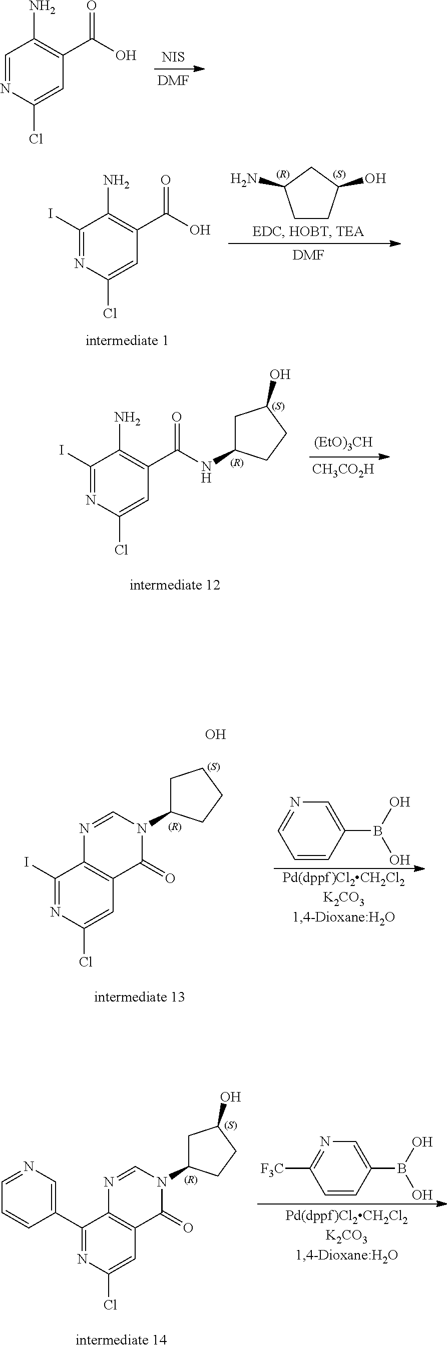

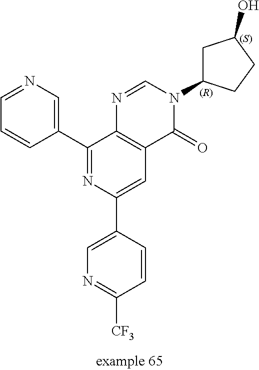

### Intermediate 12. 3-amino-6-chloro-N-((1R,3S)-3-hydroxycyclopentyl)-2-iodoisonicotinamide

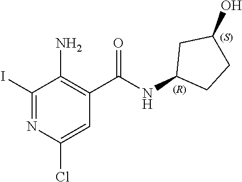

A mixture of 3-amino-6-chloro-2-iodoisonicotinic acid (2 g, 6.70 mmol, 1 equiv.), EDC (1.541 g, 8.04 mmol, 1.2 equiv.), HOBT (1.231 g, 8.04 mmol, 1.2 equiv.) and TEA (1.401 mL, 10.05 mmol, 1.5 equiv.) were dissolved in DMF (50 mL, 0.14 M) and stirred for 5 min. Then (1S,3R)-3-aminocyclopentanol (0.746 g, 7.37 mmol, 1.1 equiv.) was added to the reaction mixture and stirred for 12h at 50° C. The reaction mixture was diluted with water (50 mL), extracted with EtOAc (20 mL×3), washed with brine (20 mL×3), dried over Na₂SO₄ and glass filtered. The filtrate was evaporated in vacuo and purified by MPLC (silica gel, 0-5% MeOH/DCM) to give 2 g (78% yield) of 3-amino-6-chloro-N-((1R,3S)-3-hydroxycyclopentyl)-2-iodoisonicotinamide (intermediate 12).

MS (ESI, m/z): 381.85 [M+H]⁺

### Intermediate 13. 6-chloro-3-((1R,3S)-3-hydroxycyclopentyl)-8-iodopyrido[3,4-d]pyrimidin-4(3H)-one

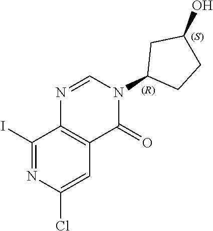

3-amino-6-chloro-N-((1R,3S)-3-hydroxycyclopentyl)-2-iodoisonicotinamide (intermediate 12) (1.5 g, 3.93 mmol, 1 equiv.) was dissolved in a solution of (EtO)₃CH (15 mL, 90 mmol, 23 equiv.) and CH₃CO₂H (15 mL, 262 mmol, 67 equiv.). The reaction mixture was stirred and heated in a Biotage microwave initiator at 150° C. for 1h. The reaction mixture was diluted with water (20 mL), extracted with EtOAc (20 mL×3), washed with brine (10 mL×3), dried over Na₂SO₄ and glass filtered. The filtrate was evaporated in vacuo to give 0.401 g (26% yield) of 6-chloro-3-((1R,3S)-3-hydroxycyclopentyl)-8-iodopyrido[3,4-d]pyrimidin-4(3H)-one (intermediate 13) and used without further purification.

1H NMR (400 MHz, CDCl3): δ [ppm]=8.78 (s, 1H), 8.04 (s, 1H), 5.39-5.27 (m, 1H), 4.59-4.46 (m, 1H), 2.50 (ddd, J=15.8, 11.1, 5.1 Hz, 1H), 2.42-2.33 (m, 1H), 2.18-2.07 (m, 1H), 2.07-1.96 (m, 1H), 1.93-1.86 (m, 1H), 1.86-1.76 (m, 1H); MS (ESI, m/z): 391.89 [M+H]⁺

### Intermediate 14. 6-chloro-3-((1R,3S)-3-hydroxycyclopentyl)-8-(pyridin-3-yl)pyrido[3,4-d]pyrimidin-4(3H)-one

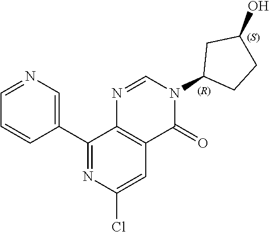

6-chloro-3-((1R,3S)-3-hydroxycyclopentyl)-8-iodopyrido[3,4-d]pyrimidin-4(3H)-one (intermediate 13) (0.360 g, 0.925 mmol, 1 equiv.), pyridin-3-ylboronic acid (0.125 g, 1.018 mmol, 1.1 equiv.), K₂CO₃ (0.384 g, 2.78 mmol, 3 equiv.) and Pd(dppf)Cl₂.CH₂Cl₂ (0.076 g, 9.3 μmol, 0.1 equiv.) were dissolved in 1,4-Dioxane/Water (4 mL/1 mL, 0.06 M. The reaction mixture was stirred and heated in a Biotage microwave initiator at 130° C. for 30 min. The reaction mixture was concentrated under reduced pressure and directly subjected to purification by MPLC (silica gel, 0˜10% MeOH/DCM) to give 0.186 g (60% yield) of 6-chloro-3-((1R,3S)-3-hydroxycyclopentyl)-8-(pyridin-3-yl)pyrido[3,4-d]pyrimidin-4(3H)-one (intermediate 14).

MS (ESI, m/z):343.05 [M+H]⁺

### Example 65. 3-((1R,3S)-3-hydroxycyclopentyl)-8-(pyridin-3-yl)-6-(6-(trifluoromethyl)pyridin-3-yl)pyrido[3,4-d]pyrimidin-4(3H)-one

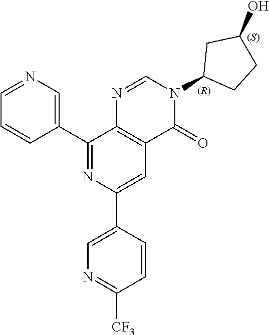

6-chloro-3-((1R,3S)-3-hydroxycyclopentyl)-8-(pyridin-3-yl)pyrido[3,4-d]pyrimidin-4(3H)-one (intermediate 14) (30 mg, 0.088 mmol, 1 equiv.), (6-(trifluoromethyl)pyridin-3-yl)boronic acid (25 mg, 0.131 mmol, 1.5 equiv.), K₂CO₃ (36.6 mg, 0.263 mmol, 3 equiv.) and Pd(dppf)Cl₂.CH₂Cl₂ (7.2 mg, 8.75 μmol, 0.1 equiv.) were dissolved in 1,4-Dioxane/Water (0.7 mL/0.175 mL, 0.1 M. The reaction mixture was stirred and heated in a Biotage microwave initiator at 130° C. for 30 min. The reaction mixture was concentrated under reduced pressure and directly subjected to purification by MPLC (silica gel, 0-10% MeOH/DCM) to give 0.027 g (69% yield) of 3-((1R,3S)-3-hydroxycyclopentyl)-8-(pyridin-3-yl)-6-(6-(trifluoromethyl)pyridin-3-yl)pyrido[3,4-d]pyrimidin-4(3H)-one (example 65).

1H NMR (400 MHz, CDCl3): δ [ppm]=9.55 (d, J=0.9 Hz, 1H), 9.50 (d, J=1.6 Hz, 1H), 8.82-8.70 (m, 3H), 8.67 (s, 1H), 8.62-8.55 (m, 1H), 7.86 (d, J=8.2 Hz, 1H), 7.50 (dd, J=7.8, 4.8 Hz, 1H), 5.44-5.32 (m, 1H), 4.62-4.53 (m, 1H), 2.70 (br, 1H), 2.63-2.53 (m, 1H), 2.48-2.37 (m, 1H), 2.31-2.16 (m, 1H), 2.12-1.96 (m, 2H), 1.94-1.80 (m, 1H); MS (ESI, m/z): 454.18 [M+H]⁺

### Example 66. 3-(2,3-dihydroxypropyl)-8-(pyridin-3-yl)-6-(4-(trifluoromethyl)phenyl)pyrido[3,4-d]pyrimidin-4(3H)-one

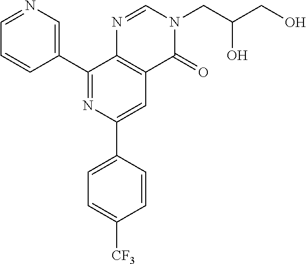 [SMILES: O=c1c2cc(-c3ccc(C(F)(F)F)cc3)nc(-c3cccnc3)c2ncn1CC(O)CO]

Using 3-aminopropane-1,2-diol, pyridin-3-ylboronic acid and (4-(trifluoromethyl)phenyl)boronic acid, the title compound was obtained as described for the example 65 (Scheme 4. General procedure D.).

1H-NMR (400 MHz, CDCl₃): δ [ppm]=9.44 (d, J=1.7 Hz, 1H), 8.78-8.74 (m, 1H), 8.73 (s, 1H), 8.67 (dd, J=4.9, 1.5 Hz, 1H), 8.48 (d, J=8.2 Hz, 2H), 8.39 (s, 1H), 7.87 (d, J=8.4 Hz, 2H), 7.64 (dd, J=8.1, 4.9 Hz, 1H), 4.49 (dd, J=13.6, 3.2 Hz, 1H), 4.10-4.02 (m, 1H), 3.91 (dd, J=13.6, 8.8 Hz, 1H), 3.66 (d, J=5.2 Hz, 2H); MS (ESI, m/z):443.19 [M+H]⁺

### Example 67. 6-(4-chlorophenyl)-3-(2,3-dihydroxypropyl)-8-(pyridin-3-yl)pyrido[3,4-d]pyrimidin-4(3H)-one

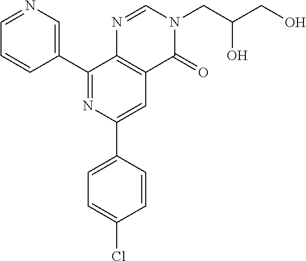 [SMILES: O=c1c2cc(-c3ccc(Cl)cc3)nc(-c3cccnc3)c2ncn1CC(O)CO]

Using 3-aminopropane-1,2-diol, pyridin-3-ylboronic acid and (4-chlorophenyl)boronic acid, the general procedure D (Scheme 4) was followed. In the course of cyclization step c, an additional formylation on one of the two hydroxyl groups was observed by LC-MS analysis. The formylated intermediate was deprotected under the condition of 2M HCl in MeOH for 3h at 40° C. to provide entitled compound of the example 67.

1H-NMR (400 MHz, CDCl₃): δ [ppm]=9.42 (s, 1H), 8.65 (d, J=3.8 Hz, 1H), 8.52 (d, J=7.9 Hz, 1H), 8.45 (s, 1H), 8.20 (s, 1H), 8.12 (d, J=8.5 Hz, 2H), 7.47 (d, J=8.5 Hz, 2H), 7.43 (dd, J=7.8, 4.9 Hz, 1H), 4.33 (dd, J=13.7, 2.8 Hz, 1H), 4.22-4.15 (m, 1H), 4.09 (dd, J=13.6, 6.8 Hz, 1H), 3.72 (ddd, J=16.4, 11.5, 3.9 Hz, 2H); MS (ESI, m/z):409.27 [M+H]⁺

### Example 68. 3-(2,3-dihydroxypropyl)-6-(4-(4-methylpiperazin-1-yl)phenyl)-8-(pyridin-3-yl)pyrido[3,4-d]pyrimidin-4(3H)-one

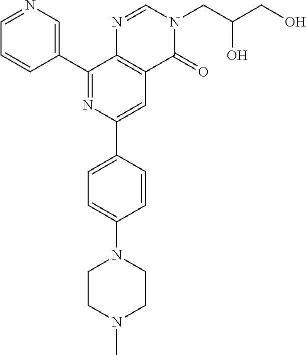 [SMILES: CN1CCN(c2ccc(-c3cc4c(=O)n(CC(O)CO)cnc4c(-c4cccnc4)n3)cc2)CC1]

Using 3-aminopropane-1,2-diol, pyridin-3-ylboronic acid and (4-(4-methylpiperazin-1-yl)phenyl)boronic acid the general procedure D (Scheme 4) was followed. In the course of cyclization Step c), an additional formylation on one of two hydroxyl groups was observed by LC-MS analysis. The formylated intermediate was deprotected under the condition of 2M HCl in MeOH for 3h at 40° C. to provide entitled compound of the example 68.

1H-NMR (400 MHz, CDCl₃): δ [ppm]=9.38 (d, J=1.5 Hz, 1H), 8.70 (dt, J=8.0, 1.9 Hz, 1H), 8.62 (dd, J=4.9, 1.6 Hz, 1H), 8.45 (s, 1H), 8.26 (s, 1H), 8.13 (d, J=8.9 Hz, 2H), 7.60 (dd, J=7.9, 5.0 Hz, 1H), 7.10 (d, J=9.0 Hz, 2H), 4.44 (dd, J=13.6, 3.2 Hz, 2H), 4.08-4.01 (m, 1H), 3.86 (dd, J=13.7, 8.8 Hz, 1H), 3.65 (d, J=5.2 Hz, 2H), 3.36 (t, J=10.0 Hz, 4H), 2.66 (t, J=9.9 Hz, 4H), 2.39 (s, 3H); MS (ESI, m/z):473.17 [M+H]⁺

### Example 69. 3-(1,3-dihydroxypropan-2-yl)-8-(pyridin-3-yl)-6-(4-(trifluoromethyl)phenyl)pyrido[3,4-d]pyrimidin-4(3H)-one

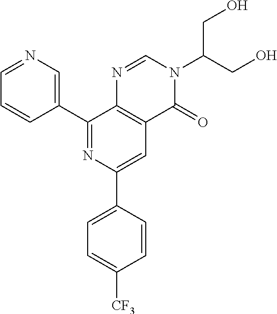 [SMILES: O=c1c2cc(-c3ccc(C(F)(F)F)cc3)nc(-c3cccnc3)c2ncn1C(CO)CO]

Using 2,2-dimethyl-1,3-dioxan-5-amine, pyridin-3-ylboronic acid and (4-(trifluoromethyl)phenyl)boronic acid the general procedure D (Scheme 4) was followed. The intermediate was deprotected under the condition of 2M HCl in MeOH for 3h at 40° C. to provide entitled compound of the example 69.

1H-NMR (400 MHz, CDCl₃): δ [ppm]=9.45 (s, 1H), 8.78-8.74 (m, 1H), 8.71 (s, 1H), 8.66 (dd, J=4.9, 1.6 Hz, 1H), 8.49 (s, 1H), 8.47 (d, J=8.2 Hz, 2H), 7.86 (d, J=8.3 Hz, 2H), 7.64 (ddd, J=8.0, 5.0, 0.8 Hz, 1H), 4.99-4.92 (m, 1H), 4.13 (dd, J=11.9, 7.2 Hz, 2H), 4.00 (dd, J=11.9, 4.9 Hz, 2H); MS (ESI, m/z):443.17 [M+H]⁺

### Example 70. 6-(4-chlorophenyl)-3-(1,3-dihydroxypropan-2-yl)-8-(pyridin-3-yl)pyrido[3,4-d]pyrimidin-4(3H)-one

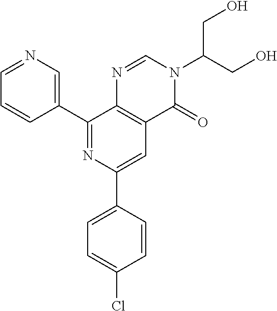 [SMILES: O=c1c2cc(-c3ccc(Cl)cc3)nc(-c3cccnc3)c2ncn1C(CO)CO]

Using 2,2-dimethyl-1,3-dioxan-5-amine, pyridin-3-ylboronic acid and (4-chlorophenyl)boronic acid the general procedure D (Scheme 4) was followed. The intermediate was deprotected under the condition of 2M HCl in MeOH for 3h at 40° C. to provide entitled compound of the example 70.

1H-NMR (400 MHz, CDCl₃): δ [ppm]=9.45 (s, 1H), 8.75-8.72 (m, 1H), 8.65 (dd, J=5.0, 1.6 Hz, 1H), 8.61 (s, 1H), 8.45 (s, 1H), 8.26 (dd, J=6.7, 1.9 Hz, 2H), 7.64-7.61 (m, 1H), 7.56 (dd, J=6.7, 1.9 Hz, 1H), 4.96-4.94 (m, 1H), 4.11 (dd, J=11.9, 7.2 Hz, 2H), 3.99 (dd, J=11.9, 4.9 Hz, 2H); MS (ESI, m/z):409.13 [M+H]⁺

### Example 71. 6-(6-chloropyridin-3-yl)-3-((1R,3S)-3-hydroxycyclopentyl)-8-(pyridin-3-yl)pyrido[3,4-d]pyrimidin-4(3H)-one

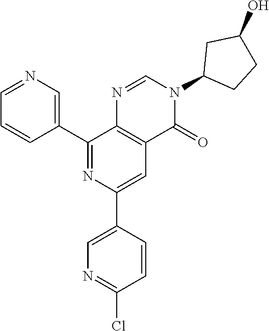 [SMILES: O=c1c2cc(-c3ccc(Cl)nc3)nc(-c3cccnc3)c2ncn1[C@@H]1CC[C@H](O)C1]

Using (1S,3R)-3-aminocyclopentanol, pyridin-3-ylboronic acid and (6-chloropyridin-3-yl)boronic acid, the title compound was obtained as described for the example 65 (Scheme 4. General procedure D.).

1H NMR (400 MHz, CDCl3): δ [ppm]=9.46 (s, 1H), 9.19 (d, J=2.3 Hz, 1H), 8.72 (s, 1H), 8.67 (s, 1H), 8.59-8.52 (m, 2H), 8.50 (dd, J=8.4, 2.5 Hz, 1H), 7.54-7.40 (m, 2H), 5.40-5.24 (m, 1H), 4.60-4.46 (m, 1H), 2.80 (br, 1H), 2.61-2.48 (m, 1H), 2.45-2.33 (m, 1H), 2.28-2.15 (m, 1H), 2.09-1.93 (m, 2H), 1.90-1.79 (m, 1H); MS (ESI, m/z): 420.13 [M+H]⁺

### Example 72. 3-((1R,3S)-3-hydroxycyclopentyl)-8-(pyridin-3-yl)-6-(2-(trifluoromethyl)pyrimidin-5-yl)pyrido[3,4-d]pyrimidin-4(3H)-one, TFA salt

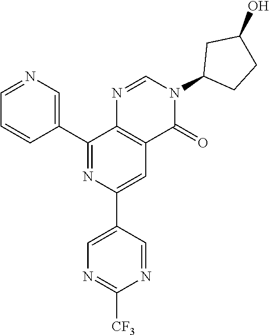 [SMILES: O=c1c2cc(-c3cnc(C(F)(F)F)nc3)nc(-c3cccnc3)c2ncn1[C@@H]1CC[C@H](O)C1]

Using (1S,3R)-3-aminocyclopentanol, pyridin-3-ylboronic acid and (2-(trifluoromethyl)pyrimidin-5-yl)boronic acid, the title compound was obtained as described for the example 65 (Scheme 4. General procedure D.).

1H NMR (400 MHz, MeOD): δ [ppm]=9.98-9.72 (m, 3H), 9.46 (d, J=8.2 Hz, 1H), 9.01-8.83 (m, 3H), 8.13 (dd, J=8.0, 5.8 Hz, 1H), 5.54-5.42 (m, 1H), 4.50-4.43 (m, 1H), 2.62-2.48 (m, 1H), 2.47-2.35 (m, 1H), 2.29-2.07 (m, 2H), 2.05-1.91 (m, 2H); MS (ESI, m/z): 455.10 [M+H]³⁰

### Example 73. 3-((1R,3S)-3-hydroxycyclopentyl)-6-(4-morpholinophenyl)-8-(pyridin-3-yl)pyrido[3,4-d]pyrimidin-4(3H)-one

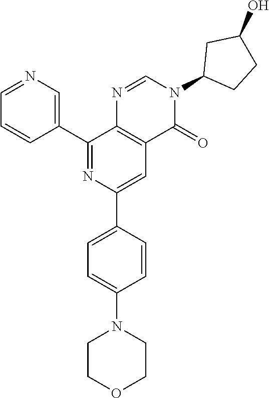 [SMILES: O=c1c2cc(-c3ccc(N4CCOCC4)cc3)nc(-c3cccnc3)c2ncn1[C@@H]1CC[C@H](O)C1]

Using (1S,3R)-3-aminocyclopentanol, pyridin-3-ylboronic acid and (4-morpholinophenyl)boronic acid, the title compound was obtained as described for the example 65 (Scheme 4. General procedure D.).

1H NMR (400 MHz, CDCl3): δ [ppm]=9.46 (d, J=1.6 Hz, 1H), 8.69 (dd, J=4.8, 1.3 Hz, 1H), 8.59-8.52 (m, 1H), 8.49 (s, 1H), 8.48 (s, 1H), 8.19 (d, J=8.9 Hz, 2H), 7.45 (dd, J=7.8, 4.9 Hz, 1H), 7.02 (d, J=8.9 Hz, 2H), 5.26-5.17 (m, 1H), 4.55-4.47 (m, 1H), 3.96-3.79 (m, 4H), 3.36-3.18 (m, 4H), 2.88 (d, J=3.3 Hz, 1H), 2.59-2.49 (m, 1H), 2.38-2.23 (m, 2H), 2.07-1.95 (m, 2H), 1.88-1.78 (m, 1H); MS (ESI, m/z): 470.29 [M+H]⁺

### Example 74. 6-(4′-chloro-[1,1′-biphenyl]-4-yl)-3-(1-hydroxypropan-2-yl)-8-(pyridin-3-yl)pyrido[3,4-d]pyrimidin-4(3H)-one

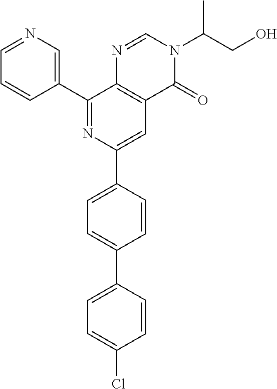 [SMILES: CC(CO)n1cnc2c(-c3cccnc3)nc(-c3ccc(-c4ccc(Cl)cc4)cc3)cc2c1=O]

Using 2-aminopropan-1-ol, pyridin-3-ylboronic acid and (4-chlorophenyl)boronic acid, the general procedure D (Scheme 4) was followed. In the course of Suzuki-coupling step e, the entitled bi-phenyl product was obtained as a side product and purified by Prep HPLC using 20-50-80 0.1% TFA in ACN/Water for elution.

1H-NMR (400 MHz, CDCl3): δ [ppm]=9.46 (d, J=1.7 Hz, 1H), 8.78-8.76 (m, 1H), 8.66 (d, J=4.7 Hz, 2H), 8.46 (s, 1H), 8.38 (d, J=8.5 Hz, 2H), 7.84 (d, J=8.4 Hz, 2H), 7.74 (d, J=8.6 Hz, 2H), 7.64 (dd, J=8.2, 5.4 Hz, 1H), 7.51 (d, J=8.6 Hz, 2H), 5.06-5.01 (m, 1H), 4.00 (dd, J=12.8, 7.1 Hz, 1H), 3.88 (dd, J=11.8, 4.2 Hz, 1H), 1.58 (d, J=7.1 Hz, 3H); MS (ESI, m/z): 469.21 [M+H]⁺

### Example 75. 3-(1-hydroxypropan-2-yl)-6-(4-morpholinophenyl)-8-(pyridin-3-yl)pyrido[3,4-d]pyrimidin-4(3H)-one

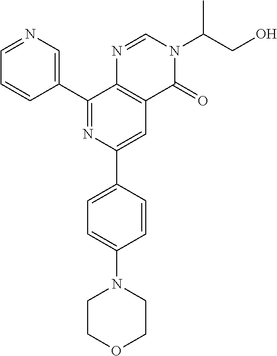 [SMILES: CC(CO)n1cnc2c(-c3cccnc3)nc(-c3ccc(N4CCOCC4)cc3)cc2c1=O]

Using 2-aminopropan-1-ol, pyridin-3-ylboronic acid and (4-morpholinophenyl)boronic acid, the title compound was obtained as described for the example 65 (Scheme 4. General procedure D.).

1H-NMR (400 MHz, CDCl₃): δ [ppm]=9.42 (d, J=1.5 Hz, 1H), 8.75-8.70 (m, 1H), 8.64 (dd, J=4.9, 1.6 Hz, 1H), 8.50 (s, 1H), 8.39 (s, 11H), 8.18 (d, J=9.0 Hz, 2H), 7.62 (dd, J=7.6, 4.6 Hz, 1H), 7.13 (d, J=9.0 Hz, 2H), 5.05-4.99 (m, 1H), 3.99 (dd, J=11.8, 7.0 Hz, 1H), 3.91-3.87 (m, 4H), 3.86 (d, J=4.3 Hz, 1H), 3.31-3.27 (m, 4H), 1.57 (d, J=7.1 Hz, 3H); MS (ESI, m/z):444.21[M+H]⁺

### Example 76. 3-(2-(methylsulfonyl)ethyl)-8-(pyridin-3-yl)-6-(4-(trifluoromethyl)phenyl)pyrido[3,4-d]pyrimidin-4(3H)-one

Scheme for the preparation of the Compound of Example 76:

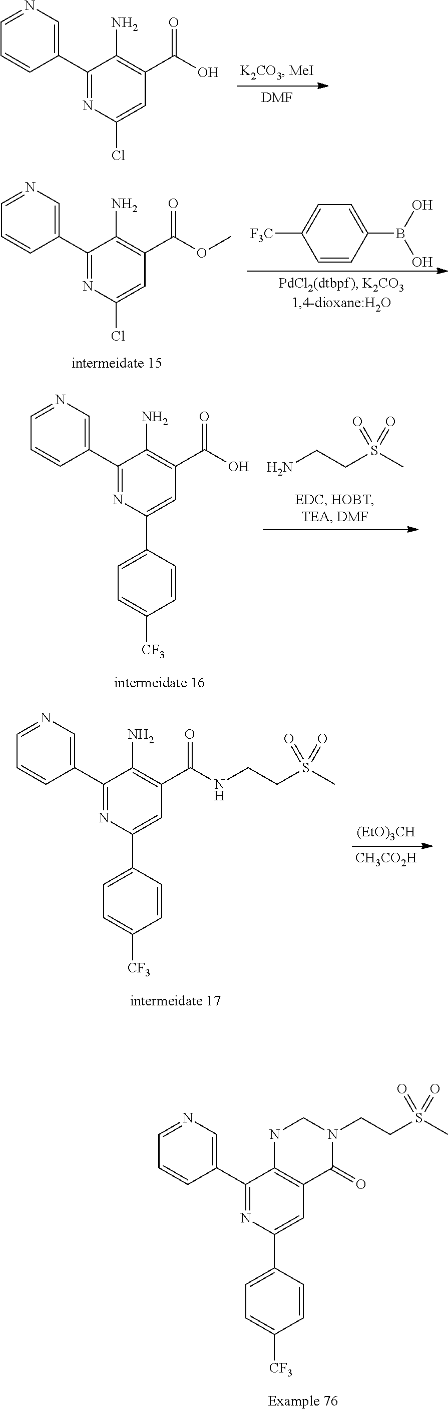

### Intermediate 15. Methyl 3-amino-6-chloro-[2,3′-bipyridine]-4-carboxylate

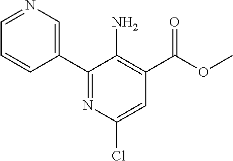 [SMILES: COC(=O)c1cc(Cl)nc(-c2cccnc2)c1N]

A mixture of 3-amino-6-chloro-[2,3′-bipyridine]-4-carboxylic acid (0.5 g, 2.003 mmol, 1 equiv.), K₂CO₃ (0.415 g, 3.00 mmol, 1.5 equiv.) in DMF (20 mL, 0.1 M) and stirred for 5 min. Then Mel (0.426 g, 3.00 mmol, 1.5 equiv.) was added to the reaction mixture and stirred for 3h at RT. The reaction mixture was diluted with water (50 mL), extracted with EtOAc (20 mL×3), washed with brine (20 mL×3), dried over Na₂SO₄ and glass filtered. The filtrate was evaporated in vacuo and purified by MPLC (silica gel, 0-30% EtOAc/Hexane) to give 0.155 g (29% yield) of Methyl 3-amino-6-chloro-[2,3′-bipyridine]-4-carboxylate (intermediate 15). MS (ESI, m/z):264.01 [M+H]⁺

### Intermediate 16. 3-amino-6-(4-(trifluoromethyl)phenyl)-[2,3′-bipyridine]-4-carboxylic acid

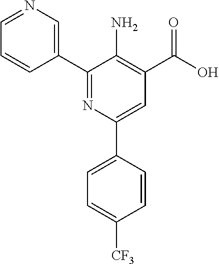 [SMILES: Nc1c(C(=O)O)cc(-c2ccc(C(F)(F)F)cc2)nc1-c1cccnc1]

Methyl 3-amino-6-chloro-[2,3′-bipyridine]-4-carboxylate (intermediate 15) (80 mg, 0.303 mmol, 1 equiv.), (4-(trifluoromethyl)phenyl)boronic acid (86 mg, 0.455 mmol, 1.5 equiv.), K₂CO₃ (126 mg, 0.910 mmol, 3 equiv.) and PdCl₂ (dtbpf) (20 mg, 3 μmol, 0.1 equiv.) were dissolved in 1,4-Dioxane/Water (4 mL/1 mL, 0.06 M. The reaction mixture was stirred and heated in a Biotage microwave initiator at 130° C. for 30 min. The reaction mixture was concentrated under reduced pressure and directly subjected to purification by MPLC (silica gel, 0˜10% MeOH/DCM) to give 76 mg (70% yield) of 3-amino-6-(4-(trifluoromethyl)phenyl)-[2,3′-bipyridine]-4-carboxylic acid (intermediate 16).

MS (ESI, m/z):360.02 [M+H]⁺

### Intermediate 17. 3-amino-N-(2-(methylsulfonyl)ethyl)-6-(4-(trifluoromethyl)phenyl)-[2,3′-bipyridine]-4-carboxamide

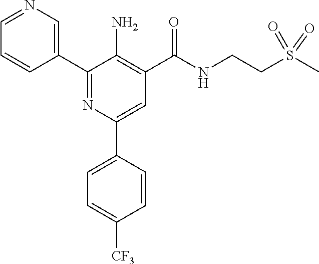 [SMILES: CS(=O)(=O)CCNC(=O)c1cc(-c2ccc(C(F)(F)F)cc2)nc(-c2cccnc2)c1N]

A mixture of 3-amino-6-(4-(trifluoromethyl)phenyl)-[2,3′-bipyridine]-4-carboxylic acid (intermediate 16) (65 mg, 0.181 mmol, 1 equiv.) EDC (42 mg, 0.217 mmol, 1.2 equiv.), HOBT (33 mg, 0.217 mmol, 1.2 equiv.) and TEA (0.038 mL, 0.271 mmol, 1.5 equiv.) were dissolved in DMF (3 mL, 0.13 M) and stirred for 5 min. Then 2-(methylsulfonyl)ethanamine (27 mg, 0.217 mmol, 1.2 equiv.) was added to the reaction mixture and stirred for 12h at 50° C. The reaction mixture was diluted with water (50 mL), extracted with EtOAc (20 mL×3), washed with brine (20 mL×3), dried over Na₂SO₄ and glass filtered. The filtrate was evaporated in vacuo and purified by MPLC (silica gel, 0-5% MeOH/DCM) to give 0.07 g (83% yield) of 3-amino-N-(2-(methylsulfonyl)ethyl)-6-(4-(trifluoromethyl)phenyl)-[2,3′-bipyridine]-4-carboxamide (intermediate 17).

MS (ESI, m/z): 465.09 [M+H]⁺

### Example 76. 3-(2-(methylsulfonyl)ethyl)-8-(pyridin-3-yl)-6-(4-(trifluoromethyl)phenyl)pyrido[3,4-d]pyrimidin-4(3H)-one

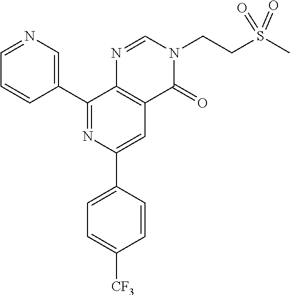 [SMILES: CS(=O)(=O)CCn1cnc2c(-c3cccnc3)nc(-c3ccc(C(F)(F)F)cc3)cc2c1=O]

3-amino-N-(2-(methylsulfonyl)ethyl)-6-(4-(trifluoromethyl)phenyl)-[2,3′-bipyridine]-4-carboxamide (intermediate 17) (70 mg, 0.151 mmol, 1 equiv.) was dissolved in a solution of (EtO)₃CH (1.5 mL, 9.04 mmol, 60 equiv.) and CH₃CO₂H (0.43 mL, 7.54 mmol, 50 equiv.). The reaction mixture was stirred and heated in a Biotage microwave initiator at 150° C. for 1 h. The reaction mixture was diluted with water (20 mL), extracted with EtOAc (20 mL×3), washed with brine (10 mL×3), dried over Na₂SO₄ and glass filtered. The filtrate was evaporated in vacuo and purified by MPLC (silica gel, 0-5% MeOH/DCM) to give 29 mg (40.6% yield) of 3-(2-(methylsulfonyl)ethyl)-8-(pyridin-3-yl)-6-(4-(trifluoromethyl)phenyl)pyrido[3,4-d]pyrimidin-4(3H)-one (example 76).

1H-NMR (400 MHz, CDCl₃): δ [ppm]=9.40 (d, J=1.5 Hz, 1H), 8.74-8.70 (m, 1H), 8.68 (s, 1H), 8.64 (dd, J=4.9, 1.5 Hz, 1H), 8.44 (t, J=4.1 Hz, 3H), 7.84 (d, J=8.3 Hz, 2H), 7.61 (dd, J=8.0, 4.9 Hz, 1H), 4.58 (t, J=6.5 Hz, 2H), 3.74 (t, J=6.5 Hz, 2H), 3.10 (s, 3H). MS (ESI, m/z):475.00 [M]⁺

### Example 77. 6-(4-chlorophenyl)-3-(2-(methylsulfonyl)ethyl)-8-(pyridin-3-yl)pyrido[3,4-d]pyrimidin-4(3H)-one

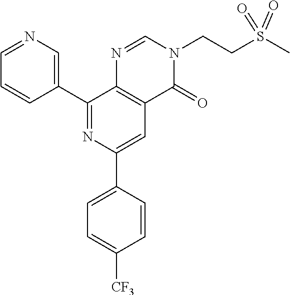 [SMILES: CS(=O)(=O)CCn1cnc2c(-c3cccnc3)nc(-c3ccc(C(F)(F)F)cc3)cc2c1=O]

Using 2-(methylsulfonyl)ethanamine and (4-chlorophenyl)boronic acid, the title compound was obtained as described for the example 76 (Scheme 5. General procedure E.).

1H-NMR (400 MHz, CDCl3): δ=9.39 (s, 1H), 8.70 (d, J=8.0 Hz, 1H), 8.64 (d, J=4.6 Hz, 1H), 8.60 (s, 1H), 8.42 (s, 1H), 8.25 (d, J=8.6 Hz, 2H), 7.61 (dd, J=8.0, 5.0 Hz, 1H), 7.55 (d, J=8.6 Hz, 2H), 4.60-4.50 (m, 2H), 3.73 (t, J=6.5 Hz, 2H), 3.10 (s, 3H); MS (ESI, m/z): 441.07 [M+H]⁺

### Example 78. 3-(1,1-dioxidotetrahydro-2H-thiopyran-4-yl)-6-(4-morpholinophenyl)-8-(pyridin-3-yl)pyrido[3,4-d]pyrimidin-4(3H)-one

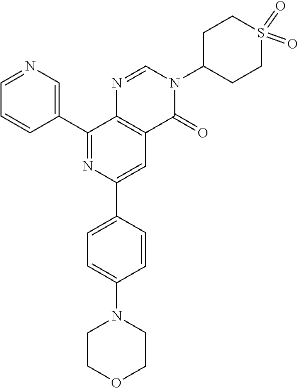 [SMILES: O=c1c2cc(-c3ccc(N4CCOCC4)cc3)nc(-c3cccnc3)c2ncn1C1CCS(=O)(=O)CC1]

Using 4-aminotetrahydro-2H-thiopyran 1,1-dioxide and (4-morpholinophenyl)boronic acid, the title compound was obtained as described for the example 76 (Scheme 5. General procedure E.).

1H-NMR (400 MHz, CDCl3): δ [ppm]=9.44 (d, J=1.8 Hz, 1H), 8.71 (dd, J=4.8, 1.5 Hz, 1H), 8.57-8.49 (m, 1H), 8.45 (s, 1H), 8.19 (d, J=8.9 Hz, 2H), 8.14 (s, 1H), 7.46 (dd, J=7.9, 5.1 Hz, 1H), 7.03 (d, J=9.0 Hz, 2H), 5.19-5.08 (m, 1H), 3.92-3.86 (m, 4H), 3.39-3.20 (m, 8H), 2.70 (dd, J=26.1, 13.0 Hz, 2H), 2.37 (d, J=12.6 Hz, 2H); MS (ESI, m/z): 518.22 [M+H]⁺

### Example 79. 3-(2-(methylsulfonyl)ethyl)-6-(4-morpholinophenyl)-8-(pyridin-3-yl)pyrido[3,4-d]pyrimidin-4(3H)-one

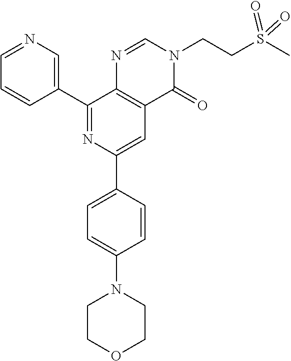 [SMILES: CS(=O)(=O)CCn1cnc2c(-c3cccnc3)nc(-c3ccc(N4CCOCC4)cc3)cc2c1=O]

Using 2-(methylsulfonyl)ethanamine and (4-morpholinophenyl)boronic acid, the title compound was obtained as described for the example 76 (Scheme 5. General procedure E.).

1H-NMR (400 MHz, CDCl3): δ [ppm]=9.45 (d, J=1.6 Hz, 1H), 8.70 (dd, J=4.8, 1.4 Hz, 1H), 8.56 (dt, J=7.9, 1.9 Hz, 1H), 8.43 (s, 1H), 8.17 (d, J=10.2 Hz, 2H), 7.45 (dd, J=7.8, 4.9 Hz, 1H), 7.02 (d, J=8.9 Hz, 2H), 4.51 (t, J=6.2 Hz, 2H), 3.92-3.81 (m, 4H), 3.61 (t, J=6.2 Hz, 2H), 3.34-3.21 (m, 4H), 2.98 (s, 3H); MS (ESI, m/z): 492.21 [M+H]⁺

### Example 80. 3-(1,3-dihydroxypropan-2-yl)-6-(4-morpholinophenyl)-8-(pyridin-3-yl)pyrido[3,4-d]pyrimidin-4(3H)-one

Scheme for the preparation of the Compound of Example 80:

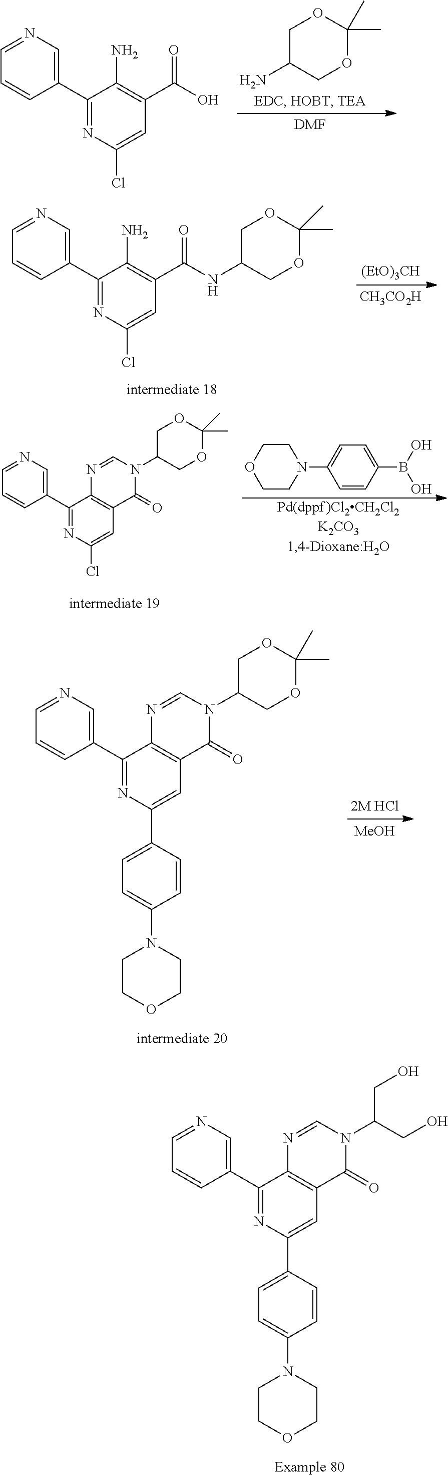

### Intermediate 20. 3-(2,2-dimethyl-1,3-dioxan-5-yl)-6-(4-morpholinophenyl)-8-(pyridin-3-yl)pyrido[3,4-d]pyrimidin-4(3H)-one

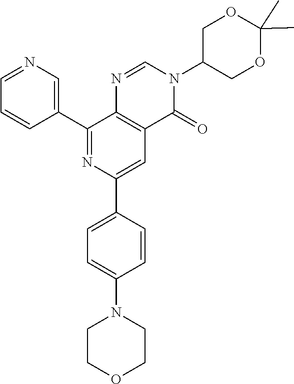 [SMILES: CC1(C)OCC(n2cnc3c(-c4cccnc4)nc(-c4ccc(N5CCOCC5)cc4)cc3c2=O)CO1]

Using 2-dimethyl-1,3-dioxan-5-amine and (4-morpholinophenyl)boronic acid, the title compound was obtained as described for the intermediate 20 (Scheme 6. General procedure F.).

### Example 80. 3-(1,3-dihydroxypropan-2-yl)-6-(4-morpholinophenyl)-8-(pyridin-3-yl)pyrido[3,4-d]pyrimidin-4(3H)-one

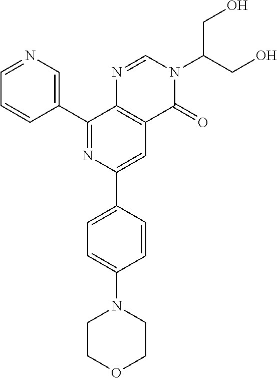 [SMILES: O=c1c2cc(-c3ccc(N4CCOCC4)cc3)nc(-c3cccnc3)c2ncn1C(CO)CO]

Using 2,2-dimethyl-1,3-dioxan-5-amine and (4-morpholinophenyl)boronic acid, the intermediate was deprotected under the conditions of 2M HCl in MeOH for 3h at 40° C. to provided entitled compound of the example 80 (Scheme 6. General procedure F.).

1H NMR (400 MHz, DMSO): δ [ppm]=9.33 (d, J=1.7 Hz, 1H), 8.68 (dd, J=4.8, 1.5 Hz, 1H), 8.54 (dt, J=8.0, 1.8 Hz, 1H), 8.40 (d, J=4.5 Hz, 2H), 8.15 (d, J=8.9 Hz, 2H), 7.58 (dd, J=7.9, 4.8 Hz, 1H), 7.10 (d, J=8.9 Hz, 2H), 4.85-4.75 (m, 1H), 3.91-3.79 (m, 4H), 3.79-3.72 (m, 4H), 3.25-3.21 (m, 4H); MS (ESI, m/z):460.26 [M+H]⁺

### Example 81. (R)-3-(2,3-dihydroxypropyl)-8-(pyridin-3-yl)-6-(4-(trifluoromethyl)phenyl)pyrido[3,4-d]pyrimidin-4(3H)-one

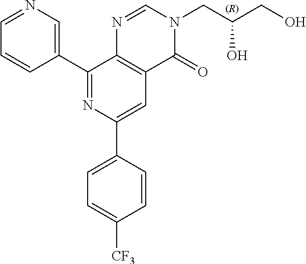

Using (R)-3-aminopropane-1,2-diol, pyridin-3-ylboronic acid (4-(trifluoromethyl)phenyl)boronic acid, the general procedure F (Scheme 6) was followed. In the course of cyclization step c, an additional formylation on one of two hydroxyl groups was observed by LC-MS analysis. The formylated intermediate was deprotected under the condition of 2M HCl in MeOH for 3h at 40° C. to provied entitled compound of the example 81 (Scheme 6. General procedure F.).

1H-NMR (400 MHz, CDCl3): δ [ppm]=9.44 (s, 1H), 8.77-8.71 (m, 1H), 8.71 (s, 1H), 8.66 (d, J=3.8 Hz, 1H), 8.46 (d, J=8.2 Hz, 2H), 8.38 (s, 1H), 7.86 (d, J=8.3 Hz, 2H), 7.63 (dd, J=7.9, 5.0 Hz, 1H), 4.49 (dd, J=13.6, 3.2 Hz, 1H), 4.10-3.99 (m, 1H), 3.91 (dd, J=13.6, 8.8 Hz, 1H), 3.66 (d, J=5.2 Hz, 2H); MS (ESI, m/z):443.45 [M+H]⁺

### Example 82. 3-(2,3-dihydroxypropyl)-6-(4-morpholinophenyl)-8-(pyridin-3-yl)pyrido[3,4-d]pyrimidin-4(3H)-one

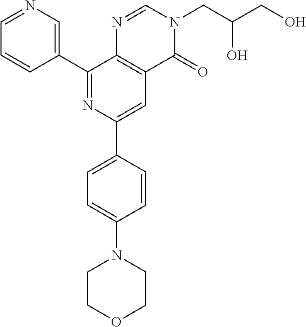 [SMILES: O=c1c2cc(-c3ccc(N4CCOCC4)cc3)nc(-c3cccnc3)c2ncn1CC(O)CO]

Using 3-aminopropane-1,2-diol and (4-morpholinophenyl)boronic acid, the general procedure F (Scheme 6) was followed. In the course of cyclization step c, an additional formylation on one of two hydroxyl groups was observed by LC-MS analysis. The formylated intermediate was deprotected under the conditions of 2M HCl in MeOH for 3h at 40° C. to provided entitled compound of the example 82 (Scheme 6. General procedure F.).

1H-NMR (400 MHz, CDCl3): δ [ppm]=9.45 (d, J=1.8 Hz, 1H), 8.67 (d, J=3.5 Hz, 1H), 8.55 (d, J=7.9 Hz, 1H), 8.44 (s, 1H), 8.17 (d, J=8.8 Hz, 2H), 8.14 (s, 1H), 7.43 (dd, J=7.8, 5.0 Hz, 1H), 7.02 (d, J=8.9 Hz, 2H), 4.30 (d, J=10.2 Hz, 1H), 4.19-4.09 (m, 2H), 3.92-3.87 (m, 4H), 3.76-3.62 (m, 2H), 3.32-3.26 (m, 4H); MS (ESI, m/z):460.19 [M+H]⁺

### Example 83. 2-(6-(4-chlorophenyl)-4-oxo-8-(pyridin-3-yl)pyrido[3,4-d]pyrimidin-3(4H)-yl)propanoic acid, 2,2,2-trifluoroacetic acid salt

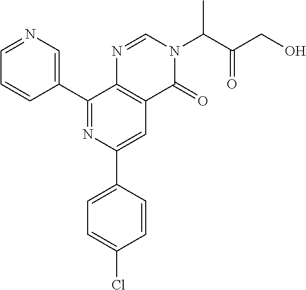 [SMILES: CC(C(=O)CO)n1cnc2c(-c3cccnc3)nc(-c3ccc(Cl)cc3)cc2c1=O]

Using methyl 2-aminopropanoate hydrogen chloride salt, pyridin-3-ylboronic acid and (4-chlorophenyl)boronic acid, the title compound was obtained as described for the example 80 (Scheme 6. General procedure F.).

1H NMR (400 MHz, DMSO): δ [ppm]=9.38 (s, 1H), 8.75 (d, J=3.6 Hz, 1H), 8.64 (d, J=8.0 Hz, 1H), 8.62 (s, 1H), 8.56 (s, 1H), 8.33 (d, J=8.6 Hz, 2H), 7.67 (dd, J=7.8, 4.9 Hz, 1H), 7.61 (d, J=8.6 Hz, 2H), 5.34 (q, J=7.2 Hz, 1H), 1.72 (d, J=7.3 Hz, 3H); MS (ESI, m/z): 407.04 [M+H]⁺

### Example 84. 2-(4-oxo-8-(pyridin-3-yl)-6-(4-(trifluoromethyl)phenyl)pyrido[3,4-d]pyrimidin-3(4H)-yl)propanoic acid, 2,2,2-trifluoroacetic acid salt

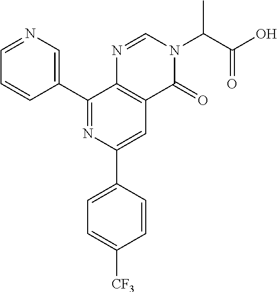 [SMILES: CC(C(=O)O)n1cnc2c(-c3cccnc3)nc(-c3ccc(C(F)(F)F)cc3)cc2c1=O]

Using methyl 2-aminopropanoate hydrogen chloride salt, pyridin-3-ylboronic acid and (4-(trifluoromethyl)phenyl)boronic acid, the title compound was obtained as described for the example 80 (Scheme 6. General procedure F.).

1H NMR (400 MHz, DMSO): δ [ppm]=9.38 (br, 1H), 8.73 (br, 1H), 8.64 (s, 2H), 8.56 (d, J=7.9 Hz, 1H), 8.52 (d, J=8.2 Hz, 2H), 7.91 (d, J=8.4 Hz, 2H), 7.67-7.58 (m, 1H), 5.35 (q, J=7.2 Hz, 1H), 1.72 (d, J=7.3 Hz, 3H); MS (ESI, m/z): 441.07 [M+H]⁺

### Example 86. N-(2-(4-oxo-8-(pyridin-3-yl)-6-(4-(trifluoromethyl)phenyl)pyrido[3,4-d]pyrimidin-3(4H)-yl)propyl)acetamide

Scheme for the preparation of the Compound of Example 86:

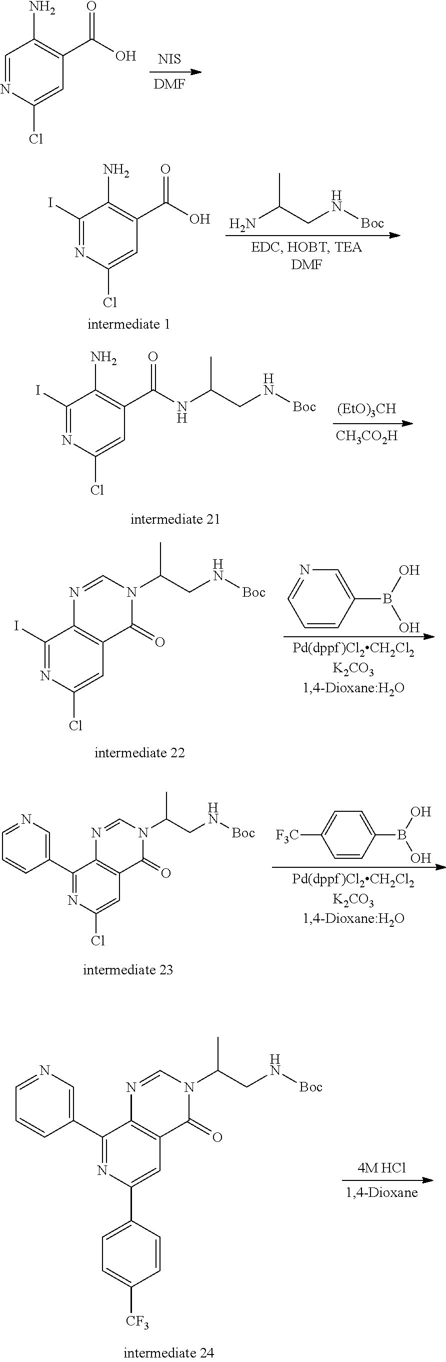

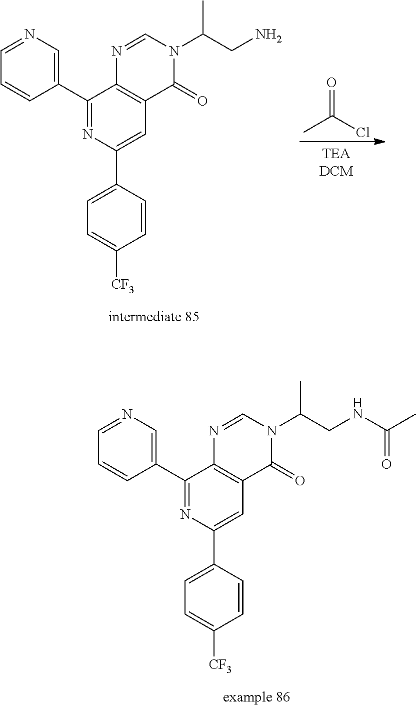

### Intermediate 21. tert-butyl (2-(3-amino-6-chloro-2-iodoisonicotinamido)propyl)carbamate

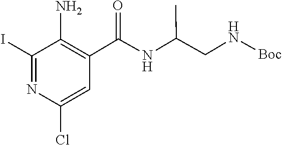 [SMILES: CC(CNC(=O)OC(C)(C)C)NC(=O)c1cc(Cl)nc(I)c1N]

A mixture of 3-amino-6-chloro-2-iodoisonicotinic acid (intermediate 1) (0.8 g, 2.68 mmol, 1 equiv.), EDC (0.617 g, 3.22 mmol, 1.2 equiv.), HOBT (0.493 g, 3.22 mmol, 1.2 equiv.) and TEA (0.560 mL, 4.02 mmol, 1.5 equiv.) were dissolved in DMF (13.4 mL, 0.2 M) and stirred for 10 min. Then, tert-butyl (2-aminopropyl)carbamate (0.475 mL, 2.68 mmol, 1 equiv.) was added to the reaction mixture and stirred for 12h at 50° C. The reaction mixture was diluted with water (50 mL), extracted with EtOAc (20 mL×3), washed with brine (20 mL×3), dried over Na₂SO₄ and glass filtered. The filtrate was evaporated in vacuo and purified by MPLC (silica gel, 0-50% EtOAc/Hexane) to give 0.372 g (31% yield) of tert-butyl (2-(3-amino-6-chloro-2-iodoisonicotinamido)propyl)carbamate (intermediate 21).

1H NMR (400 MHz, CDCl₃): δ=7.71 (d, J=5.1 Hz, 1H), 7.29 (s, 1H), 6.17 (br, 2H), 5.01-4.90 (m, 1H), 4.13-4.00 (m, 1H), 3.37-3.19 (m, 2H), 1.45 (s, 9H), 1.23 (d, J=6.5 Hz, 3H); MS (ESI, m/z): 455.08 [M+H]⁺

### Intermediate 22. 6-chloro-3-((1R,3S)-3-hydroxycyclopentyl)-8-iodopyrido[3,4-d]pyrimidin-4(3H)-one

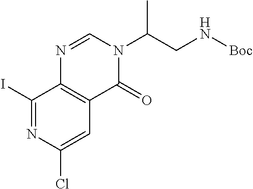 [SMILES: CC(CNC(=O)OC(C)(C)C)n1cnc2c(I)nc(Cl)cc2c1=O]

tert-butyl (2-(3-amino-6-chloro-2-iodoisonicotinamido)propyl)carbamate (intermediate 21) (0.372 g, 0.818 mmol, 1 equiv.) was dissolved in a solution of (EtO)₃CH (1.226 mL, 7.36 mmol, 9 equiv.) and CH₃CO₂H (1.226 mL, 20.18 mmol, 25 equiv.). The reaction mixture was stirred and heated in a Biotage microwave initiator at 150° C. for 1 h. The reaction mixture was diluted with water (20 mL), extracted with EtOAc (20 mL×3), washed with brine (10 mL×3), dried over Na₂SO₄ and glass filtered. The filtrate was evaporated in vacuo and purified by MPLC (silica gel, 0-50% EtOAc/Hexane) to give 0.135 g (36% yield) of tert-butyl (2-(6-chloro-8-iodo-4-oxopyrido[3,4-d]pyrimidin-3(4H)-yl)propyl)carbamate (intermediate 22) and used without further purification.

1H NMR (400 MHz, CDCl₃): δ=8.16 (s, 1H), 8.01 (s, 1H), 4.98-4.87 (m, 1H), 4.76-4.68 (m, 1H), 3.53 (t, J=6.2 Hz, 2H), 1.54 (d, J=7.2 Hz, 3H), 1.32 (s, 9H); MS (ESI, m/z): 465.07 [M+H]⁺

### Intermediate 23. tert-butyl (2-(6-chloro-4-oxo-8-(pyridin-3-yl)pyrido[3,4-d]pyrimidin-3(4H)-yl)propyl)carbamate

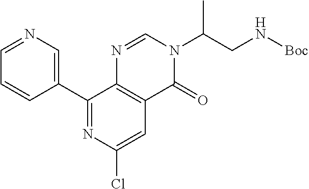 [SMILES: CC(CNC(=O)OC(C)(C)C)n1cnc2c(-c3cccnc3)nc(Cl)cc2c1=O]

tert-butyl (2-(6-chloro-8-iodo-4-oxopyrido[3,4-d]pyrimidin-3(4H)-yl)propyl)carbamate (intermediate 22) (0.123 g, 0.265 mmol, 1 equiv.), pyridin-3-ylboronic acid (0.036 g, 0.291 mmol, 1.1 equiv.), K₂CO₃ (0.110 g, 0.794 mmol, 3 equiv.) and Pd(dppf)Cl₂.CH₂Cl₂ (0.022 g, 0.026 mmol, 0.1 equiv.) were dissolved in 1,4-Dioxane/Water (2.1 mL/0.53 mL, 0.1 M). The reaction mixture was stirred and heated in a Biotage microwave initiator at 130° C. for 30 min. The reaction mixture was diluted with water (10 mL), extracted with EtOAc (10 mL×3), washed with brine (10 mL×3), dried over Na₂SO₄ and glass filtered. The filtrate was evaporated in vacuo and purified by MPLC (silica gel, 0-5% MeOH/DCM) to give 0.093 g (84% yield) of tert-butyl (2-(6-chloro-4-oxo-8-(pyridin-3-yl)pyrido[3,4-d]pyrimidin-3(4H)-yl)propyl)carbamate (intermediate 23).

1H NMR (400 MHz, CDCl₃): δ=9.37 (d, J=1.8 Hz, 1H), 8.70 (dd, J=4.8, 1.5 Hz, 1H), 8.44 (dt, J=8.0, 1.8 Hz, 1H), 8.13 (s, 1H), 8.09 (s, 1H), 7.44 (dd, J=7.9, 4.8 Hz, 1H), 5.02-4.91 (m, 1H), 4.81-4.72 (m, 1H), 3.66-3.47 (m, 2H), 1.56 (d, J=7.1 Hz, 3H), 1.29 (s, 9H); MS (ESI, m/z): 416.18 [M+H]⁺

### Intermediate 24. tert-butyl (2-(4-oxo-8-(pyridin-3-yl)-6-(4-(trifluoromethyl)phenyl)pyrido[3,4-d]pyrimidin-3(4H)-yl)propyl)carbamate

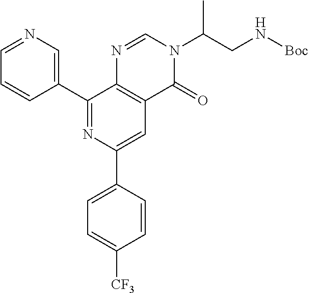 [SMILES: CC(CNC(=O)OC(C)(C)C)n1cnc2c(-c3cccnc3)nc(-c3ccc(C(F)(F)F)cc3)cc2c1=O]

tert-butyl (2-(6-chloro-4-oxo-8-(pyridin-3-yl)pyrido[3,4-d]pyrimidin-3(4H)-yl)propyl)carbamate (intermediate 23) (30 mg, 0.072 mmol, 1 equiv.), (4-(trifluoromethyl)phenyl)boronic acid (15.1 mg, 0.079 mmol, 1.1 equiv.), K₂CO₃ (29.9 mg, 0.216 mmol, 3 equiv.) and Pd(dppf)Cl₂.CH₂Cl₂ (5.9 mg, 7.21 μmol, 0.1 equiv.) were dissolved in 1,4-Dioxane/Water (5.8 mL/1.4 mL, 0.1 M). The reaction mixture was stirred and heated in a Biotage microwave initiator at 130° C. for 30 min. The reaction mixture was diluted with water (10 mL), extracted with EtOAc (10 mL×3), washed with brine (5 mL×3), dried over Na₂SO₄ and glass filtered. The filtrate was evaporated in vacuo and purified by MPLC (silica gel, 0-10% MeOH/DCM) to give 29 mg (76% yield) of tert-butyl (2-(4-oxo-8-(pyridin-3-yl)-6-(4-(trifluoromethyl)phenyl)pyrido[3,4-d]pyrimidin-3(4H)-yl)propyl)carbamate (intermediate 24).

1H-NMR (400 MHz, CDCl₃): δ=9.53 (s, 1H), 8.75 (d, J=3.8 Hz, 1H), 8.63-8.57 (m, 2H), 8.34 (d, J=8.2 Hz, 2H), 8.11 (s, 1H), 7.78 (d, J=8.3 Hz, 2H), 7.50 (dd, J=7.6, 5.0 Hz, 1H), 5.05-4.95 (m, 1H), 4.82-4.74 (m, 1H), 3.69-3.51 (m, 2H), 1.58 (d, J=8.0 Hz, 3H), 1.29 (s, 9H); MS (ESI, m/z): 526.22 [M+H]⁺

### Example 85. 3-(1-aminopropan-2-yl)-8-(pyridin-3-yl)-6-(4-(trifluoromethyl)phenyl)pyrido[3,4-d]pyrimidin-4(3H)-one

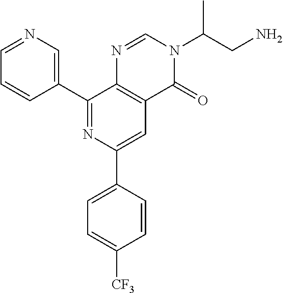 [SMILES: CC(CN)n1cnc2c(-c3cccnc3)nc(-c3ccc(C(F)(F)F)cc3)cc2c1=O]

Tert-butyl (2-(6-(4-chlorophenyl)-4-oxo-8-(pyridin-3-yl)pyrido[3,4-d]pyrimidin-3(4H)-yl)propyl)carbamate (intermediate 24) (29 mg, 0.055 mmol, 1 equiv.) was dissolved in 4M HCl in 1,4-Dioxane (138 μl, 4.0 M, 10 equiv.). The reaction mixture was stirred for 30 min at RT. The reaction mixture was concentrated under reduced pressure and it was diluted with DCM (10 mL). This solution was carefully basified by 1M NaOH solution until the pH of the aqueous layer reached 7. Aqueous layer was further extracted with DCM (10 mL×2), and the combined organic layer was dried over Na₂SO₄ and glass filtered. The filtrate was evaporated in vacuo and purified by MPLC (0˜10% MeOH/DCM) to give 12.2 mg (51% yield) of 3-(1-aminopropan-2-yl)-8-(pyridin-3-yl)-6-(4-(trifluoromethyl)phenyl[3,4-d]pyrimidin-4(3H)-one (example 85).

1H-NMR (400 MHz, CDCl₃): δ [ppm]=9.47 (d, J=1.7 Hz, 1H), 8.72 (dd, J=4.8, 1.6 Hz, 1H), 8.61 (d, J=2.7 Hz, 1H), 8.57 (dt, J=7.9, 1.9 Hz, 1H), 8.35 (d, J=8.2 Hz, 2H), 8.31 (s, 1H), 7.78 (d, J=8.3 Hz, 2H), 7.47 (dd, J=7.8, 4.9 Hz, 1H), 5.09-4.98 (m, 1H), 3.20-3.08 (m, 2H), 1.55 (d, J=7.1 Hz, 3H); MS (ESI, m/z): 426.15 [M+H]⁺

### Example 86. N-(2-(4-oxo-8-(pyridin-3-yl)-6-(4-(trifluoromethyl)phenyl)pyrido[3,4-d]pyrimidin-3(4H)-yl)propyl)acetamide

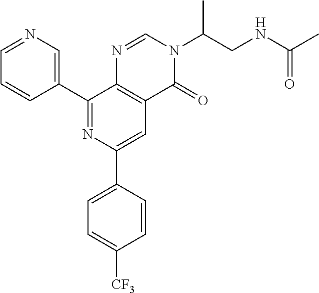 [SMILES: CC(=O)NCC(C)n1cnc2c(-c3cccnc3)nc(-c3ccc(C(F)(F)F)cc3)cc2c1=O]

3-(1-aminopropan-2-yl)-8-(pyridin-3-yl)-6-(4-(trifluoromethyl)phenyl[3,4-d]pyrimidin-4(3H)-one (example 85) (9.8 mg, 0.023 mmol, 1 equiv.) and TEA (3.85 μl, 0.028 mmol, 1.2 equiv.) were dissolved in DCM (115 μl, 0.2 M) and stirred for 5 min. Then, Acetyl chloride (1.97 μl, 0.028 mmol, 1.2 equiv.) was added to the reaction mixture and stirred for 30 min at RT. The reaction mixture was diluted with water (5 mL), extracted with DCM (5 mL×3), washed with brine (5 mL×3), dried over Na₂SO₄ and glass filtered. The filtrate was evaporated in vacuo and purified by MPLC (silica gel, 0-10% MeOH/DCM) to give 4.2 mg (38% yield) of N-(2-(4-oxo-8-(pyridin-3-yl)-6-(4-(trifluoromethyl)phenyl)pyrido[3,4-d]pyrimidin-3(4H)-yl)propyl)acetamide (example 86).

1H NMR (400 MHz, CDCl₃): δ [ppm]=9.46 (d, J=1.7 Hz, 1H), 8.71 (dd, J=4.8, 1.5 Hz, 1H), 8.59-8.54 (m, 2H), 8.33 (d, J=8.2 Hz, 2H), 8.14 (s, 1H), 7.77 (d, J=8.3 Hz, 2H), 7.46 (dd, J=7.8, 4.7 Hz, 1H), 5.94 (t, J=5.9 Hz, 1H), 5.06-4.96 (m, 1H), 3.89-3.77 (m, 1H), 3.71-3.61 (m, 1H), 1.92 (s, 3H), 1.60 (d, J=7.1 Hz, 3H); MS (ESI, m/z): 468.20 [M+H]⁺

### Example 87. 3-(1-aminopropan-2-yl)-6-(4-chlorophenyl)-8-(pyridin-3-yl)pyrido[3,4-d]pyrimidin-4(3H)-one

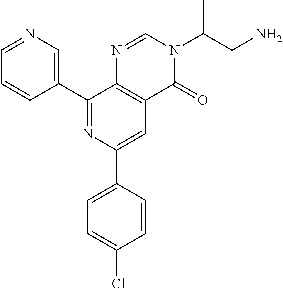 [SMILES: CC(CN)n1cnc2c(-c3cccnc3)nc(-c3ccc(Cl)cc3)cc2c1=O]

Using tert-butyl (2-aminopropyl)carbamate, pyridin-3-ylboronic acid and (4-chlorophenyl)boronic acid, the title compound was obtained as described for the example 85 (Scheme 7. General procedure G.).

1H NMR (400 MHz, MeOD): δ [ppm]=9.41 (d, J=1.7 Hz, 1H), 8.72 (dt, J=8.0, 1.9 Hz, 1H), 8.63 (dd, J=4.9, 1.5 Hz, 1H), 8.58 (s, 1H), 8.39 (s, 1H), 8.23 (d, J=8.6 Hz, 2H), 7.61 (dd, J=8.2, 4.7 Hz, 1H), 7.53 (d, J=8.6 Hz, 2H), 5.00-4.91 (m, 1H), 2.36-2.28 (m, 1H), 2.23-2.13 (m, 1H), 1.58 (d, J=7.0 Hz, 3H); MS (ESI, m/z): 392.14 [M+H]⁺

### Example 88. N-(2-(6-(4-chlorophenyl)-4-oxo-8-(pyridin-3-yl)pyrido[3,4-d]pyrimidin-3(4H)-yl)propyl)acetamide

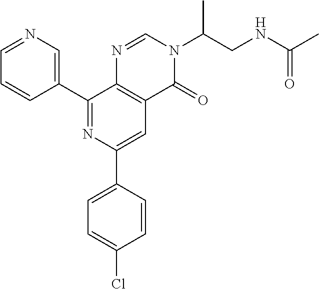 [SMILES: CC(=O)NCC(C)n1cnc2c(-c3cccnc3)nc(-c3ccc(Cl)cc3)cc2c1=O]

Using tert-butyl (2-aminopropyl)carbamate, pyridin-3-ylboronic acid, (4-chlorophenyl)boronic acid and acetyl chloride, the title compound was obtained as described for the example 86 (Scheme 7. General procedure G).

1H NMR (400 MHz, MeOD): δ [ppm]=9.41 (s, 1H), 8.71 (dt, J=8.0, 1.8 Hz, 1H), 8.63 (d, J=3.5 Hz, 1H), 8.57 (s, 1H), 8.35 (s, 1H), 8.23 (d, J=8.6 Hz, 2H), 7.61 (dd, J=7.9, 4.9 Hz, 1H), 7.54 (d, J=8.6 Hz, 2H), 5.08-4.98 (m, 1H), 3.70-3.63 (m, 2H), 1.84 (s, 3H), 1.59 (d, J=7.1 Hz, 3H); MS (ESI, m/z): 434.16 [M+H]⁺

### Example 89. N-(2-(6-(4-chlorophenyl)-4-oxo-8-(pyridin-3-yl)pyrido[3,4-d]pyrimidin-3(4H)-yl)propyl)methanesulfonamide

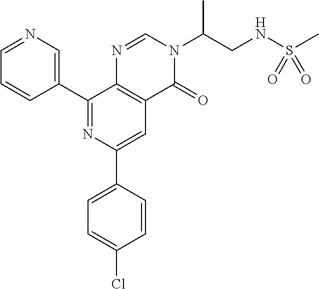 [SMILES: CC(CNS(C)(=O)=O)n1cnc2c(-c3cccnc3)nc(-c3ccc(Cl)cc3)cc2c1=O]

Using tert-butyl (2-aminopropyl)carbamate, pyridin-3-ylboronic acid, (4-chlorophenyl)boronic acid and methanesulfonyl chloride, the title compound was obtained as described for the example 86 (Scheme 7. General procedure G.).

1H NMR (400 MHz, CDCl₃): δ [ppm]=9.35 (d, J=1.5 Hz, 1H), 8.64 (dd, J=4.7, 1.3 Hz, 1H), 8.46 (dt, J=8.0, 1.9 Hz, 1H), 8.30 (s, 1H), 8.14 (s, 1H), 8.03 (d, J=8.7 Hz, 2H), 7.42 (d, J=8.7 Hz, 2H), 7.41-7.37 (m, 1H), 6.07 (t, J=6.5 Hz, 1H), 4.90-4.79 (m, 1H), 3.70-3.59 (m, 1H), 3.50-3.40 (m, 1H), 2.99 (s, 3H), 1.56 (s, 3H); MS (ESI, m/z): 470.11 [M+H]⁺

### Example 90. 3-(1-aminopropan-2-yl)-6-(4-morpholinophenyl)-8-(pyridin-3-yl)pyrido[3,4-d]pyrimidin-4(3H)-one

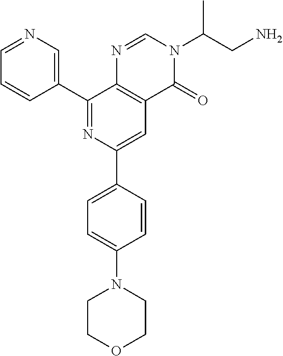 [SMILES: CC(CN)n1cnc2c(-c3cccnc3)nc(-c3ccc(N4CCOCC4)cc3)cc2c1=O]

Using tert-butyl (2-aminopropyl)carbamate, pyridin-3-ylboronic acid and (4-morpholinophenyl)boronic acid, the title compound was obtained as described for the example 85 (Scheme 7. General procedure G.).

1H NMR (400 MHz, MeOD): δ [ppm]=9.39 (d, J=1.6 Hz, 1H), 8.70 (dt, J=8.0, 1.9 Hz, 1H), 8.61 (dd, J=4.9, 1.6 Hz, 1H), 8.46 (s, 1H), 8.31 (s, 1H), 8.14 (d, J=8.9 Hz, 2H), 7.59 (dd, J=8.0, 4.9 Hz, 1H), 7.09 (d, J=9.0 Hz, 2H), 4.96-4.88 (m, 1H), 3.86 (t, J=4.8 Hz, 4H), 3.27 (t, J=4.8 Hz, 4H), 3.23-3.19 (m, 1H), 3.10 (dd, J=13.6, 5.1 Hz, 1H), 1.56 (d, J=7.0 Hz, 3H); MS (ESI, m/z): 443.23 [M+H]⁺

### Example 91. N-(2-(6-(4-morpholinophenyl)-4-oxo-8-(pyridin-3-yl)pyrido[3,4-d]pyrimidin-3(4H)-yl)propyl)methanesulfonamide

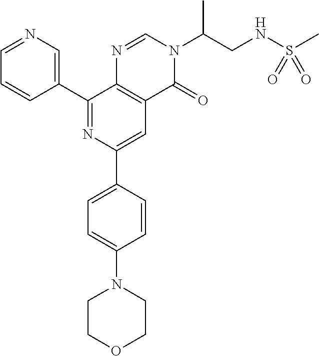 [SMILES: CC(CNS(C)(=O)=O)n1cnc2c(-c3cccnc3)nc(-c3ccc(N4CCOCC4)cc3)cc2c1=O]

Using tert-butyl (2-aminopropyl)carbamate, pyridin-3-ylboronic acid, (4-morpholinophenyl)boronic acid and methanesulfonyl chloride, the title compound was obtained as described for the example 86 (Scheme 7. General procedure G.).

1H NMR (400 MHz, MeOD): δ [ppm]=9.37 (s, 1H), 8.68 (dt, J=8.0, 1.8 Hz, 1H), 8.61 (d, J=4.6 Hz, 1H), 8.42 (s, 1H), 8.27 (s, 1H), 8.11 (d, J=8.9 Hz, 2H), 7.58 (dd, J=7.9, 5.0 Hz, 1H), 7.07 (d, J=8.9 Hz, 2H), 4.96-4.90 (m, 1H), 3.86 (t, J=4.8, 4H), 3.63 (dd, J=14.4, 7.9 Hz, 1H), 3.50 (dd, J=14.3, 4.7 Hz, 1H), 3.26 (t, J=4.8, 4H), 2.92 (s, 3H), 1.60 (d, J=7.1 Hz, 3H); MS (ESI, m/z): 521.21 [M+H]⁺

### Example 92. N-(2-(6-(4-morpholinophenyl)-4-oxo-8-(pyridin-3-yl)pyrido[3,4-d]pyrimidin-3(4H)-yl)propyl)acetamide

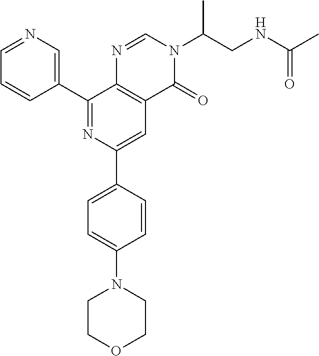 [SMILES: CC(=O)NCC(C)n1cnc2c(-c3cccnc3)nc(-c3ccc(N4CCOCC4)cc3)cc2c1=O]

Using tert-butyl (2-aminopropyl)carbamate, pyridin-3-ylboronic acid, (4-morpholinophenyl)boronic acid and acetyl chloride, the title compound was obtained as described for the example 86 (Scheme 7. General procedure G.).

1H NMR (400 MHz, MeOD): δ [ppm]=9.40 (s, 1H), 8.74-8.69 (m, 1H), 8.62 (d, J=4.8 Hz, 1H), 8.44 (s, 1H), 8.28 (s, 1H), 8.13 (d, J=8.9 Hz, 2H), 7.60 (dd, J=7.9, 4.9 Hz, 1H), 7.09 (d, J=8.9 Hz, 2H), 5.06-4.98 (m, 1H), 3.86 (t, J=4.8 Hz, 4H), 3.66 (t, J=5.8 Hz, 2H), 3.27 (t, J=4.8 Hz, 4H), 1.84 (s, 3H), 1.58 (d, J=7.1 Hz, 3H); MS (ESI, m/z): 485.16 [M+H]⁺

### Example 93. 3-(piperidin-4-yl)-8-(pyridin-3-yl)-6-(4-(trifluoromethyl)phenyl)pyrido[3,4-d]pyrimidin-4(3H)-one

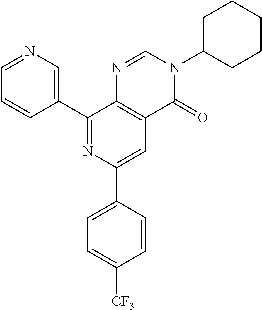 [SMILES: O=c1c2cc(-c3ccc(C(F)(F)F)cc3)nc(-c3cccnc3)c2ncn1C1CCCCC1]

Using tert-butyl 4-aminopiperidine-1-carboxylate, pyridin-3-ylboronic acid and (4-(trifluoromethyl)phenyl)boronic acid, the title compound was obtained as described for the example 85 (Scheme 7. General procedure G.).

1H NMR (400 MHz, MeOD): δ [ppm]=9.42 (d, J=1.6 Hz, 1H), 8.72 (dt, J=8.0, 1.9 Hz, 1H), 8.63 (dd, J=5.0, 1.5 Hz, 1H), 8.62 (s, 1H), 8.44 (s, 1H), 8.41 (d, J=8.2 Hz, 2H), 7.82 (d, J=8.3 Hz, 2H), 7.61 (dd, J=8.0, 4.9 Hz, 1H), 4.85-4.78 (m, 1H), 3.26 (d, J=12.9 Hz, 2H), 2.82 (td, J=13.0, 2.8 Hz, 2H), 2.12-2.01 (m, 4H); MS (ESI, m/z): 452.19 [M+H]⁺

### Example 94. 6-(4-chlorophenyl)-3-(1-(methylsulfonyl)piperidin-4-yl)-8-(pyridin-3-yl)pyrido[3,4-d]pyrimidin-4(3H)-one

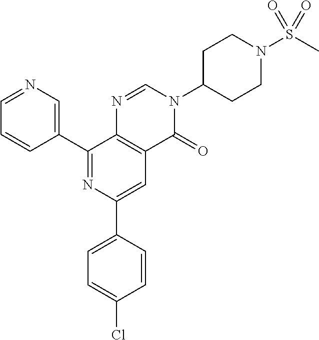 [SMILES: CS(=O)(=O)N1CCC(n2cnc3c(-c4cccnc4)nc(-c4ccc(Cl)cc4)cc3c2=O)CC1]

Using tert-butyl 4-aminopiperidine-1-carboxylate, pyridin-3-ylboronic acid, (4-chlorophenyl)boronic acid and methanesulfonyl chloride, the title compound was obtained as described for the example 86 (Scheme 7. General procedure G.).

1H NMR (400 MHz, CDCl₃): δ [ppm]=9.44 (d, J=1.6 Hz, 1H), 8.72 (dd, J=4.8, 1.6 Hz, 1H), 8.56-8.53 (m, 2H), 8.19 (d, J=8.7 Hz, 2H), 8.17 (s, 1H), 7.50 (d, J=8.7 Hz, 2H), 7.49-7.44 (m, 1H), 5.03-4.93 (m, 1H), 4.09 (d, J=12.4 Hz, 2H), 3.00-2.91 (m, 2H), 2.89 (s, 3H), 2.18-2.11 (m, 4H); MS (ESI, m/z): 496.20 [M+H]⁺

### Example 95. 6-(4-chlorophenyl)-3-(1-(cyclopropylsulfonyl)piperidin-4-yl)-8-(pyridin-3-yl)pyrido[3,4-d]pyrimidin-4(3H)-one

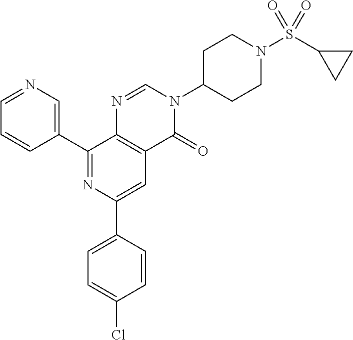 [SMILES: O=c1c2cc(-c3ccc(Cl)cc3)nc(-c3cccnc3)c2ncn1C1CCN(S(=O)(=O)C2CC2)CC1]

Using tert-butyl 4-aminopiperidine-1-carboxylate, pyridin-3-ylboronic acid, (4-chlorophenyl)boronic acid and cyclopropanesulfonyl chloride, the title compound was obtained as described for the example 86 (Scheme 7. General procedure G.).

1H NMR (400 MHz, CDCl₃): δ [ppm]=9.48 (s, 1H), 8.74 (s, 1H), 8.59 (d, J=7.9 Hz, 1H), 8.53 (s, 1H), 8.18 (d, J=9.6 Hz, 3H), 7.50 (d, J=8.6 Hz, 3H), 5.04-4.93 (m, 1H), 4.08 (d, J=12.8 Hz, 2H), 3.14-3.03 (m, 2H), 2.38-2.30 (m, 1H), 2.16-2.07 (m, 4H), 1.25-1.20 (m, 2H), 1.06 (qd, J=5.4, 0.6 Hz, 2H); MS (ESI, m/z): 522.16 [M]⁺

### Example 96. 3-(1-acetylpiperidin-4-yl)-6-(4-chlorophenyl)-8-(pyridin-3-yl)pyrido[3,4-d]pyrimidin-4(3H)-one

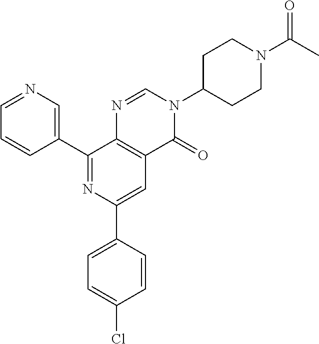 [SMILES: CC(=O)N1CCC(n2cnc3c(-c4cccnc4)nc(-c4ccc(Cl)cc4)cc3c2=O)CC1]

Using tert-butyl 4-aminopiperidine-1-carboxylate, pyridin-3-ylboronic acid, (4-chlorophenyl)boronic acid and acetyl chloride, the title compound was obtained as described for the example 86 (Scheme 7. General procedure G.).

1H NMR (400 MHz, CDCl₃): δ [ppm]=9.43 (d, J=2.0 Hz, 1H), 8.72 (dd, J=4.9, 1.6 Hz, 1H), 8.55-8.51 (m, 2H), 8.19 (d, J=8.6 Hz, 2H), 8.13 (s, 1H), 7.50 (d, J=8.7 Hz, 2H), 7.49-7.45 (m, 1H), 5.11-5.01 (m, 1H), 4.94 (d, J=13.7 Hz, 1H), 4.06 (d, J=13.9 Hz, 1H), 3.33 (t, J=11.8 Hz, 1H), 2.75 (td, J=13.2, 2.1 Hz, 1H), 2.18 (s, 3H), 2.13 (d, J=10.8 Hz, 1H), 2.06 (d, J=10.9 Hz, 1H), 1.98-1.82 (m, 2H); MS (ESI, m/z): 460.19 [M+H]⁺

In Vitro XRE-Luciferase Reporter Assay (In Vitro Assay 1, 2, 3)

AhR activation leads the induction of target gene expression such as CYP1A1 and CYP1B1 by AhR binding to AhR-responsive DNA elements also known as xenobiotics responsive elements (XRE). The assay for measuring AhR activity herein is the luciferase assay using cell lines transfected with luciferase reporter plasmid containing XREs at the upstream of the reporter gene. Cells transfected with XRE-luciferase reporter (XRE-Luc) plasmid drive luciferase activity reflecting activation and inhibition of AhR in the cells. In addition to transfection with XRE-reporter vector, cells were co-transfected with Nano-luciferase reporter gene construct (Nano-Luc) containing constitutively active promoter as internal control. Kynurenine (an endogenous AhR agonist) was used to stimulate cells to test antagonistic properties of the compounds. The half-maximal inhibitory concentration (IC₅₀) or half-maximal effective concentration (EC₅₀) value was calculated using nonlinear regression (four parameters) with Prism8.0 software (GraphPad).

In Vitro Assay 1: Antagonism in Human Cell Line

HepG2 (human hepatoma cell line) cell line with a XRE-luciferase reporter either transiently or stably (Invivogen) were plated in complete medium and incubated at 37° C. in a CO₂ incubator. After 24 hours, cells were treated with kynurenine (50* or 200 μM) alone (negative control) or with test compounds for 6 hours. Luciferase activity was measured with a commercial kit such as the Promega Luciferase kit or other reagents for measuring luciferase activity. Relative luciferase activity (Firefly/Nano-Luc) was used to calculate IC₅₀ values. The relative luciferase activity was further normalized with kynurenine alone group as the maximum control and the vehicle group as the minimum control. The AhR antagonistic potency of the example compounds is listed in Table 1 below. (IC₅₀ values are grouped as A, B, C and D, whereby A: IC₅₀<0.01 μM; B: 0.01<IC₅₀<0.1 μM; C: 0.1<IC₅₀<1.0 μM; D: IC₅₀>1.0 μM)

In Vitro Assay 2: Antagonism in Mouse Cell Line

Hepa1c1c7 (murine liver cancer cell line) cells co-transfected with XRE-Luc and Nano-Luc plasmids were plated in complete medium and incubated overnight at 37° C. in a CO₂ incubator. Following incubation, cells were treated with AhR activating ligands such as kynurenic acid, kynurenine(#) with or without test compounds for 6 hours. Firefly luciferase and Nano-luciferase activity was measured using Nano-glo Luciferase kit (Promega) and relative luciferase activity (Firefly/Nano-Luc) was used to calculate IC₅₀ values. The relative luciferase activity was further normalized with agonists alone group as the maximum control and the vehicle group as the minimum control. The AhR antagonistic potency of the example compounds is listed in Table 1 below. (IC₅₀ Values are grouped as A, B, C and D, whereby A: IC₅₀<0.01 μM; B: 0.01<IC₅₀<0.1 μM; C: 0.1<IC₅₀<1.0 μM; D: IC₅₀>1.0 μM)

In Vitro Assay 3: Agonism in Human Cell Line

HepG2 (human hepatoma cell line) cells co-transfected with XRE-Luc and Nano-Luc plasmids were plated in tryptophan free medium containing 1% of dialyzed fetal bovine serum and incubated overnight at 37° C. in a CO₂ incubator. After 24 hours, cells were treated for 6 hours with test compounds or not. Firefly luciferase and Nano-luciferase activity was measured using Nano-glo Luciferase kit (Promega) and relative luciferase activity (Firefly/Nano-Luc) was used to calculate EC₅₀ values. As a positive control, cells were incubated with TCDD. (EC₅₀ Values are grouped as A, B, C and D, whereby A: EC₅₀<0.1 μM; B: 0.1<EC₅₀<1.0 μM; C: 1.0<EC₅₀<10 μM; D: EC₅₀>10 μM)

TABLE 1
Results of in vitro XRE-luciferase activity assay.

| | Assay 1: | Assay 2: | Assay 3: | |
|---|---|---|---|---|
| | AhR-Luc Human | AhR-Luc Mouse | AhR-Luc Human | |
| | Antagonism | Antagonism | Agonism | |
| Example | (IC₅₀, nM) | (IC₅₀, nM) | (EC₅₀, nM) | |
| 1 | A* | — | >30,000 | (D) |
| 2 | C* | — | — | |
| 3 | D* | — | >30,000 | (D) |
| 4 | D* | — | — | |
| 5 | D* | — | — | |
| 6 | C* | — | — | |
| 7 | D* | — | — | |
| 8 | A* | — | >30,000 | (D) |
| 9 | C* | — | — | |
| 10 | A* | — | >30,000 | (D) |
| 11 | C* | — | — | |
| 12 | A* | — | — | |
| 13 | A* | — | — | |
| 14 | A* | — | >30,000 | (D) |
| 15 | D | — | — | |
| 16 | A | — | — | |
| 17 | D | — | — | |
| 18 | A | — | — | |
| 19 | A | — | — | |
| 20 | D | — | — | |
| 21 | D | — | — | |
| 22 | A | — | — | |
| 23 | A | — | — | |
| 24 | A | — | — | |
| 25 | D | — | — | |
| 26 | B | — | — | |
| 27 | A | — | — | |
| 28 | A | — | — | |
| 29 | A | — | — | |
| 30 | B | — | — | |
| 31 | A | — | — | |
| 32 | A | — | — | |
| 33 | A | — | — | |
| 34 | A | — | — | |
| 35 | A | — | — | |
| 36 | B | — | — | |
| 37 | A | — | — | |
| 38 | A | — | 1675 | (C) |
| 39 | D | — | — | |
| 40 | A | — | — | |
| 41 | D | — | — | |
| 42 | D | — | — | |
| 43 | D | — | — | |
| 44 | A* | — | >30,000 | (D) |
| 45 | A* | — | 0.04279 | (A) |
| 46 | A | A# | >30,000 | (D) |
| 47 | B | — | — | |
| 48 | A* | — | >30,000 | (D) |
| 49 | A* | — | 9658 | (C) |
| 50 | A* | — | 2049 | (C) |
| 51 | A* | — | 701.1 | (B) |
| 52 | A* | — | 289.6 | (B) |
| 53 | D* | — | — | |
| 54 | D* | — | — | |
| 55 | A* | — | 1373 | (C) |
| 56 | A | A | >30,000 | (D) |
| 57 | A* | — | 4479 | (C) |
| 58 | A | — | — | |
| 59 | A* | — | >30,000 | (D) |
| 60 | B | — | — | |
| 61 | A | — | — | |
| 62 | A | — | — | |
| 63 | A | — | — | |
| 64 | B | — | — | |
| 65 | A | — | — | |
| 66 | A | B# | — | |
| 67 | A | — | >30,000 | (D) |
| 68 | D | — | — | |
| 69 | A | — | — | |
| 70 | A | — | — | |
| 71 | B | — | >30,000 | (D) |
| 72 | B | — | — | |
| 73 | A | — | — | |
| 74 | A | — | — | |
| 75 | A | B# | >30,000 | (D) |
| 76 | A | — | >30,000 | (D) |
| 77 | A | — | >30,000 | (D) |
| 78 | A | — | — | |
| 79 | A | — | — | |
| 80 | A | — | — | |
| 81 | A | — | — | |
| 82 | A | — | >30,000 | (D) |
| 83 | C | — | — | |
| 84 | C | — | — | |
| 85 | B | — | — | |
| 86 | B | — | — | |
| 87 | A | — | — | |
| 88 | A | B# | >30,000 | (D) |
| 89 | A | — | >30,000 | (D) |
| 90 | C | — | — | |
| 91 | A | — | — | |
| 92 | B | — | — | |
| 93 | A | — | — | |
| 94 | A | — | >30,000 | (D) |
| 95 | A | — | >30,000 | (D) |
| 96 | A | A | >30,000 | (D) |

(*Cells were treated with kynurenine 50 μM #Cells were treated with kynurenine)

In Vitro Assay 4: Endogenous AhR Activity Assay

HepG2 cells were seeded in 12-well plate (3×10⁵ cells/well). A day after seeding, the cells were treated with TCDD (10 nM) alone or with compounds (123 nM) for 4 hours. Total RNA was extracted using Trizol (Thermo Fisher Scientific). cDNA synthesis and quantitative RT-PCR (qRT-PCR) assays were performed using PrimeScript™ RT Master Mix (TAKARA) and TB Green™ Premix Ex Taq™ II (TAKARA) in accordance with manufacturer's instruction. For the measurement of endogenous AhR activity, relative mRNA levels of CYP1A1 and CYP1B1 were quantitated relative to β(beta)-actin mRNA by the comparative Ct (ΔΔCt) method. The percent inhibitions were calculated according to:

$$( 1 - \frac{\begin{matrix}Relative mRNA level of compound treated group - \\ Relative mRNA level of vehicle group\end{matrix}}{\begin{matrix}Relative mRNA level of TCDD treated group - \\ Relative mRNA level of vehicle group\end{matrix}} ) \times 100 \% = \% inhibition$$

The endogenous AhR antagonistic potency of the example compounds is listed in Table 2 below.

TABLE 2
Results of in vitro endogenous AhR activity assay.

| Compound_ID | CYP1A1 (% Inhibition) | CYP1B1 (% Inhibition) |
|---|---|---|
| Example 16 | 99.58 | 100.37 |
| Example 17 | 90.71 | 99.10 |
| Example 38 | 100.09 | — |
| Example 45 | 99.58 | 100.37 |
| Example 46 | 90.71 | 99.10 |
| Example 51 | 99.20 | — |
| Example 56 | 101.32 | — |
| Example 66 | 100.21 | — |
| Example 75 | 101.45 | — |
| Example 88 | 99.18 | — |
| Example 96 | 99.94 | — |

1. A compound of formula (I), or an enantiomer, diastereomer, racemate, solvate, hydrate, or pharmaceutically acceptable salt thereof: 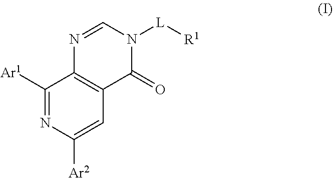
wherein:
Ar¹ and Ar² are independently selected from a group consisting of halo, substituted or unsubstituted mono- or bicyclic C₆₋₁₀ aryl, substituted or unsubstituted mono- or bicyclic C₅₋₁₀ heteroaryl and substituted or unsubstituted mono- or bicyclic C₃₋₁₀ heterocycloalkyl;
L is absent(direct bond), H, halo, cyano, hydroxy, amino, nitro, ether(-O—), thioether(-S—), sulfinyl(-SO—), sulfonyl(-SO₂—), sulfonylamido(-SO₂NR²—), aminosulfonyl(—NR²SO₂—), carbonyl(-(CO)—), amido(-(CO)NR²—), reverse amido(-NR²(CO)—), ester(-(CO)O—), substituted or unsubstituted C₁₋₅ alkyl, substituted or unsubstituted mono- or bicyclic C₃₋₁₀ cycloalkyl, substituted or unsubstituted mono- or bicyclic C₄₋₁₀ heterocycloalkyl, substituted or unsubstituted mono- or bicyclic C₆₋₁₀ aryl and substituted or unsubstituted mono- or bicyclic C₅₋₁₀ heteroaryl;
R¹ is absent(direct bond), H, halo, cyano, hydroxy, amino, NHR³, OR³, phosphate, substituted or unsubstituted C₁₋₃ alkyl phosphate, substituted or unsubstituted C₁₋₅ alkyl, sulfinic acid(-SO—H), sulfonic acid(-SO₂—H), sulfonylamide(-SO₂NR²₂), aminosulfonic acid(—NR²SO₂—H), carboxylic acid(-(CO)—H), carbonyl((-(CO)R²), amide(-(CO)NR²₂), reverse alkyl amide(-NH(CO)—R²), alkyl ester(-(CO)O—R²), sulfonate(-SO₂—R²), C₃₋₁₀ cycloalkyl, C₁₋₅ alkylhydroxy, C₁₋₅ alkenylhydroxy, C₁₋₅ alkynylhydroxy, C₁₋₅ alkylamine, C₁₋₅ alkenylamine, C₁₋₅ alkynylamine, substituted or unsubstituted mono- or bicyclic C₃₋₁₀ heterocycloalkyl and substituted or unsubstituted mono- or bicyclic C₅₋₁₀ heteroaryl;
R² is H, halo, hydroxy, amino, substituted or unsubstituted C₁₋₅ alkyl, substituted or unsubstituted C₁₋₅ alkoxy, substituted or unsubstituted C₃₋₈ cycloalkyl and substituted or unsubstituted C₁₋₅ alkyl carboxylic acid;
R³ is H, substituted or unsubstituted C₁₋₅ alkyl, C₁₋₅ alkylacetyl(alkyl-CO—), C₁₋₅ sulfonylalkyl(alkyl-SO₂—), C₁₋₅ sulfonylamidoalkyl(alkyl-SO₂NR²₂), C₁₋₅ amidoalkyl(alkyl-(CO)NR²₂), C₁₋₅ reverse amidoalkyl(alkyl-NR²(CO)—), substituted or unsubstituted C₁₋₅ alkoxy and substituted or unsubstituted C₁₋₅ alkyl carboxylic acid.

2. The compound, or an enantiomer, diastereomer, racemate, solvate, hydrate, or pharmaceutically acceptable salt thereof according to claim 1,
wherein the Ar¹ and the Ar² is each independently halo, substituted or unsubstituted mono- or bicyclic C₆₋₁₀ aryl, substituted or unsubstituted monocyclic C₅₋₇ heteroaryl comprising one or more hetero atoms selected from the group consisting of N, O and S, or substituted or unsubstituted monocyclic C₅₋₇ heterocycloalkyl comprising one or more hetero atoms selected from the group consisting of N, O and S.

3. The compound, or an enantiomer, diastereomer, racemate, solvate, hydrate, or pharmaceutically acceptable salt thereof according to claim 1,
wherein the Ar¹ and the Ar² is each independently phenyl, monocyclic C₅₋₆ heteroaryl comprising one or two hetero atoms selected from the group consisting of N, O and S, or monocyclic C₅₋₆ heterocycloalkyl comprising one or two hetero atoms selected from the group consisting of N, O and S, which is unsubstituted or substituted with halo, hydroxyl, amino, C₁₋₃ alkyl or C₁₋₃ alkoxy, where C₁₋₃ alkyl or C₁₋₃ alkoxy is unsubstituted or substituted with one to three halo.

4. The compound, or an enantiomer, diastereomer, racemate, solvate, hydrate, or pharmaceutically acceptable salt thereof according to claim 1,
wherein L is absent(direct bond), H, halo, cyano, hydroxy, amino, nitro, ether(-O—), thioether(-S—), sulfinyl(-SO—), sulfonyl(-SO₂—), sulfonylamido(-SO₂NR²—), aminosulfonyl(-NR²SO₂—), carbonyl(-(CO)—), amido(-(CO)NR²—), reverse amido(-NR²(CO)—), ester(-(CO)O—), substituted or unsubstituted mono- or bicyclic C₃₋₈ cycloalkyl, substituted or unsubstituted mono- or bicyclic C₃₋₈ heterocycloalkyl, substituted or unsubstituted mono- or bicyclic C₆₋₁₀ aryl and substituted or unsubstituted mono- or bicyclic C₅₋₈ heteroaryl, wherein the mono- or bicyclic C₃₋₈ heterocycloalkyl and mono- or bicyclic C₅₋₈ heteroaryl comprises one or more heteroatoms selected from the group consisting of N, O and S.

5. The compound, or an enantiomer, diastereomer, racemate, solvate, hydrate, or pharmaceutically acceptable salt thereof according to claim 1,
wherein L is absent(direct bond), H, substituted or unsubstituted C₁₋₅ alkyl, 1,1-dioxydotetrahydrothiopyrane, piperidine, substituted or unsubstituted mono- or bicyclic C₃₋₆ cycloalkyl, where C₁₋₅ alkyl, substituted or unsubstituted mono- or bicyclic C₃₋₆ cycloalkyl is substituted with one or more substituents selected from a group consisting of hydroxyl, halo, haloC₁₋₃ alkyl and C₁₋₃ alkyl.

6. The compound, or an enantiomer, diastereomer, racemate, solvate, hydrate, or pharmaceutically acceptable salt thereof according to claim 1,
wherein R¹ is absent, H, halo, cyano, hydroxy, amino, N(R³)₂, OR³, substituted or unsubstituted C₁₋₄ alkyl, carbonyl((-(CO)R²), C₃₋₈ cycloalkyl, C₁₋₄ alkylhydroxy, C₁₋₄ alkenylhydroxy, C₁₋₄ alkynylhydroxy, C₁₋₄ alkylamine, C₁₋₄ alkenylamine, C₁₋₄ alkynylamine, substituted or unsubstituted mono- or bicyclic C₃₋₈ heterocycloalkyl and substituted or unsubstituted mono- or bicyclic C₅₋₈ heteroaryl, wherein the mono- or bicyclic C₃₋₈ heterocycloalkyl and mono- or bicyclic C₅₋₈ heteroaryl comprises one or more heteroatoms selected from the group consisting of N, O and S.

7. The compound, or an enantiomer, diastereomer, racemate, solvate, hydrate, or pharmaceutically acceptable salt thereof according to claim 1,
wherein R¹ is absent, H, hydroxyl, —NH₂, —NH—C(O)CH₃, —NH—SO₂—CH₃, —C(O)OH, —SO2-CH3, —OC(O)—CH₃, —O—P(═O)(OCH₂CH₃)₂, —C(O)CH₃, or hydroxyl.

8. The compound according to claim 1, which is selected from any one of the compounds 1 to 96, or an enantiomer, diastereomer, racemate, solvate, hydrate, or pharmaceutically acceptable salt thereof:
1. 3-(3-hydroxycyclohexyl)-8-(1-methyl-1H-pyrazol-4-yl)-6-(4-(trifluoromethyl)phenyl)pyrido[3,4-d]pyrimidin-4(3H)-one;
2. 3-(3-hydroxycyclohexyl)-6,8-bis(1-methyl-1H-pyrazol-4-yl)pyrido[3,4-d]pyrimidin-4(3H)-one;
3. 3-(1-hydroxypropan-2-yl)-6,8-bis(1-methyl-1H-pyrazol-4-yl)pyrido[3,4-d]pyrimidin-4(3H)-one;
4. 3-(1-hydroxypropan-2-yl)-6-(1-methyl-1H-pyrazol-4-yl)-8-(4-(trifluoromethyl)phenyl)pyrido[3,4-d]pyrimidin-4(3H)-one;
5. 8-(4-chlorophenyl)-3-(1-hydroxypropan-2-yl)-6-(pyridin-3-yl)pyrido[3,4-d]pyrimidin-4(3H)-one;
6. 3-(1-hydroxypropan-2-yl)-6,8-bis(4-(trifluoromethyl)phenyl)pyrido[3,4-d]pyrimidin-4(3H)-one;
7. 2-(6-chloro-8-(4-chlorophenyl)-4-oxopyrido[3,4-d]pyrimidin-3(4H)-yl)propyl acetate;
8. 3-((1r,4r)-4-hydroxycyclohexyl)-8-(1-methyl-1H-pyrazol-4-yl)-6-(4-(trifluoromethyl)phenyl)pyrido[3,4-d]pyrimidin-4(3H)-one;
9. 3-((1r,4r)-4-hydroxycyclohexyl)-6,8-bis(1-methyl-1H-pyrazol-4-yl)pyrido[3,4-d]pyrimidin-4(3H)-one;
10. 6-(4-chlorophenyl)-3-((1r,4r)-4-hydroxycyclohexyl)-8-(1-methyl-1H-pyrazol-4-yl)pyrido[3,4-d]pyrimidin-4(3H)-one;
11. 3-(2-hydroxypropyl)-6,8-bis(1-methyl-1H-pyrazol-4-yl)pyrido[3,4-d]pyrimidin-4(3H)-one;
12. 3-(2-hydroxypropyl)-8-(1-methyl-1H-pyrazol-4-yl)-6-(4-(trifluoromethyl)phenyl)pyrido[3,4-d]pyrimidin-4(3H)-one;
13. 6-(4-chlorophenyl)-3-(2-hydroxypropyl)-8-(1-methyl-1H-pyrazol-4-yl)pyrido[3,4-d]pyrimidin-4(3H)-one;
14. 3-(2-hydroxypropyl)-8-(1-methyl-1H-pyrazol-4-yl)-6-(6-(trifluoromethyl)pyridin-3-yl)pyrido[3,4-d]pyrimidin-4(3H)-one;
15. 3-((1S,2R)-2-hydroxycyclohexyl)-8-(pyridin-3-yl)-6-(4-(trifluoromethyl)phenyl)pyrido[3,4-d]pyrimidin-4(3H)-one;
16. 3-((1R,2S)-2-hydroxycyclohexyl)-8-(pyridin-3-yl)-6-(4-(trifluoromethyl)phenyl)pyrido[3,4-d]pyrimidin-4(3H)-one;
17. 3-((1S,2R)-2-hydroxycyclohexyl)-8-(1-methyl-1H-pyrazol-4-yl)-6-(4-(trifluoromethyl)phenyl)pyrido[3,4-d]pyrimidin-4(3H)-one;
18. 3-((1R,2S)-2-hydroxycyclohexyl)-8-(1-methyl-1H-pyrazol-4-yl)-6-(4-(trifluoromethyl)phenyl)pyrido[3,4-d]pyrimidin-4(3H)-one;
19. 3-((1R,2S)-2-hydroxycyclohexyl)-8-(1-methyl-1H-pyrazol-4-yl)-6-(4-(trifluoromethyl)phenyl)pyrido[3,4-d]pyrimidin-4(3H)-one;
20. 6-(4-chlorophenyl)-3-((1S,2R)-2-hydroxycyclohexyl)-8-(1-methyl-1H-pyrazol-4-yl)pyrido[3,4-d]pyrimidin-4(3H)-one;
21. 6-(4-chlorophenyl)-3-((1S,2R)-2-hydroxycyclohexyl)-8-(pyridin-3-yl)pyrido[3,4-d]pyrimidin-4(3H)-one;
22. 8-(1-methyl-1H-pyrazol-4-yl)-3-(3,3,3-trifluoro-2-hydroxypropyl)-6-(4-(trifluoromethyl)phenyl)pyrido[3,4-d]pyrimidin-4(3H)-one;
23. 6-(4-chlorophenyl)-8-(1-methyl-1H-pyrazol-4-yl)-3-(3,3,3-trifluoro-2-hydroxypropyl)pyrido[3,4-d]pyrimidin-4(3H)-one;
24. 6-(4-chlorophenyl)-8-(pyridin-3-yl)-3-(3,3,3-trifluoro-2-hydroxypropyl)pyrido[3,4-d]pyrimidin-4(3H)-one;
25. 8-(pyridin-3-yl)-3-(3,3,3-trifluoro-2-hydroxypropyl)-6-(4-(trifluoromethyl)phenyl)pyrido[3,4-d]pyrimidin-4(3H)-one;
26. 6-(4-chlorophenyl)-3-(3-hydroxyphenyl)-8-(1-methyl-1H-pyrazol-4-yl)pyrido[3,4-d]pyrimidin-4(3H)-one;
27. 3-(3-hydroxyphenyl)-8-(1-methyl-1H-pyrazol-4-yl)-6-(4-(trifluoromethyl)phenyl)pyrido[3,4-d]pyrimidin-4(3H)-one;
28. 6-(4-chlorophenyl)-3-((1R,3S)-3-hydroxycyclopentyl)-8-(pyridin-3-yl)pyrido[3,4-d]pyrimidin-4(3H)-one;
29. 3-((1R,3S)-3-hydroxycyclopentyl)-8-(pyridin-3-yl)-6-(4-(trifluoromethyl)phenyl)pyrido[3,4-d]pyrimidin-4(3H)-one;
30. 6-(4-chlorophenyl)-3-((1R,3S)-3-hydroxycyclopentyl)-8-(1-methyl-1H-pyrazol-4-yl)pyrido[3,4-d]pyrimidin-4(3H)-one;
31. 3-((1R,3S)-3-hydroxycyclopentyl)-8-(1-methyl-1H-pyrazol-4-yl)-6-(4-(trifluoromethyl)phenyl)pyrido[3,4-d]pyrimidin-4(3H)-one;
32. 6-(4-chlorophenyl)-3-((1S,3R)-3-hydroxycyclopentyl)-8-(pyridin-3-yl)pyrido[3,4-d]pyrimidin-4(3H)-one;
33. 3-((1S,3R)-3-hydroxycyclopentyl)-8-(pyridin-3-yl)-6-(4-(trifluoromethyl)phenyl)pyrido[3,4-d]pyrimidin-4(3H)-one;
34. 6-(4-chlorophenyl)-3-((1S,3R)-3-hydroxycyclopentyl)-8-(1-methyl-1H-pyrazol-4-yl)pyrido[3,4-d]pyrimidin-4(3H)-one;
35. 3-((1S,3R)-3-hydroxycyclopentyl)-8-(1-methyl-1H-pyrazol-4-yl)-6-(4-(trifluoromethyl)phenyl)pyrido[3,4-d]pyrimidin-4(3H)-one;
36. 1-(6-(4-chlorophenyl)-4-oxo-8-(pyridin-3-yl)pyrido[3,4-d]pyrimidin-3(4H)-yl)-2-methylpropan-2-yl acetate;
37. 2-methyl-1-(4-oxo-8-(pyridin-3-yl)-6-(4-(trifluoromethyl)phenyl)pyrido[3,4-d]pyrimidin-3(4H)-yl)propan-2-yl acetate;
38. 6-(4-chlorophenyl)-3-(2-hydroxy-2-methylpropyl)-8-(pyridin-3-yl)pyrido[3,4-d]pyrimidin-4(3H)-one;
39. 3-(2-hydroxy-2-methylpropyl)-8-(pyridin-3-yl)-6-(4-(trifluoromethyl)phenyl)pyrido[3,4-d]pyrimidin-4(3H)-one;
40. 3-(2-hydroxy-2-methylpropyl)-8-(pyridin-3-yl)-6-(6-(trifluoromethyl)pyridin-3-yl)pyrido[3,4-d]pyrimidin-4(3H)-one;
41. 6-(4-chlorophenyl)-3-(1-hydroxy-3-methylbutan-2-yl)-8-(pyridin-3-yl)pyrido[3,4-d]pyrimidin-4(3H)-one;
42. 3-(1-hydroxy-3-methylbutan-2-yl)-8-(pyridin-3-yl)-6-(4-(trifluoromethyl)phenyl)pyrido[3,4-d]pyrimidin-4(3H)-one;
43. 3-(1-hydroxy-3-methylbutan-2-yl)-8-(pyridin-3-yl)-6-(6-(trifluoromethyl)pyridin-3-yl)pyrido[3,4-d]pyrimidin-4(3H)-one;
44. (S)-2-((6-(4-chlorophenyl)-2-(pyridin-3-yl)pyrimidin-4-yl)amino)propan-1-ol;
44. 3-(1-hydroxypropan-2-yl)-8-(pyridin-3-yl)-6-(4-(trifluoromethyl)phenyl)pyrido[3,4-d]pyrimidin-4(3H)-one;
45. 3-(1-hydroxypropan-2-yl)-8-(1-methyl-1H-pyrazol-4-yl)-6-(4-(trifluoromethyl)phenyl)pyrido[3,4-d]pyrimidin-4(3H)-one;
46. 6-(4-chlorophenyl)-3-(1-hydroxypropan-2-yl)-8-(pyridin-3-yl)pyrido[3,4-d]pyrimidin-4(3H)-one;
47. 2-(6-(4-chlorophenyl)-4-oxo-8-(pyridin-3-yl)pyrido[3,4-d]pyrimidin-3(4H)-yl)propyl diethyl phosphate;
48. 6-(4-chlorophenyl)-3-(1-hydroxypropan-2-yl)-8-(1-methyl-1H-pyrazol-4-yl)pyrido[3,4-d]pyrimidin-4(3H)-one;
49. 3-(1-hydroxypropan-2-yl)-8-(pyridin-3-yl)-6-(4-(trifluoromethoxy)phenyl)pyrido[3,4-d]pyrimidin-4(3H)-one;
50. 3-(1-hydroxypropan-2-yl)-8-(pyridin-3-yl)-6-(6-(trifluoromethyl)pyridin-3-yl)pyrido[3,4-d]pyrimidin-4(3H)-one;
51. 6-(4-chlorophenyl)-3-(1-hydroxybutan-2-yl)-8-(pyridin-3-yl)pyrido[3,4-d]pyrimidin-4(3H)-one;
52. 6-(4-chlorophenyl)-3-(1-hydroxybutan-2-yl)-8-(1-methyl-1H-pyrazol-4-yl)pyrido[3,4-d]pyrimidin-4(3H)-one;
53. 3-(1-hydroxybutan-2-yl)-8-(pyridin-3-yl)-6-(4-(trifluoromethyl)phenyl)pyrido[3,4-d]pyrimidin-4(3H)-one;
54. 3-(1-hydroxybutan-2-yl)-8-(1-methyl-1H-pyrazol-4-yl)-6-(4-(trifluoromethyl)phenyl)pyrido[3,4-d]pyrimidin-4(3H)-one;
55. 6-(4-chlorophenyl)-8-(3-fluorophenyl)-3-(1-hydroxybutan-2-yl)pyrido[3,4-d]pyrimidin-4(3H)-one;
56. 6-(4-chlorophenyl)-3-((1r,4r)-4-hydroxycyclohexyl)-8-(pyridin-3-yl)pyrido[3,4-d]pyrimidin-4(3H)-one;
57. 3-((1r,4r)-4-hydroxycyclohexyl)-8-(pyridin-3-yl)-6-(4-(trifluoromethyl)phenyl)pyrido[3,4-d]pyrimidin-4(3H)-one;
58. 6-(4-chlorophenyl)-3-((1s,4s)-4-hydroxycyclohexyl)-8-(pyridin-3-yl)pyrido[3,4-d]pyrimidin-4(3H)-one;
59. 3-(1-hydroxypropan-2-yl)-8-(1-methyl-1H-pyrazol-4-yl)-6-(4-(trifluoromethyl)phenyl)-2,3-dihydropyrido[3,4-d]pyrimidin-4(1H)-one;
60. 6-(4-chlorophenyl)-3-(2,3-dihydroxypropyl)-8-(pyridin-3-yl)pyrido[3,4-d]pyrimidin-4(3H)-one;
61. 6-(4-chlorophenyl)-3-(3-hydroxyphenyl)-8-(pyridin-3-yl)pyrido[3,4-d]pyrimidin-4(3H)-one;
62. 3-(3-hydroxyphenyl)-8-(pyridin-3-yl)-6-(4-(trifluoromethyl)phenyl)pyrido[3,4-d]pyrimidin-4(3H)-one;
63. 6-(4-chlorophenyl)-3-(3-hydroxycyclohexyl)-8-(pyridin-3-yl)pyrido[3,4-d]pyrimidin-4(3H)-one;
64. 6-(4-chlorophenyl)-3-(3-hydroxycyclohexyl)-8-(1-methyl-1H-pyrazol-4-yl)pyrido[3,4-d]pyrimidin-4(3H)-one;
65. 3-((1R,3S)-3-hydroxycyclopentyl)-8-(pyridin-3-yl)-6-(6-(trifluoromethyl)pyridin-3-yl)pyrido[3,4-d]pyrimidin-4(3H)-one;
65. 3-((1R,3S)-3-hydroxycyclopentyl)-8-(pyridin-3-yl)-6-(6-(trifluoromethyl)pyridin-3-yl)pyrido[3,4-d]pyrimidin-4(3H)-one;
66. 3-(2,3-dihydroxypropyl)-8-(pyridin-3-yl)-6-(4-(trifluoromethyl)phenyl)pyrido[3,4-d]pyrimidin-4(3H)-one;
67. 6-(4-chlorophenyl)-3-(2,3-dihydroxypropyl)-8-(pyridin-3-yl)pyrido[3,4-d]pyrimidin-4(3H)-one;
68. 3-(2,3-dihydroxypropyl)-6-(4-(4-methylpiperazin-1-yl)phenyl)-8-(pyridin-3-yl)pyrido[3,4-d]pyrimidin-4(3H)-one
69. 3-(1,3-dihydroxypropan-2-yl)-8-(pyridin-3-yl)-6-(4-(trifluoromethyl)phenyl)pyrido[3,4-d]pyrimidin-4(3H)-one;
70. 6-(4-chlorophenyl)-3-(1,3-dihydroxypropan-2-yl)-8-(pyridin-3-yl)pyrido[3,4-d]pyrimidin-4(3H)-one;
71. 6-(6-chloropyridin-3-yl)-3-((1R,3S)-3-hydroxycyclopentyl)-8-(pyridin-3-yl)pyrido[3,4-d]pyrimidin-4(3H)-one
72. 3-((1R,3S)-3-hydroxycyclopentyl)-8-(pyridin-3-yl)-6-(2-(trifluoromethyl)pyrimidin-5-yl)pyrido[3,4-d]pyrimidin-4(3H)-one, TFA salt;
73. 3-((1R,3S)-3-hydroxycyclopentyl)-6-(4-morpholinophenyl)-8-(pyridin-3-yl)pyrido[3,4-d]pyrimidin-4(3H)-one;
74. 6-(4′-chloro-[1,1′-biphenyl]-4-yl)-3-(1-hydroxypropan-2-yl)-8-(pyridin-3-yl)pyrido[3,4-d]pyrimidin-4(3H)-one;
75. 3-(1-hydroxypropan-2-yl)-6-(4-morpholinophenyl)-8-(pyridin-3-yl)pyrido[3,4-d]pyrimidin-4(3H)-one;
76. 3-(2-(methylsulfonyl)ethyl)-8-(pyridin-3-yl)-6-(4-(trifluoromethyl)phenyl)pyrido[3,4-d]pyrimidin-4(3H)-one;
76. 3-(2-(methylsulfonyl)ethyl)-8-(pyridin-3-yl)-6-(4-(trifluoromethyl)phenyl)pyrido[3,4-d]pyrimidin-4(3H)-one;
77. 6-(4-chlorophenyl)-3-(2-(methylsulfonyl)ethyl)-8-(pyridin-3-yl)pyrido[3,4-d]pyrimidin-4(3H)-one;
78. 3-(1,1-dioxidotetrahydro-2H-thiopyran-4-yl)-6-(4-morpholinophenyl)-8-(pyridin-3-yl)pyrido[3,4-d]pyrimidin-4(3H)-one;
79. 3-(2-(methylsulfonyl)ethyl)-6-(4-morpholinophenyl)-8-(pyridin-3-yl)pyrido[3,4-d]pyrimidin-4(3H)-one;
80. 3-(1,3-dihydroxypropan-2-yl)-6-(4-morpholinophenyl)-8-(pyridin-3-yl)pyrido[3,4-d]pyrimidin-4(3H)-one;
80. 3-(1,3-dihydroxypropan-2-yl)-6-(4-morpholinophenyl)-8-(pyridin-3-yl)pyrido[3,4-d]pyrimidin-4(3H)-one;
81. (R)-3-(2,3-dihydroxypropyl)-8-(pyridin-3-yl)-6-(4-(trifluoromethyl)phenyl)pyrido[3,4-d]pyrimidin-4(3H)-one;
82. 3-(2,3-dihydroxypropyl)-6-(4-morpholinophenyl)-8-(pyridin-3-yl)pyrido[3,4-d]pyrimidin-4(3H)-one;
83. 2-(6-(4-chlorophenyl)-4-oxo-8-(pyridin-3-yl)pyrido[3,4-d]pyrimidin-3(4H)-yl)propanoic acid, 2,2,2-trifluoroacetic acid salt;
84. 2-(4-oxo-8-(pyridin-3-yl)-6-(4-(trifluoromethyl)phenyl)pyrido[3,4-d]pyrimidin-3(4H)-yl)propanoic acid, 2,2,2-trifluoroacetic acid salt;
86. N-(2-(4-oxo-8-(pyridin-3-yl)-6-(4-(trifluoromethyl)phenyl)pyrido[3,4-d]pyrimidin-3(4H)-yl)propyl)acetamide;
85. 3-(1-aminopropan-2-yl)-8-(pyridin-3-yl)-6-(4-(trifluoromethyl)phenyl)pyrido[3,4-d]pyrimidin-4(3H)-one;
86. N-(2-(4-oxo-8-(pyridin-3-yl)-6-(4-(trifluoromethyl)phenyl)pyrido[3,4-d]pyrimidin-3(4H)-yl)propyl)acetamide;
87. 3-(1-aminopropan-2-yl)-6-(4-chlorophenyl)-8-(pyridin-3-yl)pyrido[3,4-d]pyrimidin-4(3H)-one;
88. N-(2-(6-(4-chlorophenyl)-4-oxo-8-(pyridin-3-yl)pyrido[3,4-d]pyrimidin-3(4H)-yl)propyl)acetamide;
89. N-(2-(6-(4-chlorophenyl)-4-oxo-8-(pyridin-3-yl)pyrido[3,4-d]pyrimidin-3(4H)-yl)propyl)methanesulfonamide;
90. 3-(1-aminopropan-2-yl)-6-(4-morpholinophenyl)-8-(pyridin-3-yl)pyrido[3,4-d]pyrimidin-4(3H)-one;
91. N-(2-(6-(4-morpholinophenyl)-4-oxo-8-(pyridin-3-yl)pyrido[3,4-d]pyrimidin-3(4H)-yl)propyl)methanesulfonamide;
92. N-(2-(6-(4-morpholinophenyl)-4-oxo-8-(pyridin-3-yl)pyrido[3,4-d]pyrimidin-3(4H)-yl)propyl)acetamide;
93. 3-(piperidin-4-yl)-8-(pyridin-3-yl)-6-(4-(trifluoromethyl)phenyl)pyrido[3,4-d]pyrimidin-4(3H)-one;
94. 6-(4-chlorophenyl)-3-(1-(methylsulfonyl)piperidin-4-yl)-8-(pyridin-3-yl)pyrido[3,4-d]pyrimidin-4(3H)-one;
95. 6-(4-chlorophenyl)-3-(1-(cyclopropylsulfonyl)piperidin-4-yl)-8-(pyridin-3-yl)pyrido[3,4-d]pyrimidin-4(3H)-one; and
96. 3-(1-acetylpiperidin-4-yl)-6-(4-chlorophenyl)-8-(pyridin-3-yl)pyrido[3,4-d]pyrimidin-4(3H)-one.

9. A pharmaceutical composition comprising the compound of formula (I) according to claim 1, or an enantiomer, diastereomer, racemate, solvate, hydrate, or pharmaceutically acceptable salt thereof and a pharmaceutically acceptable carrier.

10-15. (canceled)

16. A method of modulating AhR activity in a subject comprising administering activity a therapeutically effective amount of the compound of formula (I) according to claim 1 or an enantiomer, diastereomer, racemate, solvate, hydrate, or pharmaceutically acceptable salt thereof.

17. A method of preventing or treating a disease or condition mediated by aryl hydrocarbon receptor (AhR) in a subject comprising administering a therapeutically effective amount of the compound of formula (I) according to claim 1 or an enantiomer, diastereomer, racemate, solvate, hydrate, or pharmaceutically acceptable salt thereof.

18. The method according to claim 17, wherein the disease or condition mediated by aryl hydrocarbon receptor (AhR) is cancer, cancerous conditions, tumor, fibrotic disorders, or conditions with dysregulated immune responses or other disorders associated with aberrant AhR signaling.

19. The method according to claim 18, wherein the cancer is selected from a group consisting of a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, and a chronic myeloblastic leukemia.

20. The method according to claim 18, wherein the fibrotic disorder is selected from a group consisting of hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis, scleroderma, morphea, keloids, hypertrophic scarring, naevi, diabetic retinopathy, proliferative vitroretinopathy and sarcoidosis.

21. The method according to claim 18, wherein the condition with dysregulated immune responses is selected from a group consisting of sepsis, multiple organ failure, inflammatory disorders of the kidney, chronic intestinal inflammations, pancreatitis, peritonitis, inflammatory skin disorders and inflammatory eye disorders, rheumatoid diseases, systemic lupus erythematosus and multiple sclerosis.

22. A method of inhibiting proliferation, tissue invasion, metastasis and angiogenesis of cancer cells in a subject having a cancer, a cancerous condition, or a tumor, comprising administering a therapeutically effective amount of the compound of formula (I) according to claim 1 or an enantiomer, diastereomer, racemate, solvate, hydrate, or pharmaceutically acceptable salt thereof.
